(19) United States
(12) Patent Application Publication
(10) Pub. No.: US 2023/0218710 A1
(43) Pub. Date: Jul. 13, 2023

(54) METHOD OF TREATING CORONAVIRUS INFECTIONS

(71) Applicant: COHBAR, INC., Menlo Park, CA (US)

(72) Inventor: Kenneth Cundy, Atherton, CA (US)

(21) Appl. No.: 17/915,429

(22) PCT Filed: Apr. 15, 2021
(86) PCT No.: PCT/US2021/027390

§ 371 Date: Sep. 28, 2022

Related U.S. Application Data
(60) Provisional application No. 63/150,415, filed on Feb. 17, 2021.
(60) Provisional application No. 63/122,397, filed on Dec. 7, 2020.
(60) Provisional application No. 63/064,333, filed on Aug. 11, 2020.
(60) Provisional application No. 63/035,537, filed on Jun. 5, 2020.
(60) Provisional application No. 63/010,627, filed on Apr. 15, 2020.

(51) Int. Cl. A61K 38/10; A61K 38/08; A61P 11/00; A61P 29/00

(52) U.S. Cl. CPC A61K 38/10; A61K 38/08; A61P 11/00; A61P 29/00

(57) ABSTRACT

The disclosures herein relate a method of treating coronavirus infections. More specifically disclosed herein are peptides effective as apelin receptor agonists. Also disclosed herein are peptides effective in the treatment of acute respiratory distress syndrome (ARDS) induced by COVID-19.

### PRIORITY

This application claims priority benefit of United States provisional patent application Nos. 63/150,415, filed on Feb. 17, 2021; 63/122,397, filed on Dec. 7, 2020; 63/064,333, filed on Aug. 11, 2020; 63/035,537, filed on Jun. 5, 2020; and 63/010,627, filed on Apr. 15, 2020. Each of those applications is hereby incorporated by reference in its entirety.

### INCORPORATION BY REFERENCE OF MATERIAL SUBMITTED ELECTRONICALLY

This application incorporates by reference in its entirety a computer-readable nucleotide/amino acid sequence listing identified as one 29,120 byte ASCII (text) file named “CBA075A_Seqlisting.txt,” created on Apr. 13, 2021. To the extent differences exist between information/description of sequences in the specification and information in the Sequence Listing, the specification is controlling.

### TECHNICAL FIELD

This disclosure relates to a method of treating infectious diseases such as coronavirus infections. More specifically disclosed are peptides effective as apelin receptor agonists. Also disclosed are peptides effective for the treatment of acute respiratory distress syndrome (ARDS) including ARDS induced by COVID-19. Also disclosed are peptides effective for the treatment of acute lung injury and fluid accumulation in the lungs.

### BACKGROUND

The coronavirus designated SARS-CoV-2 spread from China in late 2019, inducing a global pandemic of severe acute respiratory disease, referred to as COVID-19 (Zhou M et al. Coronavirus disease 2019 (COVID-19): a clinical update Front Med. 2020 Apr. 2: 1-10). The most severe cases of COVID-19 are characterized by intense and dysregulated inflammation, pneumonia, lung damage, and severe respiratory distress, requiring high-pressure oxygen therapy and eventually prolonged ventilation. The prognosis for elderly patients with severe or critical disease is poor, with a reported mortality rate close to 20% (Wang L et al. Coronavirus Disease 2019 in elderly patients: characteristics and prognostic factors based on 4-week follow-up. J Infect., 2020 June; 80(6): 639-645, [Epub ahead of print 2020 Mar. 30 doi: 10.1016/j.jinf.2020.03.019]). Surviving patients can also be expected to have lasting damage as a result of their severe inflammatory response. Damage induced in lung tissue and other organs by SARS-CoV-2 infection appears driven by a cytokine response syndrome or “cytokine storm” involving intense release of high levels of pro-inflammatory cytokines such as interleukin-6 (IL-6) from cells of the host (McGonagle D et al. Interleukin-6 use in COVID-19 pneumonia related macrophage activation syndrome. Autoimmun Rev. 2020 June; 19(6): 102537 (published online 2020 Apr. 3:102537. doi: 10.1016/j.autrev.2020.102537). Additionally, local inflammatory damage to lung tissue and microvasculature leads to pulmonary vascular leakage and consequent accumulation of fluid in the lungs, reducing oxygen intake and inducing hypoxia, which in turn exacerbates the inflammatory response (Wu G et al. Hypoxia Exacerbates Inflammatory Acute Lung Injury via the Toll-Like Receptor 4 Signaling Pathway. Front Immunol. 2018; 9: 1667). The excessive pro-inflammatory cytokine release induced by SARS-CoV-2 infection can lead to multi-organ failure, a significant cause of death for infected patients with severe disease.

In 2003, the SARS-CoV was identified as a new emerging infectious pathogen responsible for severe acute respiratory syndrome in humans. During winter 2002/2003, more than 8000 people were infected by this highly infectious agent and 10% of them died. The SARS-CoV mainly targets the epithelial cells, the respiratory tract being the primary site of infection. One of the major pathological features of SARS-CoV infection is diffuse alveolar damage (DAD) of the human lung, more prominent in the terminal stage, with sometimes, abrupt deterioration of the lung epithelium. The SARS pathogen triggered atypical pneumonia characterized by high fever, severe dyspnea and the development of acute severe lung failure. Moreover, influenza such as the Spanish flu and the emergence of new respiratory disease viruses have caused high lethality among infected individuals due to acute lung failure. There is a need for more effective therapies modulating viral related lung disease.

Apelin is a peptide hormone involved in regulation of fluid homeostasis and cardiovascular function that has additional anti-oxidative and anti-inflammatory activities. However, the very short in vivo half-life of the natural apelin peptide limits its potential clinical applications. In animal studies, the active form of apelin, apelin-13, has been shown to reduce pulmonary vascular leakage resulting from LPS-induced acute lung tissue damage in animals (Petrescu B C et al. Apelin effects on lipopolysaccharide-increased pulmonary permeability in rats. Rev Med Chir Soc Med Nat Iasi. 2010 January-March; 114(1):163-9). Apelin-13 also attenuated tissue damage in the liver of rats induced by LPS, leading to reduction in apoptosis, ROS production, hepatic macrophage infiltration, and expression of TNFα and IL-6 (Zhou H et al. Fc-apelin fusion protein attenuates lipopolysaccharide-induced liver injury in mice. Sci Rep. 2018 Jul. 30; 8(1):11428). There is a need for more effective therapies modulating apelin-mediated diseases and diseases affected by apelin signaling, such as ARDS and acute lung disease.

The inventors have identified therapeutically useful isolated peptides with unexpected properties based on mitochondrial DNA and conceived novel analogs and derivatives with improved properties. Therefore, the present invention provides the use of apelin receptor agonists for the preparation of a medicament for the treatment of severe acute lung injury, of lung oedemas, and lung injuries and failures connected with infection with coronavirus.

### SUMMARY

Disclosed are materials and methods useful for treating patients or subjects with coronavirus infections and other infections that cause or contribute to acute lung injury. The present disclosure moreover includes peptides effective as apelin receptor agonists. Also disclosed are peptides effective for the treatment of subjects with acute respiratory distress induced by COVID-19. Also disclosed is a treatment that can reduce the accumulation of fluid in lungs and decrease the release of pro-inflammatory cytokines in response to a viral infection.

The present disclosure includes materials and methods for treating an apelin-mediated disease or medical condition (e.g., infectious diseases and lung injury) in a patient using peptides and compositions described herein. Also disclosed are peptides comprising amino acid sequences of Formulas I-IV and II′-III′ that exhibit activity in agonizing the apelin receptor. Also disclosed are apelin agonist peptides comprising amino acid sequences SEQ ID NO: 1-64 and 69-79, analogs and derivatives thereof.

The present disclosure moreover includes compositions, including pharmaceutical compositions, comprising amino acid sequences of Formulas I-IV and II′-III′, and/or SEQ ID NOs: 1-64 and 69-79, analogs and derivatives thereof described herein and a pharmaceutically acceptable excipient.

The present disclosure includes materials and methods for modulating activation of RAS or phosphorylation of MEK1 or ERK1/2 using peptides and compositions described herein.

To the extent that embodiments, details, or variations are described herein with reference to one genus of peptides (e.g., Formula I peptides), it should be understand that the same embodiments, details, and variations are intended to apply to other genera, unless the application or context explicitly indicates otherwise.

Various details and aspects are described herein as treating or methods of treating. In all such circumstances, it should be understood that related or equivalent aspects include the peptides, analogs, derivatives, or compositions described herein for use in treatment; and the peptides, analogs, derivatives, or compositions described herein for use in the manufacture of medicaments for treatment of diseases or conditions described herein.

The headings herein are for the convenience of the reader and not intended to be limiting. Other aspects of the invention will be apparent from the detailed description and claims that follow.

### DETAILED DESCRIPTION

Disclosed is a method of treating coronavirus infections. Also disclosed are peptides effective for the treatment of acute respiratory distress induced by COVID-19.

There is considerable evidence that respiratory viral infections can prime host cells to react to a subsequent bacterial infection by producing a similar cytokine release syndrome to that observed with COVID-19 infection. The prior severe acute respiratory syndrome (SARS) epidemic was caused by a novel human coronavirus (CoV), designated SARS-CoV, which was a highly contagious respiratory disease with the lungs as the major target. SARS-CoV infection was also characterized by an intense, dysregulated local inflammatory response leading to devastating lung pathology (Tseng C T et al. Severe acute respiratory syndrome and the innate immune responses: modulation of effector cell function without productive infection. J Immunol. 2005 Jun. 15; 174(12):7977-85). In human macrophages and dendritic cells, exposure to SARS-CoV primed the cells to respond to a suboptimal dose of bacterial LPS, resulting in massive release of IL-6 and IL-12. It has also been shown that infection of pigs with porcine respiratory coronavirus (PRCV) effectively primed the host immune system to respond to low doses of LPS with a cytokine storm (Van Reeth K et al. In vivo studies on cytokine involvement during acute viral respiratory disease of swine: troublesome but rewarding. Vet Immunol Immunopathol. 2002 Sep. 10; 87(3-4):161-8). Multifactorial respiratory disease was reproduced by inoculations with a subclinical dose of PRCV followed by LPS from Escherichia coli. The subclinical dose of virus alone did not induce the cytokine response, but when followed by bacterial LPS there was a clear potentiation of disease and excessive production of TNF-alpha, IL-1 and IL-6.

Disclosed is a treatment that can reduce the accumulation of fluid in lungs and decrease the release of pro-inflammatory cytokines in response to a viral infection. In one aspect, a treatment is disclosed that can reduce the incidence of a cytokine storm in a subject with a coronavirus infection, whether cytokines were induced by the virus itself or as a consequence of priming of cells and subsequent bacterial infection.

Disclosed is a treatment of bacteria-induced acute lung damage. Administration of bacterial lipopolysaccharide (LPS) to animals by intratracheal or nasogastric dosing induces an acute respiratory distress syndrome that is similar in nature to the effects produced by coronavirus infections, including SARS-CoV-2 infection. LPS-induced acute lung damage includes pulmonary vascular leakage, cellular apoptosis, ROS production, macrophage infiltration, and excessive secretion of pro-inflammatory cytokines such as TNFα and IL-6 (Tseng C T et al. Severe acute respiratory syndrome and the innate immune responses: modulation of effector cell function without productive infection. J Immunol. 2005 Jun. 15; 174(12):7977-85). In one aspect, a method of modulating pro-inflammatory cytokine secretion is described.

In one aspect, a method of treating acute respiratory syndrome caused by a virus is described. Severe acute respiratory syndrome (SARS) is a highly contagious disease caused by SARS-associated coronavirus (SARS-CoV). The acute respiratory syndrome may be caused by a SARS-associated coronavirus (SARS-CoV). In one embodiment, the acute respiratory syndrome may be caused by a coronaviridae virus. The acute respiratory syndrome may be caused by a MERS-CoV, HCoV-229E and/or NL63. The virus that causes respiratory disease may also be SARS-CoV-2 infection or COVID-19. Coronaviruses (Coy) historically are known to cause relatively mild upper respiratory tract infections, and account for approximately 30% of the cases of the common cold in humans. However, in CoV, severe acute respiratory syndrome coronavirus (SARS-CoV) causes severe respiratory distress in humans. In humans, SARS-CoV peak viral load is reached by about 10 days post-infection, thus offering an opportunity for effective post-exposure treatment.

The present disclosure moreover includes peptides effective as apelin mimetics or apelin receptor agonists. Apelin is a peptide hormone involved in regulation of fluid homeostasis and cardiovascular function that has additional anti-oxidative and anti-inflammatory activities. However, the very short in vivo half-life of the natural apelin peptide limits its potential clinical applications. In animal studies, the active form of apelin, apelin-13, has been shown to reduce pulmonary vascular leakage resulting from LPS-induced acute lung tissue damage (Petrescu B C et al. Apelin effects on lipopolysaccharide-increased pulmonary permeability in rats. Rev Med Chir Soc Med Nat Iasi. 2010 January-March; 114(1):163-9). Apelin-13 also attenuated tissue damage in the liver of rats induced by LPS, leading to reduction in apoptosis, ROS production, hepatic macrophage infiltration, and expression of TNFα and IL-6 (Zhou H et al. Fc-apelin fusion protein attenuates lipopolysaccharide-induced liver injury in mice. Sci Rep. 2018 Jul. 30; 8 (1):11428).

The improved apelin analogs of the present invention are agonists of the apelin receptor. In one embodiment, the improved apelin analogs are capable of inducing a receptor response of the same magnitude as the natural peptide apelin-13. The improved apelin analogs of the present invention can be used to treat patients with bacterial and viral infections, including acute respiratory viral infections such coronavirus infections (e.g., infections associated with SARS-CoV, SARS-CoV-2 and potentially future coronaviruses that induce similar effects), by reducing disease-related effects such as fluid accumulation in the lungs, recruitment of macrophages to lung tissue, apoptosis of lung cells, generation of ROS, and secretion of pro-inflammatory cytokines, including but not limited to IL-6 and TNFα. Other pro-inflammatory cytokines are IL-1β, IL-2, IL-4, IL-5, IL-17α, IL17γ, IL-23, IFNg, MCP-1, MIP-1α, MIP-3α, and IL-8. The improved apelin analogs of the present invention may be used to treat infections that damage lungs and other organs, including bacterial infections, viral infections, or infections involving both viruses and bacteria. The improved apelin analogs of the present invention have greater metabolic stability than the natural apelin-13 peptide and provide a more extended protection from the damaging effects of an infection such as with SARS-CoV-2 than can be achieved by administration of the natural apelin peptide.

In one embodiment, a method of treating a patient or subject infected with, or suspected of having an infection with, coronavirus with apelin analogs is described. In one aspect, a method of treating COVID-19 (also known as severe acute respiratory syndrome coronavirus 2 or SARS-CoV-2 infection) with apelin analogs is described.

Both human apelin receptor (APJ) and apelin have been implicated as the key mediators of physiological responses to multiple homeostatic perturbations, including cardiovascular control, water balance, hypothalamic-pituitary-adrenal (HP A) axis regulation and metabolic homeostasis. Elevated levels of apelin have been detected in many pathological states or disease processes, such as heart disease, atherosclerosis, tumor angiogenesis and diabetes. However, in many systems, apelin has been shown to have positive effects, for example in the cardiovascular system, where it has a cardioprotective effect. It has also been associated with sepsis related injury, cerebral ischemic events, thrombin related aggregation and UVB radiation recovery. See Tian et al., Frontiers in Neurology, 11:75 (2020); Sawane et al, AJP, 179(6), 2691-2697 (2011); Luo, et al., Int. J of Molecular Med., 42, 1161-1167 (2018); and Adam et al. Blood, 127, (7) 908-920, February 2016.

The apelinergic system has been implicated in tumor neoangiogenesis. Apelin agonists may have therapeutic effects in ischemia recovery due to vessel regeneration and endothelial proliferation and blood vessel diameter regulation.

APJ is widely distributed and present at high levels in lung, heart, adrenal cortex, renal medulla, ovary and uterus of animals (Pope G R, et al. Central and peripheral apelin receptor distribution in the mouse: Species differences with rat. Peptides. 2012 January; 33(1): 139-148). APJ is also localized in the hypothalamic pPVN and the anterior pituitary gland, key areas involved in the stress response. The presence of APJ and apelin in VP- and CRH-containing hypothalamic nuclei, which are pivotal to the HPA axis responses to stress, suggests a role for apelin/APJ in neuroadenohypophysial hormone release.

Apelin and APJ are regulators of central and peripheral responses to multiple homeostatic perturbations such as cardiovascular control and function; angiogenesis; fluid homeostasis; water balance; hypothalamic-pituitary-adrenal (HPA) axis regulation; metabolic homeostasis; energy metabolism; and kidney function. APJ-apelin signaling plays a role in the maintenance of pulmonary vascular homeostasis (see, e.g., Kim supra). Evidence also points to a nexus between apelinergic system (e.g., apelin and APJ receptor) and the treatment of conditions such as sepsis, septic shock, and renal failure (see, e.g., Coquerel, D., et al., Critical Care 2018, 22: 10). As another example, apelin, synthesized and secreted by adipocytes, has been described as a beneficial adipokine. Therefore, the peptides of Formula I-IV and II′-III′ are effective as treatment of pulmonary hypertension (e.g., PAH); heart failure; type II diabetes; renal failure; sepsis; and systemic hypertension.

The present invention is based on the discovery of a series of potent agonists of the apelin receptor (APJ). In further aspects, the peptides of the current invention are used for the treatment of apelin mediated diseases or disorders. In further aspects, the peptides of the current invention are used for the treatment of diseases including heart failure, chronic kidney disease, hypertension, and metabolic disorders.

One aspect of the invention is a method of preventing or treating in a subject an apelin-mediated disease or disorder, comprising administering to the subject a pharmaceutical a compound listed above, thereby preventing or treating the disease or disorder is also provided herein.

In further aspects the disease or disorder is CNS-dependent or CNS-independent disturbed fluid homeostasis, acute or chronic renal failure, hypertension, pulmonary hypertension, portal hypertension or systolic hypertension.

In other aspects, the disease or disorder is a vascular disease or disorder, vascular permeability, nonfunctional blood vessels, vascular hypertrophy, vascular remodeling, vascular stiffness, atherosclerosis, peripheral arterial occlusive disease (PAOD), restenosis, thrombosis, vascular permeability disorders, ischemia, reperfusion damage, ischemia or reperfusion damage of the heart, kidney or retina, or a combination thereof.

In certain aspects, the disease or disorder is thrombosis or thrombin-mediated platelet aggregation. The present apelin agonists can be used to maintain hemostasis and regulation of platelet function. The agonists can inhibit thrombin-mediated and collagen-mediated platelet activation. The peptides of the invention are anti-aggregation agents and anti-thrombotic agents. The peptides of the invention are useful for the prevention of platelet aggregation and thrombin mediated events.

In still other aspects, the disease or disorder is an infectious disease.

In certain aspects, the disease or disorder is a cardiovascular disease or disorder, coronary heart disease, stroke, heart failure, systolic heart failure, diastolic heart failure, diabetic heart failure, heart failure with preserved ejection fraction, cardiomyopathy, myocardial infarction, left ventricular dysfunction, left ventricular dysfunction after myocardial infarction, cardiac hypertrophy, myocardial remodeling, myocardial remodeling after infarction, myocardial remodeling after cardiac surgery or valvular heart disease.

In other aspects the disease or disorder is a metabolic disease or disorder, metabolic syndrome, insulin resistance, diabetes mellitus, diabetic late complications, diabetic macro- and micro-vasculopathies, diabetic nephropathy, diabetic retinopathy, diabetic neuropathies or cardiac autonomic neuropathy.

In further aspects, the invention includes a method of treating and/or preventing a disease or disorder selected from hypertension, endothelial dysfunction, damages to cardiovascular tissues, heart failure, coronary heart disease, ischemic and/or hemorrhagic stroke, macrovascular disease, microvascular disease, diabetic heart (including diabetic cardiomyopathy and heart failure as a diabetic complication) coronary heart disease, peripheral artery disease, peripheral arterial occlusive disease, pre-eclampsia, resistant hypertension, refractory hypertension, hypertensive crisis, blood or fetal-placental circulation, edematous diseases, pulmonary dysfunction, acute lung injury (ALI), acute respiratory distress syndrome (ARDS), trauma and/or burns, and/or ventilator induced lung injury (VI LI), pulmonary fibrosis, mountain sickness, chronic kidney diseases, acute kidney injury, lymphedema, lymphatic vessel regeneration, inflammatory bowel disease, inflammatory disease, or ocular disorders associated with disturbed vascular function, topical wounds, migraine, angiogenesis, degeneration of cartilage, osteoarthritis, and cancers.

In further aspects the APJ agonists reduce extravascular lung fluid accumulation, capillary-alveolar leakage, and hypoxemia. In further aspects the APJ agonists act as key regulators of central and peripheral responses to multiple homeostatic perturbations. In further aspects the APJ agonists regulate angiogenesis, fluid homeostasis or energy metabolism. In further aspects, the APJ agonists act as neuroendocrine modulators of the FIFA axis responses to stress. In further aspects the APJ agonists benefit cardiovascular function. In further aspects the peptides of the current invention are used for the treatment of acute lung injury.

The term “apelin mediated disease or disorder” as used herein includes any disease or disorder that is mediated by apelin. Examples of apelin mediated diseases or disorders include, but are not limited to, a cardiovascular disease or disorder, coronary heart disease, stroke, heart failure, systolic heart failure, diastolic heart failure, diabetic heart failure, heart failure with preserved ejection fraction, cardiomyopathy, myocardial infarction, left ventricular dysfunction, left ventricular dysfunction after myocardial infarction, cardiac hypertrophy, myocardial remodeling, myocardial remodeling after infarction, myocardial remodeling after cardiac surgery, valvular heart disease; a metabolic disease or disorder, metabolic syndrome, insulin resistance, diabetes mellitus, diabetic late complications, diabetic macro- and micro-vasculopathies, diabetic nephropathy, diabetic retinopathy, diabetic neuropathies, cardiac autonomic neuropathy; a disease or disorder is caused by CNS-dependent or CNS-independent disturbed fluid homeostasis, acute or chronic renal failure, hypertension, pulmonary hypertension, portal hypertension, systolic hypertension; a vascular disease or disorder, vascular permeability, nonfunctional blood vessels, vascular hypertrophy, vascular remodeling, vascular stiffness, atherosclerosis, peripheral arterial occlusive disease (PAOD), restenosis, thrombosis, vascular permeability disorders, ischemia, reperfusion damage, ischemia, reperfusion damage of the heart, kidney or retina, or a combination thereof.

One aspect of the invention is the use of the invention in treating a coronavirus infection in a subject, comprising administering to the subject a peptide, peptide analog, composition, nucleic acid, vector, expression vector, or host cell of the invention, in an amount effective to treat the coronavirus. One aspect is where the coronavirus is SARS or COVID-19. One aspect is where the treatment reduces the coronavirus-related acute lung injury.

In one aspect, a treatment is disclosed that can reduce the incidence of a cytokine storm in a subject with a pathogenic infection, whether cytokines were induced by the pathogen itself or as a consequence of priming of cells and subsequent bacterial infection.

The peptides of the invention are useful for treatment and/or prophylaxis of bacterial infection in humans or other animals by administering to the subject in need of a therapeutically effective amount of peptide of any of Formulas I-IV and II′-III′, or a pharmaceutically acceptable salt, or thereof. The peptides and methods of the invention are particularly well suited for human patients infected by pathogens that include Staphylococcus aureus, Escherichia coli, Klebsiella pneumoniae, Acinetobacter baumannii and Pseudomonas aeruginosa.

Examples of bacterial infections may include, but not limited to, upper respiratory infections, lower respiratory infections, ear infections, pleuropulmonary and bronchial infections, complicated urinary tract infections, uncomplicated urinary tract infections, intra-abdominal infections, cardiovascular infections, a blood stream infection, sepsis, bacteremia, CNS infections, skin and soft tissue infections, GI infections, bone and joint infections, genital infections, eye infections, or granulomatous infections. Examples of specific bacterial infections include, but not limited to, uncomplicated skin and skin structure infections (uSSSI), complicated skin and skin structure infections (cSSSI), catheter infections, pharyngitis, sinusitis, otitis externa, otitis media, bronchitis, empyema, pneumonia, community-acquired bacterial pneumoniae (CABP), hospital-acquired pneumonia (HAP), hospital-acquired bacterial pneumonia, ventilator-associated pneumonia (VAP), diabetic foot infections, vancomycin resistant enterococci infections, cystitis and pyelonephritis, renal calculi, prostatitis, peritonitis, complicated intra-abdominal infections (cIAI) and other inter-abdominal infections, dialysis-associated peritonitis, visceral abscesses, endocarditis, myocarditis, pericarditis, transfusion-associated sepsis, meningitis, encephalitis, brain abscess, osteomyelitis, arthritis, genital ulcers, urethritis, vaginitis, cervicitis, gingivitis, conjunctivitis, keratitis, endophthalmitis, an infection in cystic fibrosis patients or an infection of febrile neutropenic patients.

In one aspect disclosed herein is a method of treating, preventing, inhibiting, reducing the incidence of, ameliorating, or alleviating sepsis, or any combination thereof, in a subject in need, comprising the step of administering a composition comprising an early apoptotic cell population to said subject, wherein said administering treats, prevents, inhibits, reduces the incidence of, ameliorates, or alleviates sepsis in said subject.

In a related aspect, the sepsis comprises mild or severe sepsis. In some embodiments, the source of sepsis comprises pneumonia, an endovascular methicillin-resistant Staphylococcus aureus (MRS A) infection, sepsis-induced cardiomyopathy or a urinary tract infection (UTI).

In another related aspect, the method results in increased survival of said subject. In another related aspect, the incidence of organ failure or organ dysfunction, or organ damage, or a combination thereof, in a subject treated by the method, is reduced. In a further related aspect, the organ failure comprises acute multiple organ failure.

The present invention relates to methods of using a peptide of any of Formulas I-IV and II′-III′ as a pharmaceutical agent for the treatment and prevention of radiation and/or chemotherapy related injuries and/or afflictions, such as myelosuppression and decreased macrophage activity. The present invention relates to methods of using a peptide of any of Formulas I-IV and II′-III′ as a radioprotective agent. The peptides can also be used for the treatment of skin injury from UVB irradiation.

In one embodiment, peptides that are apelin receptor agonists are disclosed.

Such apelin agonist peptides of any one or more of the amino acid sequences set forth in any one of SEQ ID NO: 1-64 and 69-79 are disclosed.

An embodiment comprises a peptide of the amino acid sequence of Formula I:

(I)
(SEQ ID NO: 1)
X¹-R-X²-X³-X⁴-X⁵-X⁶-Q-X⁷-L-X⁸-X⁹

wherein X¹ is absent or if present is an amino acid having a polar side chain or a non-polar side chain; X² is an amino acid having a polar side chain or a non-polar side chain; X³ is absent or if present is one to three amino acids, each amino acid independently having a polar side chain or a non-polar side chain; X⁴ is an amino acid having a polar side chain or a non-polar side chain; X⁵ is an amino acid having a non-polar side chain; X⁶ is an amino acid having a polar side chain or a non-polar side chain; X⁷ is an amino acid having a polar side chain; X⁸ is an amino acid having a polar side chain; and X⁹ is absent or if present is one to three amino acids, each amino acid independently having a polar side chain or a non-polar side chain; or an analog of said peptide having a deletion, insertion or substitution of one, two, three, or four amino acids; or C-terminal acids or amides, or N-acetyl derivatives thereof; or pharmaceutically acceptable salts thereof.

An embodiment comprises a peptide of the amino acid sequence of Formula I wherein X³ is absent, or if present is —X¹²X¹¹X¹⁰—; wherein X¹⁰ is absent, or if present is an amino acid having a non-polar side chain; X¹¹ is absent, or if present is an amino acid having a non-polar side chain; and X¹² is an amino acid having a polar side chain or a non-polar side chain; or C-terminal acids or amides, or N-acetyl derivatives thereof; or pharmaceutically acceptable salts thereof.

An embodiment comprises a peptide of the amino acid sequence of Formula I wherein X⁹ is absent, or if present is —X¹³X¹⁴X¹⁵; wherein X¹³ is an amino acid having a non-polar side chain; X¹⁴ is absent, or if present is an amino acid having a non-polar side chain; and X¹⁵ is absent, or if present is an amino acid having a polar side chain; or C-terminal acids or amides, or N-acetyl derivatives thereof; or pharmaceutically acceptable salts thereof.

An embodiment comprises a peptide of the amino acid sequence of Formula I wherein X¹ is absent, or if present is selected from D, (dD), E, (dE), K, (dK), R, (dR), H, (dH), N, (dN), Q, (dQ), S, (dS), T, (dT), Y, (dY), C, (dC), G, A, (dA), V, (dV), L, (dL), I, (dl), F, (dF), W, (dW), P (dP), M and (dM); X² is selected from D, (dD), E, (dE), K, (dK), R, (dR), H, (dH), N, (dN), Q, (dQ), S, (dS), T, (dT), Y, (dY), C, (dC), G, A, (dA), V, (dV), L, (dL), I, (dl), F, (dF), W, (dW), P (dP), M and (dM); X³ is absent or if present is D, (dD), E, (dE), K, (dK), R, (dR), H, (dH), N, (dN), Q, (dQ), S, (dS), T, (dT), Y, (dY), C, (dC), G, A, (dA), V, (dV), L, (dL), I, (dl), F, (dF), W, (dW), P (dP), M, (dM) or —X¹²X¹¹X¹⁰—; X⁴ is an amino acid selected from D, (dD), E, (dE), K, (dK), R, (dR), H, (dH), N, (dN), Q, (dQ), S, (dS), T, (dT), Y, (dY), C, (dC), G, A, (dA), V, (dV), L, (dL), I, (dl), F, (dF), W, (dW), P (dP), M and (dM); X⁵ is an amino acid selected from G, A, (dA), V, (dV), L, (dL), I, (dl), F, (dF), W, (dW), P (dP), M and (dM); X⁶ is an amino acid selected from D, (dD), E, (dE), K, (dK), R, (dR), H, (dH), N, (dN), Q, (dQ), S, (dS), T, (dT), Y, (dY), C, (dC), G, A, (dA), V, (dV), L, (dL), I, (dl), F, (dF), W, (dW), P (dP), M and (dM); X⁷ is an amino acid selected from D, (dD), E, (dE), K, (dK), R, (dR), H, (dH), N, (dN), Q, (dQ), S, (dS), T, (dT), Y, (dY), C, and (dC); X⁸ is an amino acid selected from D, (dD), E, (dE), K, (dK), R, (dR), H, (dH), N, (dN), Q, (dQ), S, (dS), T, (dT), Y, (dY), C, and (dC); X⁹ is absent or if present is an amino acid independently selected from G, A, (dA), V, (dV), L, (dL), I, (dl), F, (dF), W, (dW), P (dP), M and (dM) or —X¹²X¹³X¹⁴; X¹⁰ is absent, or if present is an amino acid selected from G, A, (dA), V, (dV), L, (dL), I, (dl), F, (dF), W, (dW), P (dP), M and (dM); X¹¹ is absent, or if present is an amino acid selected from G, A, (dA), V, (dV), L, (dL), I, (dl), F, (dF), W, (dW), P (dP), M and (dM); X¹² is an amino acid selected from G, A, (dA), V, (dV), L, (dL), I, (dl), F, (dF), W, (dW), P (dP), M and (dM); X¹³ is an amino acid selected from G, A, (dA), V, (dV), L, (dL), I, (dl), F, (dF), W, (dW), P (dP), M and (dM); X¹⁴ is absent, or if present is an amino acid selected from G, A, (dA), V, (dV), L, (dL), I, (dl), F, (dF), W, (dW), P (dP), M and (dM); and X¹⁵ is absent, or if present is an amino acid selected from D, (dD), E, (dE), K, (dK), R, (dR), H, (dH), N, (dN), Q, (dQ), S, (dS), T, (dT), Y, (dY), C, and (dC); or C-terminal acids or amides, or N-acetyl derivatives thereof; or pharmaceutically acceptable salts thereof.

An embodiment comprises a peptide of the amino acid sequence of Formula I wherein X¹ is M, K, or absent; X² is R or Aib; X³ is absent or if present is M, E, -MMG-, -II(dA)-, -Nle-Nle-G- or -IIG-; X⁴ is M, E, I or Nle; X⁵ is V, A or G; X⁶ is F, Y, A or E; X⁷ is C, S or E; X⁸ is C, S or E; and X⁹ is -GL, -G(dA), -G(dA)K, -(dA)L, G or absent; or C-terminal acids or amides, or N-acetyl derivatives thereof; or pharmaceutically acceptable salts thereof.

An embodiment comprises a peptide of the amino acid sequence of Formula I wherein X¹ is M, K, or absent; X² is R or Aib; X³ is absent or if present is M, E, -MMG-, -LLG-, -II(dA)-, -Nle-Nle-G- or -IIG-; X⁴ is M, E, L, I or Nle; X⁵ is V, A or G; X⁶ is F, Y, A or E; X⁷ is C, S or E; X⁸ is C, S or E; and X⁹ is -GL, -G(dA), -G(dA)K, -(dA)L, G or absent; or C-terminal acids or amides, or N-acetyl derivatives thereof; or pharmaceutically acceptable salts thereof.

An embodiment comprises a peptide of the amino acid sequence of Formula I wherein X⁷ is S; or C-terminal acids or amides, or N-acetyl derivatives thereof; or pharmaceutically acceptable salts thereof.

An embodiment comprises a peptide of the amino acid sequence of Formula I wherein X³ is absent or if present is -LLG-; X⁴ is L; X⁵ is V; and/or X⁸ is C or E; or C-terminal acids or amides, or N-acetyl derivatives thereof; or pharmaceutically acceptable salts thereof.

An embodiment comprises a peptide of the amino acid sequence of Formula I wherein X¹ is (PEG12)-K, and/or wherein X⁹ is -G(dA)-K(PEG12).

An embodiment comprising a peptide of the amino acid sequence of Formula II:

(II)
(SEQ ID NO: 64)
X¹⁶-M-M-G-M-X¹⁷

wherein X¹⁶ is absent or if present is R— or R—R—; and X¹⁷ is absent or if present is selected from —V, —VF, -VFQ, -VFQS, -VFQSL, and -VFQSLCG(dA); C-terminal acids or amides, or N-acetyl derivatives thereof; or pharmaceutically acceptable salt thereof.

An embodiment comprises a peptide of the amino acid sequence of Formula II wherein X¹⁶ is R— or RR—; and X¹⁷ is selected from VF, -VFQ, -VFQS, -VFQSL, and -VFQSLCG(dA); C-terminal acids or amides, or N-acetyl derivatives thereof; or pharmaceutically acceptable salt thereof.

An embodiment comprising a peptide of the amino acid sequence of Formula II′:

X¹⁶-M-M-G-M-X¹⁷ (II′) (SEQ ID NO: 79)

wherein X¹⁶ is absent or if present is R—, R-Aib, or R—R—; and X¹⁷ is absent or if present is selected from —V, —VF, -VFQ, -VFQS, -VFQSL, and -VFQSLCG(dA); C-terminal acids or amides, or N-acetyl derivatives thereof; or pharmaceutically acceptable salt thereof.

An embodiment comprises a peptide of the amino acid sequence of Formula II′ wherein X¹⁶ is R-Aib; C-terminal acids or amides, or N-acetyl derivatives thereof; or pharmaceutically acceptable salt thereof.

An embodiment comprises an amino acid sequence selected from MRRMMGMVFQCLCGL (SEQ ID NO: 7); RRMMGMVFQCLCG(dA) (SEQ ID NO: 8); RRMMGMVYQCLCG(dA) (SEQ ID NO: 10); RRMMGMVAQCLCG(dA) (SEQ ID NO: 11); RRMMGMVFQELCG(dA) (SEQ ID NO: 13); RRMMGMVFQCLEG(dA) (SEQ ID NO: 14); RRMMGMVFQSLCG(dA) (SEQ ID NO: 15); RR(Nle)(Nle)G(Nle)VFQCLCG(dA) (SEQ ID NO: 18); (PEG12)KRRMMGMVFQCLCG(dA) (SEQ ID NO: 20); RRMMGMVFQCLCG(dA)K(PEG12) (SEQ ID NO: 21); RRMVYQCLCG(dA) (SEQ ID NO: 22); RRMMGMVAQCLEG(dA) (SEQ ID NO: 30); R(Aib)MMGMVFQSLCG(dA) (SEQ ID NO: 34); (PEG12)KRRMMGMVFQSLCG(dA) (SEQ ID NO: 36); (PEG12)KRRLLGLVFQSLCG(dA) (SEQ ID NO: 37); (PEG12)KRRIIGIVFQCLCG(dA) (SEQ ID NO: 42); RRIIGIVFQSLCG(dA) (SEQ ID NO: 43); or pharmaceutically acceptable salt thereof.

An embodiment comprises an amino acid sequence selected from MRRMMGMVFQCLCGL (SEQ ID NO: 7); RRMMGMVFQSLCG(dA) (SEQ ID NO: 15); (PEG12)KRRMMGMVFQSLCG(dA) (SEQ ID NO: 36); (PEG12)KRRLLGLVFQSLCG(dA) (SEQ ID NO: 37); RRIIGIVFQSLCG(dA) (SEQ ID NO: 43); or pharmaceutically acceptable salt thereof.

An embodiment comprises an amino acid sequence selected from MRRMMGMVFQCLCGL (SEQ ID NO: 7); RRMMGMVFQSLCG(dA) (SEQ ID NO: 15); (PEG12)KRRMMGMVFQSLCG(dA) (SEQ ID NO: 36); RRIIGIVFQSLCG(dA) (SEQ ID NO: 43); or pharmaceutically acceptable salt thereof.

An embodiment comprises treating a disease or disorder of the invention in a subject in need thereof, comprising administering to the subject a peptide comprising an amino acid sequence of Formula III:

(III)
(SEQ ID NO: 69)
X¹⁸-X¹⁹-X²⁰-X²¹ V-X²²-Q-X²³ I-X²⁴-G-X²⁵

wherein X¹⁸ is absent or if present is M or K; X¹⁹ is R or Aib; X²⁰ is absent or if present is -M-M-G- or Nle-Nle-G-; X²¹ is M or Nle; X²² is F, A or Y; X²³ is S, E or C; X²⁴ is E or C; X²⁵ is L, dA or -dA-K; C-terminal acids or amides, or N-acetyl derivatives thereof; or pegylated derivatives thereof; or pharmaceutically acceptable salt thereof.

An embodiment comprises treating a disease or disorder of the invention in a subject in need thereof, comprising administering to the subject a peptide comprising an amino acid sequence of Formula III′:

(III′)
(SEQ ID NO: 78)
X¹⁸-R-X¹⁹-X²⁰-X²¹ V-X²²-Q-X²³ L-X²⁴-G-X²⁵

wherein X¹⁸ is absent or if present is M or K; X¹⁹ is R or Aib; X²⁰ is absent or if present is -M-M-G- or Nle-Nle-G-; X²¹ is M or Nle; X²² is F, A or Y; X²³ is S, E or C; X²⁴ is E or C; X²⁵ is L, dA or -dA-K; C-terminal acids or amides, or N-acetyl derivatives thereof; or pegylated derivatives thereof; or pharmaceutically acceptable salt thereof.

An embodiment comprises sequences wherein X²⁵ is dA; C-terminal acids or amides, or N-acetyl derivatives thereof; or pegylated derivatives thereof; or pharmaceutically acceptable salt thereof.

An embodiment comprises sequences wherein X¹⁹ is R; X²⁰ is absent or if present is -M-M-G-; and X²¹ is M; C-terminal acids or amides, or N-acetyl derivatives thereof; or pegylated derivatives thereof; or pharmaceutically acceptable salt thereof.

An embodiment comprises sequences wherein X²² is F; and X²³ is C; C-terminal acids or amides, or N-acetyl derivatives thereof; or pegylated derivatives thereof; or pharmaceutically acceptable salt thereof.

An embodiment comprises sequences wherein the peptide or analog comprises or consists of an amino acid sequence selected from MRRMMGMVFQCLCGL (SEQ ID NO: 7); RRMMGMVFQSLCG(dA) (SEQ ID NO: 15); and (PEG12)KRRMMGMVFQSLCG(dA) (SEQ ID NO: 36); or pharmaceutically acceptable salt thereof.

An embodiment comprises sequences wherein the peptide or analog comprises or consists of an amino acid sequence selected from (PEG12)RRMMGMVFQSLCG(dA) (SEQ ID NO: 71); and (K(PEG12))RRMMGMVFQSLCG(dA) (SEQ ID NO: 72); or pharmaceutically acceptable salt thereof.

An embodiment comprises a method of treating a disease or disorder of the invention comprising administering to the subject a peptide comprising either an amino acid sequence of Formula IV:

(IV)
(SEQ ID NO: 70)
X²⁶-RR-X²⁷-X²⁸ G-X²⁹-VFQ-X³⁰-LCG-(dA)

wherein X²⁶ is absent or if present is K; X²⁷ is L or I; X²⁸ is L or I; X²⁹ is L or I; and X³⁰ is S or C; C-terminal acids or amides, or N-acetyl derivatives thereof; or pegylated derivatives thereof; or pharmaceutically acceptable salt thereof.

An embodiment comprises sequences wherein X³⁰ is S; C-terminal acids or amides, or N-acetyl derivatives thereof; or pegylated derivatives thereof; or pharmaceutically acceptable salt thereof.

An embodiment comprises sequences wherein X²⁷ is L; X²⁸ is L; and/or X²⁹ is L; C-terminal acids or amides, or N-acetyl derivatives thereof; or pegylated derivatives thereof; or pharmaceutically acceptable salt thereof.

An embodiment comprises sequences wherein the peptide or analog comprises or consists of an amino acid sequence selected from (PEG12)KRRLLGLVFQSLCG(dA) (SEQ ID NO: 37); or RRIIGIVFQSLCG(dA) (SEQ ID NO: 43); (SEQ ID NO: 36); or pharmaceutically acceptable salt thereof.

An embodiment comprises sequences wherein the peptide or analog comprises or consists of an amino acid sequence selected from (K(PEG12))RRLLGLVFQSLCG(dA) (SEQ ID NO: 73); (PEG12)RRLLGLVFQSLCG(dA) (SEQ ID NO: 74); (PEG12)KRRIIGIVFQSLCG(dA) (SEQ ID NO: 75); (K(PEG12))RRIIGIVFQSLCG(dA) (SEQ ID NO: 76); and (PEG12)RRIIGIVFQSLCG(dA) (SEQ ID NO: 77); or pharmaceutically acceptable salt thereof.

In some embodiments, a peptide is represented by the peptides listed in Table 1.

TABLE 1

| Sequence | SEQ ID NO: |
|---|---|
| MRRIIGIVFQCLCGL | 2 |
| RRIIGIVFQCLCGL | 3 |
| RRIIGIVFQCLCG | 4 |
| RRIIGIVFQCLC | 5 |
| RRIIGIVFQCLC(dA)L | 6 |
| MRRMMGMVFQCLCGL | 7 |
| RRMMGMVFQCLCG(dA) | 8 |
| RRII(dA)IVFQCLC(dA)L | 9 |
| RRMMGMVYQCLCG(dA) | 10 |
| RRMMGMVAQCLCG(dA) | 11 |
| RRMMGMVEQCLCG(dA) | 12 |
| RRMMGMVFQELCG(dA) | 13 |
| RRMMGMVFQCLEG(dA) | 14 |
| RRMMGMVFQSLCG(dA) | 15 |
| RRMMGMVFQCLSG(dA) | 16 |
| RRMMGMVFQSLSG(dA) | 17 |
| RR(Nle)(Nle)G(Nle)VFQCLCG(dA) | 18 |
| RRMVFQCLCG(dA) | 19 |
| (PEG12)KRRMMGMVFQCLCG(dA) | 20 |
| RRMMGMVFQCLCG(dA)K(PEG12) | 21 |
| RRMVYQCLCG(dA) | 22 |
| RRMVFQCLEG(dA) | 23 |
| RRMVYQCLEG(dA) | 24 |
| RREMVYQCLCG(dA) | 25 |
| RREMVYQCLEG(dA) | 26 |
| RRMAYQCLEG(dA) | 27 |
| RRMGYQCLEG(dA) | 28 |
| RRMMGMVYQCLEG(dA) | 29 |
| RRMMGMVAQCLEG(dA) | 30 |
| RRMEVYQCLCG(dA) | 31 |
| RRMEVYQCLEG(dA) | 32 |
| RRLLGLVFQSLCG(dA) | 33 |
| R(Aib)MMGMVFQSLCG(dA) | 34 |
| R(Aib)LLGLVFQSLCG(dA) | 35 |
| (PEG12)KRRMMGMVFQSLCG(dA) | 36 |
| (PEG12)KRRLLGLVFQSLCG(dA) | 37 |
| RRMMGMVEQSLCG(dA) | 38 |
| RRMMGMVFQSLEG(dA) | 39 |
| RRLLGLVEQSLCG(dA) | 40 |
| RRLLGLVFQSLEG(dA) | 41 |
| (PEG12)KRRIIGIVFQCLCG(dA) | 42 |
| RRIIGIVFQSLCG(dA) | 43 |
| MMGMV | 44 |
| MMGMVF | 45 |
| MMGMVFQ | 46 |
| MMGMVFQS | 47 |
| MMGMVFQSL | 48 |
| MMGMVFQSLCG(dA) | 49 |
| RMMGMVF | 50 |
| RMMGMVFQ | 51 |
| RMMGMVFQS | 52 |
| RMMGMVFQSL | 53 |
| RMMGMVFQSLCG(dA) | 54 |
| RRMMGM | 55 |
| RRMMGMV | 56 |
| RRMMGMVF | 57 |
| RRMMGMVFQ | 58 |
| RRMMGMVFQS | 59 |
| RRMMGMVFQSL | 60 |
| Acetyl-RRMMGMVFQSLCG(dA) | 61 |
| RRMMGMVFQSLCG(dA)-Amide | 62 |
| Acetyl-RRMMGMVFQSLCG(dA)-Amide | 63 |
| (PEG12)RRMMGMVFQSLCG(dA) | 71 |
| (K(PEG12))RRMMGMVFQSLCG(dA) | 72 |
| (K(PEG12))RRLLGLVFQSLCG(dA) | 73 |
| (PEG12)RRLLGLVFQSLCG(dA) | 74 |
| (PEG12)KRRIIGIVFQSLCG(dA) | 75 |
| (K(PEG12))RRIIGIVFQSLCG(dA) | 76 |
| (PEG12)RRIIGIVFQSLCG(dA) | 77 |

In some embodiments, a peptide is an acetate, or hydrochloric salt of a peptide listed in Table 1.

The peptides can be prepared as described in U.S. Provisional Application No. 62/887,049, incorporated herein by reference. In exemplary embodiments, the peptide or peptide derivative is a PEG, acetyl, biotin or fatty acid derivative thereof. In exemplary embodiments, the peptide derivative includes PEG12.

In exemplary aspects, the peptide or peptide analog of the present disclosure are agonists of apelin receptor. In exemplary aspects, the level of agonism is at least or about 30%, relative to a control. In exemplary aspects, the level of agonism is at least or about 40%, at least or about 50%, at least or about 60%, at least or about 70%, at least or about 80%, at least or about 90%, relative to a control. In exemplary aspects, the level of apelin receptor agonism is greater than 90%, relative to a control. Suitable methods of assaying apelin receptor agonism levels are known, a few exemplary methods of which are described here in Examples 2-3 and 9-11. In exemplary aspects, the peptide or peptide analog of the present disclosure acts as agonists of apelin receptor, as assayed by a method described in one of Examples 2-3 and 9-11. In exemplary aspects, the peptide or peptide analog of the present disclosure acts as agonists of apelin receptor, as assayed by a single dose assay described in one of Examples 2-3 and 9-11.

In exemplary aspects, the peptide or peptide analog of the present disclosure exhibits at least a 10% stability in mouse plasma for 60 minutes at 37 degrees Celsius. In other words, at least 10% of the starting assay amount of the peptide or peptide analog is present in an intact state (e.g., not degraded, cleaved, etc.) after being incubated in mouse plasma for 60 minutes at 37 degrees Celsius. In exemplary aspects, the peptide or peptide analog exhibits at least a 20% stability, at least or about a 30% stability, at least or about a 40% stability, at least or about a 50% stability, at least or about a 60% stability, at least or about a 70% stability, at least or about a 80% stability, or at least or about a 90% stability, in plasma for 60 minutes at 37 degrees Celsius. Suitable methods of assaying the stability of peptides in plasma (included mouse plasma) are known in the art. In exemplary aspects, the peptide or peptide analog of the present disclosure exhibits at least a 10% stability in mouse plasma for 60 minutes at 37 degrees Celsius. In exemplary aspects, the peptide or peptide analog of the present disclosure exhibits at least a 10% stability in mouse plasma for 60 minutes at 37 degrees Celsius, as assayed by a single peptide dose/concentration assay.

### Peptide Length

In exemplary embodiments, the peptide or peptide analog of the present disclosure is a peptide or peptide analog comprising at least four amino acids connected via peptide bonds or other covalent linkages, as described herein. In exemplary aspects, the peptide or peptide analog is about 4 to about 50 amino acids in length. All integer subranges of 4 to 50 amino acids are specifically contemplated for peptides herein. In exemplary aspects, the peptide or peptide analog is about 5 to about 35 amino acids in length, about 5 to about 30 amino acids in length, about 5 to about 25 amino acids in length, or about 5 to about 20 amino acids in length. In exemplary aspects, the peptide or peptide analog is about 6 to about 35 amino acids in length, about 7 to about 30 amino acids in length, about 6 to about 25 amino acids in length, or about 6 to about 20 amino acids in length. In exemplary aspects, the peptide or peptide analog is about 7 to about 35 amino acids in length, about 7 to about 30 amino acids in length, about 7 to about 25 amino acids in length, or about 7 to about 20 amino acids in length. In exemplary aspects, the peptide or peptide analog is about 8 to about 35 amino acids in length, about 8 to about 30 amino acids in length, about 8 to about 25 amino acids in length, or about 8 to about 20 amino acids in length. In exemplary aspects, the peptide is about 8 to about 17 or 18 or about 9 to about 16 or 17 amino acids in length. In exemplary aspects, the peptide is about 10 to about 17 or about 12 to about 16 or 17 or about 14 to about 16 amino acids in length. In some embodiments, the peptide is a 5-mer, 6-mer, 7-mer, 8-mer, 9-mer-10-mer, 11-mer, 12-mer, 13-mer, 14-mer, 15-mer, 16-mer, 17-mer, 18-mer, 19-mer, or 20-mer.

### Peptide Modifications

Peptides of the disclosure include peptides that have been modified in any way and for any reason, for example, to: (1) reduce susceptibility to proteolysis, (2) alter binding affinities, and (3) confer or modify other physicochemical or functional properties. For example, single or multiple amino acid substitutions (e.g., equivalent, conservative or non-conservative substitutions, deletions or additions) may be made in a sequence. In exemplary aspects, the peptide or peptide analog of the present disclosure comprises a sequence listed in Table 1, or a modified sequence thereof. In exemplary embodiments of the present disclosure, the peptide or peptide analog is lipidated (e.g., myristoylated, palmitoylated, linked to a C₇-C₂₀ lipid moiety), glycosylated, amidated, carboxylated, phosphorylated, esterified, acylated, acetylated, cyclized, pegylated (e.g., linked to a 5-20 kDa PEG, linked to a 5 kDa PEG, 12 kDa PEG, 20 kDa PEG) to or converted into an acid addition salt and/or optionally dimerized or polymerized, or conjugated, as further described herein. PEG in sizes of 200-4600 mol wt also would be of use for modifying the peptides of the current invention. PEG that are linear, branched and star geometries also would be of use for modifying the peptides of the current invention. PEG600 is also known as PEG12. In exemplary embodiments of the present disclosure, the peptide or peptide analog is acetylated at the N-terminus, amidated at the C-terminus, and/or phosphorylated on a Tyr residue. In exemplary aspects, the peptide or peptide analog is linked to a lipid moiety at the N-terminus or side chain of an internal residue. In exemplary aspects, the peptide or peptide analog is directly linked to a lipid moiety. In exemplary aspects, the peptide or peptide analog is indirectly linked to a lipid moiety. For example, the lipid moiety may be attached to the peptide via a linker. The linker may be an amino acid. In exemplary aspects, the lipid moiety is attached to a Lys residue of the peptide or peptide analog via a Glu residue optionally attached via the epsilon amine. Examples of modified peptides of the invention are found in Table 1.

In some embodiments, peptides disclosed herein comprise a sequence having at least 66% sequence identity to any one of amino acid sequences SEQ ID NO: 1-64 and 69-79. In certain embodiments, the % identity is selected from, e.g., at least 70%, at least 75%, at least 80%, at least 85%, at least 90%, or at least 95%, or more sequence identity to a given sequence. In certain embodiments, the % identity is in the range of, e.g., about 65% to about 70%, about 70% to about 80%, about 80% to about 85%, about 85% to about 90%, or about 90% to about 95%; %; between about 70% and about 80%, between about 80% and about 90% and between about 90% and about 99% sequence identity.

In certain embodiments, the peptide comprises a sequence having at least 66% sequence identity to any one of amino acid sequences SEQ ID NO:1-64 and 69-79. In certain embodiments, the % identity is selected from, e.g., at least 70%, at least 75%, at least 80%, at least 85%, at least 90%, or at least 95%, or more sequence identity to a given sequence. In certain embodiments, the % identity is in the range of, e.g., about 65% to about 70%, about 70% to about 80%, about 80% to about 85%, about 85% to about 90%, or about 90% to about 95%; %; between about 70% and about 80%, between about 80% and about 90% and between about 90% and about 99% sequence identity, but does not comprise the sequence set forth in SEQ ID NO: 2.

Peptides of the disclosure include peptides that have been modified in any way and for any reason, for example, to: (1) reduce susceptibility to proteolysis, (2) alter binding affinities, and (3) confer or modify other physicochemical or functional properties. For example, single or multiple amino acid substitutions (e.g., equivalent, conservative or non-conservative substitutions, deletions or additions) may be made in a sequence.

A conservative amino acid substitution refers to the substitution in a peptide of an amino acid with a functionally similar amino acid having similar properties, e.g., size, charge, hydrophobicity, hydrophilicity, and/or aromaticity. The following six groups each contain amino acids that are conservative substitutions for one another are found in Table 2.

TABLE 2

| | |
|---|---|
| i. | Alanine (A), Serine (S), and Threonine (T) |
| ii. | Aspartic acid (D) and Glutamic acid (E) |
| iii. | Asparagine (N) and Glutamine (Q) |
| iv. | Arginine (R) and Lysine (K) |
| v. | Isoleucine (I), Leucine (L), Methionine (M), and Valine (V) |
| vi. | Phenylalanine (F), Tyrosine (Y), and Tryptophan (W) |

Additionally, within the meaning of the term “equivalent amino acid substitution” as applied herein, one amino acid may be substituted for another, in one embodiment, within the groups of amino acids indicated herein below:
1. Amino acids with polar side chains (Asp, Glu, Lys, Arg, His, Asn, Gln, Ser, Thr, Tyr, and Cys,)
2. Amino acids with small nonpolar or slightly polar residues (Ala, Ser, Thr, Pro, Gly);
3. Amino acids with non-polar side chains (Gly, Ala, Val, Leu, Ile, Phe, Trp, Pro, and Met)
4. Amino acids with large, aliphatic, nonpolar residues (Met, Leu, Ile, Val, Cys, Norleucine (Nle), homocysteine)
5. Amino acids with aliphatic side chains (Gly, Ala Val, Leu, Ile)
6. Amino acids with cyclic side chains (Phe, Tyr, Trp, His, Pro)
7. Amino acids with aromatic side chains (Phe, Tyr, Trp)
8. Amino acids with acidic side chains (Asp, Glu)
9. Amino acids with basic side chains (Lys, Arg, His)
10. Amino acids with amide side chains (Asn, Gln)
11. Amino acids with hydroxy side chains (Ser, Thr)
12. Amino acids with sulphur-containing side chains (Cys, Met),
13. Neutral, weakly hydrophobic amino acids (Pro, Ala, Gly, Ser, Thr)
14. Hydrophilic, acidic amino acids (Gln, Asn, Glu, Asp), and
15. Hydrophobic amino acids (Leu, Ile, Val).

In some embodiments, the amino acid substitution is not a conservative amino acid substitution, e.g., is a non-conservative amino acid substitution. This class generally includes corresponding D-amino acids, homo-amino acids, N-alkyl amino acids, beta amino acids and other unnatural amino acids. The non-conservative amino acid substitutions still fall within the descriptions identified for the equivalent amino acid substitutions above [e.g. polar, nonpolar, etc.]. Examples of non-conservative amino acids are provided below.

Non limiting examples for alanine non-conservative amino acids are: D-alanine [Dala, (dA), a], N-Acetyl-3-(3,4-dimethoxyphenyl)-D-alanine, N-Me-D-Ala-OH, N-Me-Ala-OH, H-β-Ala-β-naphthalene, L-(−)-2-Amino-3-ureidopropionic acid, (R)-(+)-α-Allylalanine, (S)-(−)-α-Allylalanine, D-2-Aminobutyric acid, L-2-Aminobutyric acid, DL-2-Aminobutyric acid, 2-Aminoisobutyric acid, α-Aminoisobutyric acid, (S)-(+)-2-Amino-4-phenylbutyric acid ethyl ester, Benzyl α-aminoisobutyrate, Abu-OH, Aib-OH, β-(9-anthryl)-Ala-OH, β-(3-benzothienyl)-Ala-OH, β-(3-benzothienyl)-D-Ala-OH, Cha-OH, Cha-OMe, β-(2-furyl)-Ala-OH, β-(2-furyl)-D-Ala-OH, β-iodo-Ala-OBzI, β-iodo-D-Ala-OBzI, 3-iodo-D-Ala-OMe, β-iodo-Ala-OMe, 1-Nal-OH, D-1-Nal-OH, 2-Nal-OH, D-2-Nal-OH, (R)-3-(2-naphthyl)-β-Ala-OH, (S)-3-(2-naphthyl)-β-Ala-OH, β-phenyl-Phe-OH, 3-(2-pyridyl)-Ala-OH, 3-(3-pyridyl)-Ala-OH, 3-(3-pyridyl)-D-Ala-OH, (S)-3-(3-pyridyl)-β-Ala-OH, 3-(4-pyridyl)-Ala-OH, 3-(4-pyridyl)-D-Ala-OH, β-(2-quinolyl)-Ala-OH, 3-(2-quinolyl)-DL-Ala-OH, 3-(3-quinolyl)-DL-Ala-OH, 3-(2-quinoxalyl)-DL-Ala-OH, β-(4-thiazolyl)-Ala-OH, R-(2-thienyl)-Ala-OH, β-(2-thienyl)-D-Ala-OH, R-(3-thienyl)-Ala-OH, β-(3-thienyl)-D-Ala-OH, 3-Chloro-D-alanine methyl ester, N-[(4-Chlorophenyl)sulfonyl]-3-alanine, 3-Cyclohexyl-D-alanine, 3-Cyclopentyl-DL-alanine, (−)-3-(3,4-Dihydroxyphenyl)-2-methyl-L-alanine, 3,3-Diphenyl-D-alanine, 3,3-Diphenyl-L-alanine, N—[(S)-(+)-1-(Ethoxycarbonyl)-3-phenylpropyl]-L-alanine, N-[1-(S)-(+)-Ethoxycarbonyl-3-phenylpropyl]-L-alanyl carboxyanhydride, N-(3-fluorobenzyl)alanine, N-(3-Indolylacetyl)-L-alanine, Methyl (RS)-2-(aminomethyl)-3-phenylpropionate, 3-(2-Oxo-1,2-dihydro-4-quinolinyl)alanine, 3-(1-Pyrazolyl)-L-alanine, 3-(2-Pyridyl)-D-alanine, 3-(2-Pyridyl)-L-alanine, 3-(3-Pyridyl)-L-alanine, 3-(4-Pyridyl)-D-alanine, 3-(4-Pyridyl)-L-alanine, 3-(2-Quinolyl)-DL-alanine, 3-(4-Quinolyl)-DL-alanine, D-styrylalanine, L-styrylalanine, 3-(2-Thienyl)-L-alanine, 3-(2-Thienyl)-DL-alanine, 3-(2-Thienyl)-DL-alanine, 3,3,3-Trifluoro-DL-alanine, N-Methyl-L-alanine, 3-Ureidopropionic acid, Aib-OH, Cha-OH, Dehydro-Ala-OMe, dehydro-Ala-OH, D-2-Nal-OH, β-Ala-ONp, β-Homoala-OH, β-D-Homoala-OH, β-Alanine, β-Alanine ethyl ester, β-Alanine methyl ester, (S)-diphenyl-β-Homoala-OH, (R)-4-(4-pyridyl)-β-Homoala-OH, (S)-4-(4-pyridyl)-β-Homoala-OH, β-Ala-OH, (S)-diphenyl-β-Homoala-OH, L-β-Homoalanine, (R)-4-(3-pyridyl)-β-Homoala-OH, α-methyl-α-naphthylalanine [Manap], N-methyl-cyclohexylalanine [Nmchexa], cyclohexylalanine [Chexa], N-methyl-cyclopentylalanine [Nmcpen], cyclopentylalanine [Cpen], N-methyl-α-naphthylalanine [Nmanap], α-naphthylalanine [Anap], L-N-methylalanine [Nmala], D-N-methylalanine [Dnmala], α-methyl-cyclohexylalanine [Mchexa], α-methyl-cyclopentylalanine [Mcpen]. Each possibility represents a separate embodiment.

Non limiting examples for arginine non-conservative amino acids are: homoarginine (hArg), N-methyl arginine (NMeArg), citruline, 2-amino-3-guanidinopropionic acid, N-iminoethyl-L-ornithine, Nω-monomethyl-L-arginine, Nω-nitro-L-arginine, D-arginine, 2-amino-3-ureidopropionic acid, Nω,ω-dimethyl-L-arginine, Nω-Nitro-D-arginine, L-α-methylarginine [Marg], D-α-methylarginine [Dmarg], L-N-methylarginine [Nmarg], D-N-methylarginine [Dnmarg], β-Homoarg-OH, L-Homoarginine, N-(3-guanidinopropyl)glycine [Narg], and D-arginine [Darg, (dR), r]. Each possibility represents a separate embodiment.

Non limiting examples for asparagine non-conservative amino acids are: L-α-methylasparagine [Masn], D-α-methylasparagine [Dmasn], L-N-methylasparagine [Nmasn], D-N-methylasparagine [Dnmasn], N-(carbamylmethyl)glycine [Nasn] and D-asparagine [Dasn, (dN), n]. Each possibility represents a separate embodiment.

Non limiting examples for aspartic acid non-conservative amino acids are: L-α-methylaspartate [Masp], D-α-methylaspartate [Dmasp], L-N-methylaspartic acid [Nmasp], D-N-methylasparatate [Dnmasp], N-(carboxymethyl)glycine [Nasp] and D-aspartic acid [Dasp, (dD), d]. Each possibility represents a separate embodiment.

Non limiting examples for cysteine non-conservative amino acids are: L-Cysteic acid, L-Cysteinesulfinic acid, D-Ethionine, S-(2-Thiazolyl)-L-cysteine, DL-Homocysteine, L-Homocysteine, L-Homocystine, L-α-methylcysteine [Mcys], D-α-methylcysteine [Dmcys], L-N-methylcysteine [Nmcys], D-N-methylcysteine [Dnmcys], N-(thiomethyl)glycine [Ncys] and D-cysteine [Dcys, (dC), c]. Each possibility represents a separate embodiment.

Non limiting examples for glutamic acid non-conservative amino acids are: γ-Carboxy-DL-glutamic acid, 4-Fluoro-DL-glutamic acid, β-Glutamic acid, L-β-Homoglutamic acid, L-α-methylglutamate [Mglu], D-α-methyl glutamic acid [Dmglu], L-N-methylglutamic acid [Nmglu], D-N-methylglutamate [Dnmglu], N-(2-carboxyethyl)glycine [Nglu], and D-glutamic acid [Dglu, (dE), e]. Each possibility represents a separate embodiment.

Non limiting examples for glutamine non-conservative amino acids are: Cit-OH, D-Citrulline, Thio-L-citrulline, β-Gln-OH, L-β-Homoglutamine, L-α-methylglutamine [Mgln], D-α-methylglutamine [Dmgln], L-N-methylglutamine [Nmgln], D-N-methylglutamine [Dnmgln], N-(2-carbamylethyl)glycine [Ngln], and D-glutamine [Dgln, (dQ), q]. Each possibility represents a separate embodiment.

Non limiting examples for glycine non-conservative amino acids are: tBu-Gly-OH, D-Allylglycine, N-[Bis(methylthio)methylene]glycine methyl ester, Chg-OH, D-Chg-OH, D-cyclopropylglycine, L-cyclopropylglycine, (R)-4-fluorophenylglycine, (S)-4-fluorophenylglycine, iminodiacetic acid, (2-indanyl)-Gly-OH, (±)-α-phosphonoglycine trimethyl ester, D-propargylglycine, propargyl-Gly-OH, (R)-2-thienylglycine, (S)-2-thienylglycine, (R)-3-thienylglycine, (S)-3-thienylglycine, 2-(4-trifluoromethyl-phenyl)-DL-glycine, (2S,3R,4S)-α-(Carboxycyclopropyl)glycine, N-(Chloroacetyl)glycine ethyl ester, (S)-(+)-2-chlorophenylglycine methyl ester, N-(2-chlorophenyl)-N-(methylsulfonyl)glycine, D-α-Cyclohexylglycine, L-α-Cyclopropylglycine, Di-tert-butyl-iminodicarboxylate, Ethyl acetamidocyanoacetate, N-(2-fluorophenyl)-N-(methylsulfonyl) glycine, N-(4-fluorophenyl)-N-(methylsulfonyl)glycine, N-(2-Furfurylideneacetyl)glycine methyl ester, N-(2-Furoyl)glycine, N-(2-Hydroxyethyl)iminodiacetic acid, N-(4-Hydroxyphenyl)glycine, Iminodiacetic acid, N-Lauroylsarcosine sodium salt, L-α-Neopentylglycine, N-(Phosphonomethyl)glycine, D-Propargylglycine, L-C-Propargylglycine, Sarcosine, N,N-Dimethylglycine, N,N-Dimethylglycine ethyl ester, D-Chg-OH, α-Phosphonoglycine trimethyl ester, N-cyclobutylglycine [Ncbut], L-α-methylethylglycine [Metg], N-cycloheptylglycine [Nchep], L-α-methyl-1-butylglycine [Mtbug], N-methylglycine [Nmgly], L-N-methyl-ethylglycine [Nmetg], L-ethylglycine [Etg], L-N-methyl-t-butylglycine [Nmtbug], L-t-butylglycine [Tbug], N-cyclohexylglycine [Nchex], N-cyclodecylglycine [Ncdec], N-cyclododecylglycine [Ncdod], N-cyclooctylglycine [Ncoct], N-cyclopropylglycine [Ncpro], N-cycloundecylglycine [Ncund], N-(2-aminoethyl)glycine [Naeg], N—(N-(2,2-diphenylethyl) diphenylethyl)glycine [Nnbhm], N-(2,2-carbamylmethyl-glycine [Nbhm], N—(N-(3,3-diphenylpropyl) diphenylpropyl)glycine [Nnbhe] and N-(3,3-carbamylmethyl-glycine [Nbhe]. Each possibility represents a separate embodiment.

Non limiting examples for histidine non-conservative amino acids are: L-α-methylhistidine [Mhis], D-α-methylhistidine [Dmhis], L-N-methylhistidine [Nmhis], D-N-methylhistidine [Dnmhis], N-(imidazolylethyl)glycine [Nhis], and D-histidine [Dhis, (dH), h]. Each possibility represents a separate embodiment.

Non limiting examples for isoleucine non-conservative amino acids are: N-Methyl-L-isoleucine [Nmile], N-(3-Indolylacetyl)-L-isoleucine, allo-Ile-OH, D-allo-lsoleucine, L-β-Homoisoleucine, L-α-methylisoleucine [Mile], D-α-methylisoleucine [Dmile], D-N-methylisoleucine [Dnmile], N-(1-methylpropyl)glycine [Nile], and D-isoleucine [Dile, (dD), i]. Each possibility represents a separate embodiment.

Non limiting examples for leucine non-conservative amino acids are: D-leucine [Dleu, (dL), I]. Cycloleucine, DL-leucine, N-Formyl-Leu-OH, D-tert-Leucine, L-tert-Leucine, DL-tert-Leucine, L-tert-Leucine methyl ester, 5,5,5-Trifluoro-DL-leucine, D-β-Leu-OH, L-β-Leucine, DL-β-Leucine, L13-Homoleucine, DL-β-Homoleucine, L-N-methyl-leucine [Nmleu], D-N-methyl-leucine [Dnmleu], L-α-methyl-leucine [Mleu], D-α-methyl-leucine [Dmleu], N-(2-methylpropyl)glycine [Nleu], D-leucine [Dleu, I], D-Norleucine, L-Norleucine, DL-Norleucine, L-N-methylnorleucine [Nmnle] and L-norleucine [Nle]. Each possibility represents a separate embodiment.

Non limiting examples for lysine non-conservative amino acids are: DL-5-Hydroxylysine, (5R)-5-Hydroxy-L-lysine, β-Lys-OH, L13-Homolysine, L-α-methyl-lysine [Mlys], D-α-methyl-lysine [Dmlys], L-N-methyl-lysine [Nmlys], D-N-methyl-lysine [Dnmlys], N-(4-aminobutyl)glycine [Nlys], and D-lysine [Dlys, (dK), k]. Each possibility represents a separate embodiment.

Non limiting examples for methionine non-conservative amino acids are: L-β-Homomethionine, DL-β-Homomethionine, L-α-methylmethionine [Mmet], D-α-methylmethionine [Dmmet], L-N-methylmethionine [Nmmet], D-N-methylmethionine [Dnmmet], N-(2-methylthioethyl)glycine [Nmet], and D-methionine [Dmet, (dM), m]. Each possibility represents a separate embodiment.

Non limiting examples for phenylalanine non-conservative amino acids are: N-Acetyl-2-fluoro-DL-phenylalanine, N-Acetyl-4-fluoro-DL-phenylalanine, 4-Amino-L-phenylalanine, 3-[3,4-bis(trifluoromethyl)phenyl]-L-alanine, Bpa-OH, D-Bpa-OH, 4-tert-butyl-Phe-OH, 4-tert-butyl-D-Phe-OH, 4-(amino)-L-phenylalanine, rac-β²-homophenylalanine, 2-methoxy-L-phenylalanine, (S)-4-methoxy-β-Phe-OH, 2-nitro-L-phenylalanine, pentafluoro-D-phenylalanine, pentafluoro-L-phenylalanine, Phe(4-Br)—OH, D-Phe(4-Br)—OH, Phe(2-CF₃)—OH, D-Phe(2-CF₃)—OH, Phe(3-CF₃)—OH, D-Phe(3-CF₃)—OH, Phe(4-CF₃)—OH, D-Phe(4-CF₃)—OH, Phe(2-Cl)—OH, D-Phe(2-Cl)—OH, Phe(2,4-Cl₂)—OH, D-Phe(2,4-Cl₂)—OH, D-Phe(3-Cl)—OH, Phe(3,4-Cl₂)—OH, Phe(4-Cl)—OH, D-Phe(4-Cl)—OH, Phe(2-CN)—OH, D-Phe(2-CN)—OH, D-Phe(3-CN)—OH, Phe(4-CN)—OH, D-Phe(4-CN)—OH, Phe(2-Me)-OH, D-Phe(2-Me)-OH, Phe(3-Me)-OH, D-Phe(3-Me)-OH, Phe(4-Me)-OH, Phe(4-NH₂)—OH, Phe(4-NO₂)—OH, Phe(2-F)—OH, D-Phe(2-F)—OH, Phe(3-F)—OH, D-Phe(3-F)—OH, Phe(3,4-F₂)—OH, D-Phe(3,4-F₂)—OH, Phe(3,5-F₂)—OH, Phe(4-F)—OH, D-Phe(4-F)—OH, Phe(4-I)—OH, D-3,4,5-trifluorophenylalanine, p-Bromo-DL-phenylalanine, 4-Bromo-L-phenylalanine, β-phenyl-D-phenylalanine, 4-Chloro-L-phenylalanine, DL-2,3-Difluorophenylalanine, DL-3,5-Difluorophenylalanine, 3,4-Dihydroxy-L-phenylalanine, 3-(3,4-Dimethoxyphenyl)-L-alanine, N-[(9H-Fluoren-9-ylmethoxy)carbonyl]-2-methoxy-L-phenylalanine, o-Fluoro-DL-phenylalanine, m-Fluoro-L-phenylalanine, m-Fluoro-DL-phenylalanine, p-Fluoro-L-phenylalanine, p-Fluoro-DL-phenylalanine, 4-Fluoro-D-phenylalanine, 2-fluoro-L-phenylalanine methyl ester, p-fluoro-DL-Phe-OMe, D-3-bromophenylalanine, D-4-bromophenylalanine, L-β-(6-chloro-4-pyridinyl)alanine, D-3,5-difluorophenylalanine, L-3-fluorophenylalanine, L-4-fluorophenylalanine, L-β-(1H-5-indolyl)alanine, 2-nitro-L-phenylalanine, pentafluoro-L-phenylalanine, phe(3-br)-oh, Phe(4-Br)—OH, Phe(2-CF₃)—OH, D-Phe(2-CF₃)—OH, Phe(3-CF₃)—OH, D-Phe(3-CF₃)—OH, Phe(4-CF₃)—OH, D-Phe(4-CF₃)—OH, Phe(2-Cl)—OH, D-Phe(2-Cl)—OH, Phe(2,4-Cl₂)—OH, D-Phe(2,4-Cl₂)—OH, Phe(3,4-Cl₂)—OH, D-Phe(3,4-Cl₂)—OH, Phe(4-Cl)—OH, D-Phe(4-Cl)—OH, Phe(2-CN)—OH, D-Phe(2-CN)—OH, D-Phe(3-CN)—OH, Phe(4-CN)—OH, Phe(2-Me)-OH, Phe(3-Me)-OH, D-Phe(3-Me)-OH, Phe(4-NO₂)—OH, D-Phe(4-NO₂)—OH, D-Phe(2-F)—OH, Phe(3-F)—OH, D-Phe(3-F)—OH, Phe(3,4-F₂)—OH, Phe(3,5-F₂)—OH, D-Phe(4-F)—OH, Phe(4-I)—OH, D-Phe(4-I)—OH, 4-(phosphonomethyl)-Phe-OH, L-4-trifluoromethylphenylalanine, 3,4,5-trifluoro-D-phenylalanine, L-3,4,5-trifluorophenylalanine, 6-Hydroxy-DL-DOPA, 4-(Hydroxymethyl)-D-phenylalanine, N-(3-Indolylacetyl)-L-phenylalanine, p-Iodo-D-phenylalanine, 4-Iodo-L-phenylalanine, α-Methyl-D-phenylalanine, α-Methyl-L-phenylalanine, α-Methyl-DL-phenylalanine, α-Methyl-DL-phenylalanine methyl ester, 4-Nitro-D-phenylalanine, 4-Nitro-L-phenylalanine, 4-Nitro-DL-phenylalanine, (S)-(+) Nitrophenylalanine methyl ester, 2-(Trifluoromethyl)-D-phenylalanine, 2-(Trifluoromethyl)-L-phenylalanine, 3-(Trifluoromethyl)-D-phenylalanine, 3-(Trifluoromethyl)-L-phenylalanine, 4-(Trifluoromethyl)-D-phenylalanine, 3,3′,5-Triiodo-L-thyronine, (R)-4-bromo-β-Phe-OH, N-Acetyl-DL-β-phenylalanine, (S)-4-bromo-β-Phe-OH, (R)-4-chloro-β-Homophe-OH, (S)-4-chloro-β-Homophe-OH, (R)-4-chloro-β-Phe-OH, (S)-4-chloro-β-Phe-OH, (S)-2-cyano-β-Homophe-OH, (R)-4-cyano-β-Homophe-OH, (S)-4-cyano-β-Homophe-OH, (R)-3-cyano-β-Phe-OH, (R)-4-cyano-β-Phe-OH, (S)-4-cyano-β-Phe-OH, (R)-3,4-dimethoxy-β-Phe-OH, (S)-3,4-dimethoxy-β-Phe-OH, (R)-4-fluoro-β-Phe-OH, (S)-4-fluoro-β-Phe-OH, (S)-4-iodo-β-Homophe-OH, (S)-3-cyano-β-Homophe-OH, (S)-3,4-difluoro-β-Homophe-OH, (R)-4-fluoro-β-Homophe-OH, (S)-β2-homophenylalanine, (R)-3-methoxy-β-Phe-OH, (S)-3-methoxy-β-Phe-OH, (R)-4-methoxy-β-Phe-OH, (S)-4-methyl-β-Homophe-OH, (R)-2-methyl-β-Phe-OH, (S)-2-methyl-β-Phe-OH, (R)-3-methyl-β-Phe-OH, (S)-3-methyl-β-Phe-OH, (R)-4-methyl-β-Phe-OH, (S)-4-methyl-β-Phe-OH, β-Phe-OH, D-β-Phe-OH, (S)-2-(trifluoromethyl)-β-Homophe-OH, (S)-2-(trifluoromethyl)-β-Homophe-OH, (S)-3-(trifluoromethyl)-β-Homophe-OH, (R)-4-(trifluoromethyl)-β-Homophe-OH, (S)-2-(trifluoromethyl)-β-Phe-OH, (R)-3-(trifluoromethyl)-β-Phe-OH, (S)-3-(trifluoromethyl)-β-Phe-OH, (R)-4-(trifluoromethyl)-β-Phe-OH, (S)-4-(trifluoromethyl)-β-Phe-OH, β-Homophe-OH, D-β-Homophe-OH, (S)-2-methyl-β-Homophe-OH, (S)-3-methyl-β-Homophe-OH, β-Phe-OH, β-D-Phe-OH, (S)-3-(trifluoromethyl)-β-Homophe-OH, L-β-Homophenylalanine, DL-β-Homophenylalanine, DL-β-Phenylalanine, DL-homophenylalanine methyl ester, D-Homophenylalanine, L-Homophenylalanine, DL-Homophenylalanine, D-Homophenylalanine ethyl ester, (R)-β²-homophenylalanine, L-α-methyl-homophenylalanine [Mhphe], L-α-methylphenylalanine [Mphe], D-α-methylphenylalanine [Dmphe], L-N-methyl-homophenylalanine [Nm phe], L-homophenylalanine [Hphe], L-N-methylphenylalanine [Nmphe], D-N-methylphenylalanine [Dnmphe], N-benzylglycine [Nphe] and D-phenylalanine [Dphe, (dF), f]. Each possibility represents a separate embodiment.

Non limiting examples for proline non-conservative amino acids are: homoproline (hPro), (4-hydroxy)Pro (4HyP), (3-hydroxy)Pro (3HyP), gamma-benzyl-proline, gamma-(2-fluoro-benzyl)-proline, gamma-(3-fluoro-benzyl)-proline, gamma-(4-fluoro-benzyl)-proline, gamma-(2-chloro-benzyl)-proline, gamma-(3-chloro-benzyl)-proline, gamma-(4-chloro-benzyl)-proline, gamma-(2-bromo-benzyl)-proline, gamma-(3-bromo-benzyl)-proline, gamma-(4-bromo-benzyl)-proline, gamma-(2-methyl-benzyl)-proline, gamma-(3-methyl-benzyl)-proline, gamma-(4-methyl-benzyl)-proline, gamma-(2-nitro-benzyl)-proline, gamma-(3-nitro-benzyl)-proline, gamma-(4-nitro-benzyl)-proline, gamma-(l-naphthalenylmethyl)-proline, gamma-(2-naphthalenylmethyl)-proline, gamma-(2,4-dichloro-benzyl)-proline, gamma-(3,4-dichloro-benzyl)-proline, gamma-(3,4-difluoro-benzyl)-proline, gamma-(2-trifluoro-methyl-benzyl)-proline, gamma-(3-trifluoro-methyl-benzyl)-proline, gamma-(4-trifluoro-methyl-benzyl)-proline, gamma-(2-cyano-benzyl)-proline, gamma-(3-cyano-benzyl)-proline, gamma-(4-cyano-benzyl)-proline, gamma-(2-iodo-benzyl)-proline, gamma-(3-iodo-benzyl)-proline, gamma-(4-iodo-benzyl)-proline, gamma-(3-phenyl-allyl-benzyl)-proline, gamma-(3-phenyl-propyl-benzyl)-proline, gamma-(4-tert-butyl-benzyl)-proline, gamma-benzhydryl-proline, gamma-(4-biphenyl-methyl)-proline, gamma-(4-thiazolyl-methyl)-proline, gamma-(3-benzothienyl-methyl)-proline, gamma-(2-thienyl-methyl)-proline, gamma-(3-thienyl-methyl)-proline, gamma-(2-furanyl-methyl)-proline, gamma-(2-pyridinyl-methyl)-proline, gamma-(3-pyridinyl-methyl)-proline, gamma-(4-pyridinyl-methyl)-proline, gamma-allyl-proline, gamma-propynyl-proline, alpha-modified-proline residues, pipecolic acid, azetidine-3-carboxylicacid, L-β-Homoproline, L-β³-homoproline, L-β-Homohydroxyproline, hydroxyproline [Hyp], L-α-methylproline [Mpro], D-α-methylproline [Dmpro], L-N-methylproline [Nmpro], D-N-methylproline [Dnmpro], and D-proline [Dpro, (dP), p]. Each possibility represents a separate embodiment.

Non limiting examples for serine non-conservative amino acids are: (2R,3S)-3-phenylisoserine, D-cycloserine, L-Isoserine, DL-Isoserine, DL-3-Phenylserine, L-β-Homoserine, D-Homoserine, D-Homoserine, L-3-Homoserine, L-homoserine, L-α-methylserine [Mser], D-α-methylserine [Dmser], L-N-methylserine [Nmser], D-N-methylserine [Dnmser], D-serine [Dser, (dS), s], N-(hydroxymethyl)glycine [Nser] and phosphoserine [pSer]. Each possibility represents a separate embodiment.

Non limiting examples for threonine non-conservative amino acids are: L-allo-Threonine, D-Thyroxine, L-β-Homothreonine, L-α-methylthreonine [Mthr], D-α-methylthreonine [Dmthr], L-N-methylthreonine [Nmthr], D-N-methylthreonine [Dnmthr], D-threonine [Dthr, (dT), t], N-(1-hydroxyethyl)glycine [Nthr] and phosphothreonine [pThr]. Each possibility represents a separate embodiment.

Non limiting examples for tryptophan non-conservative amino acids are: 5-Fluoro-L-tryptophan, 5-Fluoro-DL-tryptophan, 5-Hydroxy-L-tryptophan, 5-Methoxy-DL-tryptophan, L-abrine, 5-Methyl-DL-tryptophan, H-Tpi-OMe. β-Homotrp-OMe, L Homotryptophan, L-α-methyltryptophan [Mtrp], D-α-methyltryptophan [Dmtrp], L-N-methyltryptophan [Nmtrp], D-N-methyltryptophan [Dnmtrp], N-(3-indolylethyl)glycine [Nhtrp], D-tryptophan [Dtrp, (dW), w]. Each possibility represents a separate embodiment.

Non limiting examples for tyrosine non-conservative amino acids are: 3,5 diiodotyrosine (3,5-dlTyr), 3,5 diBromotyrosine (3,5-dBTyr), homotyrosine, D-tyrosine, 3-amino-L-tyrosine, 3-amino-D-tyrosine, 3-iodo-L-tyrosine, 3-iodo-D-tyrosine, 3-methoxy-L-tyrosine, 3-methoxy-D-tyrosine, L-thyroxine, D-thyroxine, L-thyronine, D-thyronine, O-methyl-L-tyrosine, O-methyl-D-tyrosine, D-thyronine, O-ethyl-L-tyrosine, O-ethyl-D-tyrosine, 3,5,3′-triiodo-L-thyronine, 3,5,3′-triiodo-D-thyronine, 3,5-diiodo-L-thyronine, 3,5-diiodo-D-thyronine, D-meta-tyrosine, L-meta-tyrosine, D-ortho-tyrosine, L-ortho-tyrosine, phenylalanine, substituted phenylalanine, N-nitro phenylalanine, p-nitro phenylalanine, 3-chloro-Dtyr-oh, Tyr(3,5-dil), 3-Chloro-L-tyrosine, Tyr(3-NO₂)—OH, Tyr(3,5-dil)-OH, N-Me-Tyr-OH, α-Methyl-DL-tyrosine, 3-Nitro-L-tyrosine, DL-o-Tyrosine, β-Homotyr-OH, (R)-3-Tyr-OH, (S)-3-Tyr-OH, L-α-methyltyrosine [Mtyr], D-α-methyltyrosine [Dmtyr], L-N-methyltyrosine [Nmtyr], D-N-methyltyrosine [Dnmtyr], D-tyrosine [Dtyr, (dY), y], O-methyl-tyrosine, and phosphotyrosine [pTyr]. Each possibility represents a separate embodiment.

Non limiting examples for valine non-conservative amino acids are: 3-Fluoro-DL-valine, 4,4,4,4′,4′,4′-Hexafluoro-DL-valine, D-valine [Dval, (dV), v], N-Me-Val-OH [Nmval], N-Me-Val-OH, L-α-methylvaline [Mval], D-α-methylvaline [Dmval], (R)-(+)-α-Methylvaline, (S)-(−)-α-Methylvaline and D-N-methylvaline [Dnmval]. Each possibility represents a separate embodiment.

Other non-natural amino acids that may be substituted as non-conservative replacements include: Ornithine and its modifications: D-Ornithine [Dorn], L-Ornithine [Orn], DL-Ornithine, L-α-methylornithine [Morn], D-α-methylornithine [Dmorn], L-N-methylornithine [Nmorn], D-N-methylornithine [Dnmorn] and N-(3-aminopropyl)glycine [Norn]. Each possibility represents a separate embodiment.

Alicyclic amino acids: L-2,4-Diaminobutyric acid, L-2,3-Diaminopropionic Acid, N-Me-Aib-OH, (R)-2-(amino)-5-hexynoic acid, piperidine-2-carboxylic acid, aminonorbornyl-carboxylate [Norb], alpha-aminobutyric acid [Abu], aminocyclopropane-carboxylate [Cpro], (cis)-3-Aminobicyclo[2.2.1]heptane-2-carboxylic acid, exo-cis-3-Aminobicyclo[2.2.1]hept-5-ene-2-carboxylic acid, 1-Amino-1-cyclobutanecarboxylic acid, cis-2-Aminocycloheptanecarboxylic acid, 1-Aminocyclohexanecarboxylic acid, cis Aminocyclohexanecarboxylic acid, trans-2-Aminocyclohexanecarboxylic acid, cis Amino-3-cyclohexene-1-carboxylic acid, 2-(1-Aminocyclohexyl)acetic acid, cis-2-Amino cyclooctanecarboxylic acid, cis-2-Amino-3-cyclooctene-1-carboxylic acid, (1R,2S)-(−) Amino-1-cyclopentanecarboxylic acid, (1 S,2R)-(+)-2-Amino-1-cyclopentanecarboxylic acid, cis-2-Amino-1-cyclopentanecarboxylic acid, 2-(1-Aminocyclopentyl)acetic acid, cis-2-Amino-2-methylcyclohexanecarboxylic acid, cis-2-Amino methylcyclopentanecarboxylic acid, 3-Amino-3-(4-nitrophenyl)propionic acid, 3-Azetidinecarboxylic acid, amchc-oh, 1-aminocyclobutane carboxylic acid, 1-(amino)cyclohexanecarboxylic acid, cis-2-(amino)-cyclohexanecarboxylic acid, trans-2-(amino)-cyclohexanecarboxylic acid, cis-4-(amino)cyclohexanecarboxylic acid, trans-4-(amino)cyclohexanecarboxylic acid, (±)-cis-2-(amino)-3-cyclohexene-1-carboxylic acid, (±)-cis-6-(amino)-3-cyclohexene-1-carboxylic acid, 2-(1-aminocyclohexyl)acetic acid, cis-[4-(amino)cyclohexyl]acetic acid, 1-(amino)cyclopentanecarboxylic acid, (±)-cis-2-(amino)cyclopentanecarboxylic acid, (1R,4S)-(+)-4-(amino)-2-cyclopentene-1-carboxylic acid, (±)-cis-2-(amino)-3-cyclopentene-1-carboxylic acid, 2-(1-aminocyclopentyl)acetic acid, 1-(amino)cyclopropanecarboxylic acid, Ethyl 1-aminocyclopropanecarboxylate, 1,2-trans-achec-oh, 1-(amino)cyclobutanecarboxylic acid, 1-(amino)cyclohexanecarboxylic acid, cis-2-(amino)-cyclohexanecarboxylic acid, trans-2-(amino)cyclohexanecarboxylic acid, cis-4-(amino)cyclohexanecarboxylic acid, trans-4-(amino)cyclohexanecarboxylic acid, cis-[4-(amino)cyclohexyl]acetic acid, 1-(amino)cyclopentanecarboxylic acid, (1R,4S)-(+)-4-(amino)-2-cyclopentene-1-carboxylic acid, (1 S,4R)-(−)-4-(amino)-2-cyclopentene-1-carboxylic acid, 1-(amino)cyclopropanecarboxylic acid, trans-4-(aminomethyl)cyclohexanecarboxylic acid, β-Dab-OH, 3-Amino-3-(3-bromophenyl)propionic acid, 3-Aminobutanoic acid, cis-2-Amino-3-cyclopentene-1-carboxylic acid, DL-3-Aminoisobutyric acid, (R)-3-Amino-2-phenylpropionic acid, (±)-3-(amino)-4-(4-biphenylyl)butyric acid, cis-3-(amino)cyclohexanecarboxylic acid, (1S,3R)-(+)-3-(amino)cyclopentanecarboxylic acid, (2R,3R)-3-(amino)-2-hydroxy-4-phenylbutyric acid, (2S,3R)-3-(amino)-2-hydroxy-4-phenylbutyric acid, 2-(aminomethyl)phenylacetic acid, (R)-3-(amino)-2-methylpropionic acid, (S)-3-(amino)-2-methylpropionic acid, (R)-3-(amino)-4-(2-naphthyl)butyric acid, (S)-3-(amino)-4-(2-naphthyl)butyric acid, (R)-3-(amino)-5-phenylpentanoic acid, (R)-3-(amino)-2-phenylpropionic acid, Ethyl 3-(benzylamino)propionate, cis-3-(amino)cyclohexanecarboxylic acid, (S)-3-(amino)-5-hexenoic acid, (R)-3-(amino)-2-methylpropionic acid, (S)-3-(amino)-2-methylpropionic acid, (R)-3-(amino)-4-(2-naphthyl)butyric acid, (S)-3-(amino)-4-(2-naphthyl)butyric acid, (R)-(−)-Pyrrolidine-3-carboxylic acid, (S)-(+)-Pyrrolidine-3-carboxylic acid, N-methyl-γ-aminobutyrate [Nmgabu], γ-aminobutyric acid [Gabu], N-methyl-α-amino-α-methylbutyrate [Nmaabu], α-amino-α-methylbutyrate [Aabu], N-methyl-α-aminoisobutyrate [Nmaib], α-aminoisobutyric acid [Aib], α-methyl-y-aminobutyrate [Mgabu]. Each possibility represents a separate embodiment.

Phenyl glycine and its modifications: Phg-OH, D-Phg-OH, 2-(piperazino)-2-(3,4-dimethoxyphenyl)acetic acid, 2-(piperazino)-2-(2-fluorophenyl)acetic acid, 2-(4-piperazino)-2-(3-fluorophenyl)acetic acid, 2-(4-piperazino)-2-(4-methoxyphenyl)acetic acid, 2-(4-piperazino)-2-(3-pyridyl)acetic acid, 2-(4-piperazino)-2-[4-(trifluoromethyl)phenyl]acetic acid, L-(+)-2-Chlorophenylglycine, (±)-2-Chlorophenylglycine, (±)-4-Chlorophenylglycine, (R)-(−)-2-(2,5-Dihydrophenyl)glycine, (R)-(−)-N-(3,5-Dinitrobenzoyl)-α-phenylglycine, (S)-(+)-N-(3,5-Dinitrobenzoyl)-α-phenylglycine, 2,2-Diphenylglycine, 2-Fluoro-DL-α-phenylglycine, 4-Fluoro-D-α-phenylglycine, 4-Hydroxy-D-phenylglycine, 4-Hydroxy-L-phenylglycine, 2-Phenylglycine, D-(−)-α-Phenylglycine, D-(−)-α-Phenylglycine, DL-α-Phenylglycine, L-(+)-α-Phenylglycine, N-Phenylglycine, (R)-(−)-2-Phenylglycine methyl ester, (S)-(+)-2-Phenylglycine methyl ester, 2-Phenylglycinonitrile hydrochloride, α-Phenylglycinonitrile, 3-(Trifluoromethyl)-DL-phenylglycine, and 4-(Trifluoromethyl)-L-phenylglycine. Each possibility represents a separate embodiment.

Penicillamine and its modifications: N-Acetyl-D-penicillamine, D-Penicillamine, L-Penicillamine [Pen], DL-Penicillamine. α-methylpenicillamine [Mpen], N-methylpenicillamine [Nmpen]. Each possibility represents a separate embodiment.

β-Homopyrrolidine. Each possibility represents a separate embodiment.

Aromatic amino acids: 3-Acetamidobenzoic acid, 4-Acetamidobenzoic acid, 4-Acetamido-2-methylbenzoic acid, N-Acetylanthranilic acid, 3-Aminobenzoic acid, 3-Aminobenzoic acid hydrochloride, 4-Aminobenzoic acid, 4-Aminobenzoic acid, 4-Aminobenzoic acid, 4-Aminobenzoic acid, 4-Aminobenzoic acid, 4-Aminobenzoic acid, 2-Aminobenzophenone-2′-carboxylic acid, 2-Amino-4-bromobenzoic acid, 2-Amino-5-bromobenzoic acid, 3-Amino-2-bromobenzoic acid, 3-Amino-4-bromobenzoic acid, 3-Amino-5-bromobenzoic acid, 4-Amino-3-bromobenzoic acid, 5-Amino-2-bromobenzoic acid, 2-Amino-3-bromo-5-methylbenzoic acid, 2-Amino-3-chlorobenzoic acid, 2-Amino-4-chlorobenzoic acid, 2-Amino-5-chlorobenzoic acid, 2-Amino-5-chlorobenzoic acid, 2-Amino-6-chlorobenzoic acid, 3-Amino-2-chlorobenzoic acid, 3-Amino-4-chlorobenzoic acid, 4-Amino-2-chlorobenzoic acid, 4-Amino-3-chlorobenzoic acid, 5-Amino-2-chlorobenzoic acid, 5-Amino-2-chlorobenzoic acid, 4-Amino-5-chloro-2-methoxybenzoic acid, 2-Amino-5-chloro-3-methylbenzoic acid, 3-Amino-2,5-dichlorobenzoic acid, 4-Amino-3,5-dichlorobenzoic acid, 2-Amino-4,5-dimethoxybenzoic acid, 4-(2-Aminoethyl)benzoic acid hydrochloride, 2-Amino-4-fluorobenzoic acid, 2-Amino fluorobenzoic acid, 2-Amino-6-fluorobenzoic acid, 4-Amino-2-fluorobenzoic acid, 2-Amino-5-hydroxybenzoic acid, 3-Amino-4-hydroxybenzoic acid, 4-Amino hydroxybenzoic acid, 2-Amino-5-iodobenzoic acid, 5-Aminoisophthalic acid, 2-Amino methoxybenzoic acid, 2-Amino-4-methoxybenzoic acid, 2-Amino-5-methoxybenzoic acid, 3-Amino-2-methoxybenzoic acid, 3-Amino-4-methoxybenzoic acid, 3-Amino methoxybenzoic acid, 4-Amino-2-methoxybenzoic acid, 4-Amino-3-methoxybenzoic acid, 5-Amino-2-methoxybenzoic acid, 2-Amino-3-methylbenzoic acid, 2-Amino methylbenzoic acid, 2-Amino-6-methylbenzoic acid, 3-(Aminomethyl)benzoic acid, 3-Amino-2-methylbenzoic acid, 3-Amino-4-methylbenzoic acid, 4-(Aminomethyl)benzoic acid, 4-Amino-2-methylbenzoic acid, 4-Amino-3-methylbenzoic acid, 5-Amino-2-methylbenzoic acid, 3-Amino-2-naphthoic acid, 6-Amino-2-naphthoic acid, 2-Amino-3-nitrobenzoic acid, 2-Amino-5-nitrobenzoic acid, 2-Amino-5-nitrobenzoic acid, 4-Amino-3-nitrobenzoic acid, 5-Amino-2-nitrobenzoic acid, 3-(4-Aminophenyl)propionic acid, 3-Aminophthalic acid, 4-Aminophthalic acid, 3-Aminosalicylic acid, 4-Aminosalicylic acid, 5-Aminosalicylic acid, 5-Aminosalicylic acid, 2-Aminoterephthalic acid, 2-Amino-3,4,5,6-tetrafluorobenzoic acid, 4-Amino-2,3,5,6-tetrafluorobenzoic acid, (R)-2-Amino-1,2,3,4-tetrahydronaphthalene-2-carboxylic acid, (S)-2-Amino-1,2,3,4-tetrahydro-2-naphthalenecarboxylic acid, 2-Amino-3-(trifluoromethyl)benzoic acid, 2-Amino-3-(trifluoromethyl)benzoic acid, 3-Amino-5-(trifluoromethyl)benzoic acid, 5-Amino-2,4,6-triiodoisophthalic acid, 2-Amino-3,4,5-trimethoxybenzoic acid, 2-Anilinophenylacetic acid, 2-Abz-OH, 3-Abz-OH, 4-Abz-OH, 2-(aminomethyl)benzoic acid, 3-(aminomethyl)benzoic acid, 4-(aminomethyl)benzoic acid, tert-Butyl 2-aminobenzoate, tert-Butyl 3-aminobenzoate, tert-Butyl 4-aminobenzoate, 4-(Butylamino)benzoic acid, 2,3-Diaminobenzoic acid, 3,4-Diaminobenzoic acid, 3,5-Diaminobenzoic acid, 3,5-Diaminobenzoic acid, 3,5-Dichloroanthranilic acid, 4-(Diethylamino)benzoic acid, 4,5-Difluoroanthranilic acid, 4-(Dimethylamino)benzoic acid, 4-(Dimethylamino)benzoic acid, 3,5-Dimethylanthranilic acid, 5-Fluoro-2-methoxybenzoic acid, 2-Abz-OH, 3-Abz-OH, 4-Abz-OH, 3-(aminomethyl)benzoic acid, 4-(aminomethyl)benzoic acid, 4-(2-hydrazino)benzoic acid, 3-Hydroxyanthranilic acid, 3-Hydroxyanthranilic acid, Methyl 3-aminobenzoate, 3-(Methylamino)benzoic acid, 4-(Methylamino)benzoic acid, Methyl 2-amino-4-chlorobenzoate, Methyl 2-amino-4,5-dimethoxybenzoate, 4-Nitroanthranilic acid, N-Phenylanthranilic acid, N-Phenylanthranilic acid, and Sodium 4-aminosalicylate. Each possibility represents a separate embodiment.

Other amino acids: (S)-α-Amino-γ-butyrolactone, DL-2-Aminocaprylic acid, 7-Aminocephalosporanic acid, 4-Aminocinnamic acid, (S)-(+)-α-Aminocyclohexanepropionic acid, (R)-Amino-(4-hydroxyphenyl)acetic acid methyl ester, 5-Aminolevulinic acid, 4-Amino-nicotinic acid, 3-Aminophenylacetic acid, 4-Aminophenylacetic acid, 2-Amino-2-phenylbutyric acid, 4-(4-Aminophenyl)butyric acid, 2-(4-Aminophenylthio)acetic acid, DL-α-Amino-2-thiopheneacetic acid, 5-Aminovaleric acid, 8-Benzyl (S)-2-aminooctanedioate, 4-(amino)-1-methylpyrrole-2-carboxylic acid, 4-(amino)tetrahydrothiopyran-4-carboxylic acid, (1R,3S,4S)-2-azabicyclo[2.2.1]heptane carboxylic acid, L-azetidine-2-carboxylic acid, azetidine-3-carboxylic acid, 4-(amino)piperidine-4-carboxylic acid, diaminoacetic acid, Inp-OH, (R)-Nip-OH, (S) oxopiperidine-2-carboxylic acid, 2-(4-piperazino)-2-(4-fluorophenyl)acetic acid, 2-(4-piperazino)-2-phenylacetic acid, 4-piperidineacetaldehyde, 4-piperidylacetic acid, (−)-L-thioproline, Tie-OH, 3-piperidinecarboxylic acid, L-(+)-Canavanine, (±)-Carnitine, Chlorambucil, 2,6-Diaminopimelic acid, meso-2,3-Diaminosuccinic acid, 4-(Dimethylamino)cinnamic acid, 4-(Dimethylamino)phenylacetic acid, Ethyl (S)—N-Boc-piperidine-3-carboxylate, Ethyl piperazinoacetate, 4-[2-(amino)ethyl]piperazin-1-ylacetic acid, (R)-4-(amino)-5-phenylpentanoic acid, (S)-azetidine-2-carboxylic acid, azetidine-3-carboxylic acid, guvacine, Inp-OH, (R)-Nip-OH, DL-Nip-OH, 4-phenyl-piperidine-4-carboxylic acid, 1-piperazineacetic acid, 4-piperidineacetic acid, (R)-piperidine-2-carboxylic acid, (S)-piperidine-2-carboxylic acid, (S)-1,2,3,4-tetrahydronorharmane-3-carboxylic acid, Tic-OH, D-Tic-OH, Iminodiacetic acid, Indoline-2-carboxylic acid, DL-Kynurenine, L-aziridine-2-carboxylate, Methyl 4-aminobutyrate, (S)-2-Piperazinecarboxylic acid, 2-(1-Piperazinyl)acetic acid, (R)-(−)-3-Piperidinecarboxylic acid, 2-Pyrrolidone-5-carboxylic acid, (R)-(+)-2-Pyrrolidone-5-carboxylic acid, (R)-1,2,3,4-Tetrahydro-3-isoquinolinecarboxylic acid, (S)-1,2,3,4-Tetrahydro-3-isoquinolinecarboxylic acid, L-4-Thiazolidinecarboxylic acid, (4R)-(−)-2-Thioxo-4-thiazolidinecarboxylic acid, hydrazinoacetic acid, and 3,3′,5-Triiodo-L-thyronine. Each possibility represents a separate embodiment.

The present disclosure provides peptides comprising peptidomimetic compounds having further improved stability and cell permeability properties. Some embodiments comprise a peptide according to any of SEQ ID NO: 1-64 and 69-79, wherein one of more peptide bonds (—CO—NH—) within the peptide may be substituted, for example, by N-methylated amide bonds (—N(CH₃)—CO—), ester bonds (—C(═O)—O—), ketomethylene bonds (—CO—CH₂—), sulfinylmethylene bonds (—S(═O)—CH₂—), α-aza bonds (—NH—N(R)—CO—), wherein R is any alkyl (e.g., methyl), amine bonds (—CH₂—NH—), sulfide bonds (—CH₂—S—), ethylene bonds (—CH₂CH₂—), hydroxyethylene bonds (—CH(OH)—CH₂—), thioamide bonds (—CS—NH—), olefinic double bonds (—CH═CH—), fluorinated olefinic double bonds (—CF═CH—), or retro amide bonds (—NH—CO—), peptide derivatives (—N(R^{x})—CH₂—CO—), wherein R^{x} is the “normal” side chain, naturally present on the carbon atom. These modifications can occur at any of the bonds along the peptide chain and even at several (2-3) bonds at the same time.

Size variants of the peptides described herein are specifically contemplated. Exemplary peptides are composed of 6 to 50 amino acids. All integer subranges of 6-50 amino acids (e.g., 7-50 aa, 8-50 aa, 9-50 aa, 6-49 aa, 6-48 aa, 7-49 aa, and so on) are specifically contemplated as genera of the invention; and all integer values are contemplated as species of the invention. In exemplary embodiments, the peptide comprises at least seven or eight amino acids connected via peptide bonds. In exemplary aspects, the peptide is at least about 9 amino acids in length, at least about 10 amino acids in length, at least about 11 amino acids in length, at least about 12 amino acids in length, or at least about 13 amino acids in length. In exemplary aspects, the peptide is at least about 14 amino acids in length, at least about 15 amino acids in length, at least about 16 amino acids in length, or at least about 17 amino acids in length. In exemplary aspects, the peptide is at least about 18 amino acids in length, at least about 19 amino acids in length, or at least about 20, 21, 22, 23, 24, 25, 26, 27, 28, 29, or 30 amino acids in length. In exemplary aspects, the peptide is less than about 50 amino acids in length, less than about 40 amino acids, or less than about 30 amino acids, or less than about 25 amino acids in length. In exemplary aspects, the peptide is about 8 to about 30 amino acids in length or about 8 to about 20 amino acids in length. In exemplary aspects, the peptide is about 10 to about 10 amino acids in length, about 14 to about 20 amino acids in length. In exemplary aspects, the peptide is 8-9, 10-11, 12-13, 14-15, or 16-17 amino acids in length. In some embodiments, the peptide is a 8 mer, 9-mer, 10-mer, 11-mer, 12-mer, 13-mer, 14-mer, 15-mer, 16-mer, 17-mer, 18-mer, 19-mer, or 20-mer.

The peptides of some embodiments are preferably utilized in a linear form, although it will be appreciated that in cases where cyclization does not severely interfere with peptide characteristics, cyclic forms of the peptide can also be utilized and are contemplated as embodiments.

According to some embodiments, conjugates comprising any of the peptides and analogs described herein conjugated to a moiety for extending half-life or increasing cell penetration. For example, the half-life extending moiety may be a peptide or protein and the conjugate is a fusion protein or chimeric polypeptide. Alternatively, the half-life extending moiety may be a polymer, e.g., a polyethylene glycol. The present disclosures furthermore provide dimers and multimers comprising any of the peptides and analogs described herein.

Any moiety known in the art to facilitate actively or passively or enhance permeability of the peptides into cells may be used for conjugation with the peptide core. Non-limitative examples include: hydrophobic moieties such as fatty acids, steroids and bulky aromatic or aliphatic compounds; moieties which may have cell-membrane receptors or carriers, such as steroids, vitamins and sugars, natural and non-natural amino acids and transporter peptides. According to a preferred embodiment, the hydrophobic moiety is a lipid moiety or an amino acid moiety. The permeability-enhancing moiety may be connected to any position in the peptide moiety, directly or through a spacer or linker, preferably to the amino terminus of the peptide moiety. The hydrophobic moiety may preferably comprise a lipid moiety or an amino acid moiety. According to a specific embodiment the hydrophobic moiety is selected from the group consisting of: phospholipids, steroids, sphingosines, ceramides, octyl-glycine, 2-cyclohexylalanine, benzolylphenylalanine, propionoyl (C₃); butanoyl (C₄); pentanoyl (C₅); caproyl (C₆); heptanoyl (C₇); capryloyl (C₈); nonanoyl (C₉); capryl (C₁₀); undecanoyl (C₁₁); lauroyl (C₁₂); tridecanoyl (C₁₃); myristoyl (C₁₄); pentadecanoyl (C₁₅); palmitoyl (C₁₆); phtanoyl ((CH₃)₄); heptadecanoyl (C₁₆); stearoyl (C₁₈); nonadecanoyl (C₁₉); arachidoyl (C₂₀); heniecosanoyl (C₂₁); behenoyl (C₂₂); trucisanoyl (C₂₃); and lignoceroyl (C₂₄); wherein said hydrophobic moiety is attached to said chimeric polypeptide with amide bonds, sulfhydryls, amines, alcohols, phenolic groups, or carbon-carbon bonds. Other examples of lipidic moieties which may be used include: Lipofectamine, Transfectace, Transfectam, Cytofectin, DMRIE, DLRIE, GAP-DLRIE, DOTAP, DOPE, DMEAP, DODMP, DOPC, DDAB, DOSPA, EDLPC, EDMPC, DPH, TMADPH, CTAB, lysyl-PE, DC-Cho, -alanyl cholesterol; DOGS, DPPES, DOPE, DMAP, DMPE, DOGS, DOHME, DPEPC, Pluronic, Tween, BRIJ, plasmalogen, phosphatidylethanolamine, phosphatidylcholine, glycerol-3-ethylphosphatidylcholine, dimethyl ammonium propane, trimethyl ammonium propane, diethylammonium propane, triethylammonium propane, dimethyldioctadecylammonium bromide, a sphingolipid, sphingomyelin, a lysolipid, a glycolipid, a sulfatide, a glycosphingolipid, cholesterol, cholesterol ester, cholesterol salt, oil, N-succinyldioleoylphosphatidylethanolamine, 1,2-dioleoyl-glycerol, 1,3-dipalmitoyl-2-succinylglycerol, 1,2-dipalmitoyl-3-succinylglycerol, 1-hexadecyl-2-palmitoylglycerophosphatidylethanolamine, palmitoylhomocystiene, N,N′-Bis (dodecyaminocarbonylmethylene)-N,N′-bis((-N,N,N-trimethylammoniumethyl-aminocarbonylmethylene)ethylenediamine tetraiodide; N,N″-Bis(hexadecylaminocarbonylmethylene)-N,N′,N″-tris((-N,N,N-trimethylammonium-ethylaminocarbonylmethylenediethylenetriamine hexaiodide; N,N′-Bis(dodecylaminocarbonylmethylene)-N,N″-bis((-N,N,N-trimethylammonium ethylaminocarbonylmethylene)cyclohexylene-1,4-diamine tetraiodide; 1,7,7-tetra-((N,N,N,N-tetramethylammoniumethylamino-carbonylmethylene) hexadecylarninocarbonyl-methylene-1,3,7-triaazaheptane heptaiodide; N,N,N′,N′-tetra((N,N,N-trimethylammonium-ethylaminocarbonylmethylene)-N′-(1,2-dioleoylglycero-3-phosphoethanolamino-carbonylmethylene)diethylenetriamine tetraiodide; dioleoylphosphatidylethanolamine, a fatty acid, a lysolipid, phosphatidylcholine, phosphatidylethanolamine, phosphatidylserine, phosphatidylglycerol, phosphatidylinositol, a sphingolipid, a glycolipid, a glucolipid, a sulfatide, a glycosphingolipid, phosphatidic acid, palmitic acid, stearic acid, arachidonic acid, oleic acid, a lipid bearing a polymer, a lipid bearing a sulfonated saccharide, cholesterol, tocopherol hemisuccinate, a lipid with an ether-linked fatty acid, a lipid with an ester-linked fatty acid, a polymerized lipid, diacetyl phosphate, stearylamine, cardiolipin, a phospholipid with a fatty acid of 6-8 carbons in length, a phospholipid with asymmetric acyl chains, 6-(5-cholesten-3b-yloxy)-I-thio-b-D-galactopyranoside, digalactosyldiglyceride, 6-(5-cholesten-3b-yloxy)hexyl-6-amino-6-deoxy-1-thio-b-D-galactopyranoside, 6-(5-cholesten-3b-yloxy)hexyl-6-amino-6-deoxyl-1-thio-α-D-mannopyranoside, 12-(((7′-diethylamino-coumarin-3-yl)carbonyl)methylamino)-octadecanoic acid; N-[12-(((7′-diethylaminocoumarin-3-yl)carbonyl)methyl-amino) octadecanoyl]-2-aminopalmitic acid; cholesteryl)4′-trimethyl-ammonio)butanoate; N-succinyldioleoyl-phosphatidylethanolamine; 1,2-dioleoyl-glycerol; 1,2-dipalmitoyl-3-succinyl-glycerol; 1,3-dipalmitoyl-2-succinylglycerol, l-hexadecyl-2-palmitoylglycero-phosphoethanolamine, and palmitoylhomocysteine.

The peptides disclosed herein may be conjugated to one or more moieties that cause the conjugate to function as a prodrug. For example, the N-amino acid related moieties described in U.S. Pat. No. 8,969,288 and US Patent Application Pub. 20160058881, both incorporated herein by reference in their entirety, can be conjugated to the peptides disclosed herein and such conjugates are included in this disclosure.

According to some embodiments the peptides may be attached (either covalently or non-covalently) to a penetrating agent. As used herein the phrase “penetrating agent” refers to an agent which enhances translocation of any of the attached peptide across a cell membrane. Typically, peptide based penetrating agents have an amino acid composition containing either a high relative abundance of positively charged amino acids such as lysine or arginine, or have sequences that contain an alternating pattern of polar/charged amino acids and non-polar, hydrophobic amino acids. By way of a non-limiting example, cell penetrating peptide (CPP) sequences may be used in order to enhance intracellular penetration. CPPs may include short and long versions of the protein transduction domain (PTD) of HIV TAT protein, such as for example, YARAAARQARA (SEQ ID NO: 65), YGRKKRR (SEQ ID NO: 66), YGRKKRRQRRR (SEQ ID NO: 67), or RRQRR (SEQ ID NO: 68)]. However, the disclosure is not so limited, and any suitable penetrating agent may be used, as known by those of skill in the art. Another method of enhancing cell penetration is via N-terminal myristoylation. In this protein modification, a myristoyl group (derived from myristic acid) is covalently attached via an amide bond to the alpha-amino group of an N-terminal amino acid of the peptide.

According to some embodiments the peptide is modified to include a duration enhancing moiety. The duration enhancing moiety can be a water soluble polymer, or a long chain aliphatic group. In some embodiments, a plurality of duration enhancing moieties may be attached to the peptide, in which case each linker to each duration enhancing moiety is independently selected from the linkers described herein.

According to some embodiments the amino terminus of the peptide is modified, e.g. acylated. According to additional embodiments the carboxy terminus is modified, e.g., it may be acylated, amidated, reduced or esterified. In accordance with some embodiments, the peptide comprises an acylated amino acid (e.g., a non-coded acylated amino acid (e.g., an amino acid comprising an acyl group which is non-native to a naturally-occurring amino acid)). In accordance with one embodiment, the peptide comprises an acyl group which is attached to the peptide via an ester, thioester, or amide linkage for purposes of prolonging half-life in circulation and/or delaying the onset of and/or extending the duration of action and/or improving resistance to proteases. Acylation can be carried out at any position within the peptide, (e.g., the amino acid at the C-terminus), provided that activity is retained, if not enhanced. The peptide in some embodiments can be acylated at the same amino acid position where a hydrophilic moiety is linked, or at a different amino acid position. The acyl group can be covalently linked directly to an amino acid of the peptide, or indirectly to an amino acid of the peptide via a spacer, wherein the spacer is positioned between the amino acid of the peptide and the acyl group.

In specific aspects, the peptide is modified to comprise an acyl group by direct acylation of an amine, hydroxyl, or thiol of a side chain of an amino acid of the peptide. In this regard, the acylated peptide can comprise the amino acid sequence of any of SEQ ID NO: 1-64 and 69-79, or a modified amino acid sequence thereof comprising one or more of the amino acid modifications described herein.

In some embodiments, the peptide comprises a spacer between the analog and the acyl group. In some embodiments, the peptide is covalently bound to the spacer, which is covalently bound to the acyl group. In some embodiments, the spacer is an amino acid comprising a side chain amine, hydroxyl, or thiol, or a dipeptide or tripeptide comprising an amino acid comprising a side chain amine, hydroxyl, or thiol. The amino acid to which the spacer is attached can be any amino acid (e.g., a singly or doubly α-substituted amino acid) comprising a moiety which permits linkage to the spacer. For example, an amino acid comprising a side chain NH₂, —OH, or —COOH (e.g., Lys, Orn, Ser, Asp, or Glu) is suitable. In some embodiments, the spacer is an amino acid comprising a side chain amine, hydroxyl, or thiol, or a dipeptide or tripeptide comprising an amino acid comprising a side chain amine, hydroxyl, or thiol. When acylation occurs through an amine group of a spacer, the acylation can occur through the alpha amine of the amino acid or a side chain amine. In the instance in which the alpha amine is acylated, the amino acid of the spacer can be any amino acid. For example, the amino acid of the spacer can be a hydrophobic amino acid, e.g., Gly, Ala, Val, Leu, Ile, Trp, Met, Phe, Tyr, 6-amino hexanoic acid, 5-aminovaleric acid, 7-aminoheptanoic acid, and 8-aminooctanoic acid. Alternatively, the amino acid of the spacer can be an acidic residue, e.g., Asp, Glu, homoglutamic acid, homocysteic acid, cysteic acid, gamma-glutamic acid. In the instance in which the side chain amine of the amino acid of the spacer is acylated, the amino acid of the spacer is an amino acid comprising a side chain amine. In this instance, it is possible for both the alpha amine and the side chain amine of the amino acid of the spacer to be acylated, such that the peptide is diacylated. Embodiments include such diacylated molecules. When acylation occurs through a hydroxyl group of a spacer, the amino acid or one of the amino acids of the dipeptide or tripeptide can be Ser. When acylation occurs through a thiol group of a spacer, the amino acid or one of the amino acids of the dipeptide or tripeptide can be Cys. In some embodiments, the spacer is a hydrophilic bifunctional spacer. In certain embodiments, the hydrophilic bifunctional spacer comprises two or more reactive groups, e.g., an amine, a hydroxyl, a thiol, and a carboxyl group or any combinations thereof. In certain embodiments, the hydrophilic bifunctional spacer comprises a hydroxyl group and a carboxylate. In other embodiments, the hydrophilic bifunctional spacer comprises an amine group and a carboxylate. In other embodiments, the hydrophilic bifunctional spacer comprises a thiol group and a carboxylate.

In a specific embodiment, the spacer comprises an amino poly(alkyloxy)carboxylate. In this regard, the spacer can comprise, for example, NH₂(CH₂CH₂O)ₙ(CH₂)ₘCOOH, wherein m is any integer from 1 to 6 and n is any integer from 2 to 12, such as, e.g., 8-amino-3,6-dioxaoctanoic acid, which is commercially available from Peptides International, Inc. (Louisville, Ky.). In some embodiments, the spacer is a hydrophobic bifunctional spacer. Hydrophobic bifunctional spacers are known in the art. See, e.g., Bioconjugate Techniques, G. T. Hermanson (Academic Press, San Diego, Calif., 1996), which is incorporated by reference in its entirety. In certain embodiments, the hydrophobic bifunctional spacer comprises two or more reactive groups, e.g., an amine, a hydroxyl, a thiol, and a carboxyl group or any combinations thereof. In certain embodiments, the hydrophobic bifunctional spacer comprises a hydroxyl group and a carboxylate. In other embodiments, the hydrophobic bifunctional spacer comprises an amine group and a carboxylate. In other embodiments, the hydrophobic bifunctional spacer comprises a thiol group and a carboxylate. Suitable hydrophobic bifunctional spacers comprising a carboxylate and a hydroxyl group or a thiol group are known in the art and include, for example, 8-hydroxyoctanoic acid and 8-mercaptooctanoic acid. In some embodiments, the bifunctional spacer is not a dicarboxylic acid comprising an unbranched, methylene of 1-7 carbon atoms between the carboxylate groups. In some embodiments, the bifunctional spacer is a dicarboxylic acid comprising an unbranched, methylene of 1-7 carbon atoms between the carboxylate groups. The spacer (e.g., amino acid, dipeptide, tripeptide, hydrophilic bifunctional spacer, or hydrophobic bifunctional spacer) in specific embodiments is 3 to 10 atoms (e.g., 6 to 10 atoms, (e.g., 6, 7, 8, 9, or 10 atoms) in length. In more specific embodiments, the spacer is about 3 to 10 atoms (e.g., 6 to 10 atoms) in length and the acyl group is a C₁₂ to C₁₈ fatty acyl group, e.g., C₁₄ fatty acyl group, C₁₆ fatty acyl group, such that the total length of the spacer and acyl group is 14 to 28 atoms, e.g., about 14, 15, 16, 17, 18, 19, 20, 21, 22, 23, 24, 25, 26, 27, or 28 atoms. In some embodiments, the length of the spacer and acyl group is 17 to 28 (e.g., 19 to 26, 19 to 21) atoms. In accordance with certain foregoing embodiments, the bifunctional spacer can be a synthetic or naturally occurring amino acid (including, but not limited to, any of those described herein) comprising an amino acid backbone that is 3 to 10 atoms in length (e.g., 6-amino hexanoic acid, 5-aminovaleric acid, 7-aminoheptanoic acid, and 8-aminooctanoic acid). Alternatively, the spacer can be a dipeptide or tripeptide spacer having a peptide backbone that is 3 to 10 atoms (e.g., 6 to 10 atoms) in length. Each amino acid of the dipeptide or tripeptide spacer can be the same as or different from the other amino acid(s) of the dipeptide or tripeptide and can be independently selected from the group consisting of: naturally-occurring or coded and/or non-coded or non-naturally occurring amino acids, including, for example, any of the D or L isomers of the naturally-occurring amino acids (Ala, Cys, Asp, Glu, Phe, Gly, His, Ile, Lys, Leu, Met, Asn, Pro, Arg, Ser, Thr, Val, Trp, Tyr), or any D or L isomers of the non-naturally occurring or non-coded amino acids selected from the group consisting of: β-alanine (β-Ala), N-α-methylalanine (Me-Ala), aminobutyric acid (Abu), γ-aminobutyric acid (7-Abu), aminohexanoic acid (c-Ahx), aminoisobutyric acid (Aib), aminomethylpyrrole carboxylic acid, am inopiperidinecarboxylic acid, aminoserine (Ams), aminotetrahydropyran-4-carboxylic acid, arginine N-methoxy-N-methyl amide, β-aspartic acid (β-Asp), azetidine carboxylic acid, 3-(2-benzothiazolyl)alanine, α-tert-butylglycine, 2-amino-5-ureido-n-valeric acid (citrulline, Cit), β-Cyclohexylalanine (Cha), acetamidomethyl-cysteine, diaminobutanoic acid (Dab), diaminopropionic acid (Dpr), dihydroxyphenylalanine (DOPA), dimethylthiazolidine (DMTA), γ-Glutamic acid (γ-Glu), homoserine (Hse), hydroxyproline (Hyp), isoleucine N-methoxy-N-methyl amide, methyl-isoleucine (Melle), isonipecotic acid (Isn), methyl-leucine (MeLeu), methyl-lysine, dimethyl-lysine, trimethyl-lysine, methanoproline, methionine-sulfoxide (Met(0)), methionine-sulfone (Met(02)), norleucine (Nle), methyl-norleucine (Me-Nle), norvaline (Nva), ornithine (Orn), para-aminobenzoic acid (PABA), penicillamine (Pen), methylphenylalanine (MePhe), 4-Chlorophenylalanine (Phe(4-CI)), 4-fluorophenylalanine (Phe(4-F)), 4-nitrophenylalanine (Phe(4-NO₂)), 4-cyanophenylalanine ((Phe(4-CN)), phenylglycine (Phg), piperidinylalanine, piperidinylglycine, 3,4-dehydroproline, pyrrolidinylalanine, sarcosine (Sar), selenocysteine (Sec), O-Benzyl-phosphoserine, 4-amino-3-hydroxy-6-methylheptanoic acid (Sta), 4-amino-5-cyclohexyl-3-hydroxypentanoic acid (ACHPA), 4-amino-3-hydroxy-5-phenylpentanoic acid (AHPPA), 1,2,3,4,-tetrahydro-isoquinoline-3-carboxylic acid (Tic), tetrahydropyranglycine, thienylalanine (Thi), O-benzyl-phosphotyrosine, 0-Phosphotyrosine, methoxytyrosine, ethoxytyrosine, O-(bis-dimethylamino-phosphono)-tyrosine, tyrosine sulfate tetrabutylamine, methyl-valine (MeVal), and alkylated 3-mercaptopropionic acid. In some embodiments, the spacer comprises an overall negative charge, e.g., comprises one or two negative-charged amino acids. In some embodiments, the dipeptide is not any of the dipeptides of general structure A-B, wherein A is selected from the group consisting of Gly, Gln, Ala, Arg, Asp, Asn, Ile, Leu, Val, Phe, and Pro, wherein B is selected from the group consisting of Lys, His, Trp. In some embodiments, the dipeptide spacer is selected from the group consisting of: Ala-Ala, β-Ala-β-Ala, Leu-Leu, Pro-Pro, γ-aminobutyric acid-γ-aminobutyric acid, Glu-Glu, and γ-Glu-γ-Glu.

Suitable methods of peptide acylation via amines, hydroxyls, and thiols are known in the art. See, for example, Miller, Biochem Biophys Res Commun 218: 377-382 (1996); Shimohigashi and Stammer, Int J Pept Protein Res 19: 54-62 (1982); and Previero et al., Biochim Biophys Acta 263: 7-13 (1972) (for methods of acylating through a hydroxyl); and San and Silvius, J Pept Res 66: 169-180 (2005) (for methods of acylating through a thiol); Bioconjugate Chem. “Chemical Modifications of Proteins: History and Applications” pages 1, 2-12 (1990); Hashimoto et al., Pharmaceutical Res. “Synthesis of Palmitoyl Derivatives of Insulin and their Biological Activity” Vol. 6, No: 2 pp. 171-176 (1989). The acyl group of the acylated amino acid can be of any size, e.g., any length carbon chain, and can be linear or branched. In some specific embodiments, the acyl group is a C₄ to C₃₀ fatty acid. For example, the acyl group can be any of a C₄ fatty acid, C₆ fatty acid, C₈ fatty acid, C₁₀ fatty acid, C₁₂ fatty acid, C₁₄ fatty acid, C₁₆ fatty acid, C₁₈ fatty acid, C₂₀ fatty acid, C₂₂ fatty acid, C₂₄ fatty acid, C₂₆ fatty acid, C₂₈ fatty acid, or a C₃₀ fatty acid. In some embodiments, the acyl group is a C₈ to C₂₀ fatty acid, e.g., a C₁₄ fatty acid or a C₁₆ fatty acid. In an alternative embodiment, the acyl group is a bile acid. The bile acid can be any suitable bile acid, including, but not limited to, cholic acid, chenodeoxycholic acid, deoxycholic acid, lithocholic acid, taurocholic acid, glycocholic acid, and cholesterol acid. In some embodiments, the peptide comprises an acylated amino acid by acylation of a long chain alkane on the peptide. In specific aspects, the long chain alkane comprises an amine, hydroxyl, or thiol group (e.g., octadecylamine, tetradecanol, and hexadecanethiol) which reacts with a carboxyl group, or activated form thereof, of the peptide. The carboxyl group, or activated form thereof, of the peptide can be part of a side chain of an amino acid (e.g., glutamic acid, aspartic acid) of the peptide or can be part of the analog backbone. In certain embodiments, the peptide is modified to comprise an acyl group by acylation of the long chain alkane by a spacer which is attached to the peptide. In specific aspects, the long chain alkane comprises an amine, hydroxyl, or thiol group which reacts with a carboxyl group, or activated form thereof, of the spacer. Suitable spacers comprising a carboxyl group, or activated form thereof, are described herein and include, for example, bifunctional spacers, e.g., amino acids, dipeptides, tripeptides, hydrophilic bifunctional spacers and hydrophobic bifunctional spacers.

As used herein, the term “activated form” of a carboxyl group refers to a carboxyl group with the general formula R(C═O)X, wherein X is a leaving group and R is the peptide or the spacer. For example, activated forms of a carboxyl groups may include, but are not limited to, acyl chlorides, anhydrides, and esters. In some embodiments, the activated carboxyl group is an ester with a N-hydroxysuccinimide ester (NHS) leaving group.

With regard to these aspects, in which a long chain alkane is acylated by the peptide or the spacer, the long chain alkane may be of any size and can comprise any length of carbon chain. The long chain alkane can be linear or branched. In certain aspects, the long chain alkane is a C₄ to C₃₀ alkane. For example, the long chain alkane can be any of a C₄ alkane, C₆ alkane, C₈ alkane, C₁₀ alkane, C₁₂ alkane, C₁₄ alkane, C₁₆ alkane, C₁₈ alkane, C₂₀ alkane, C₂₂ alkane, C₂₄ alkane, C₂₆ alkane, 028 alkane, or a C₃₀ alkane. In some embodiments, the long chain alkane comprises a C₈ to C₂₀ alkane, e.g., a C₁₄ alkane, C₁₆ alkane, or a C₁₈ alkane.

Also, in some embodiments, an amine, hydroxyl, or thiol group of the peptide is acylated with a cholesterol acid. In a specific embodiment, the peptide is linked to the cholesterol acid through an alkylated des-amino Cys spacer, i.e., an alkylated 3-mercaptopropionic acid spacer. The alkylated des-amino Cys spacer can be, for example, a des-amino-Cys spacer comprising a dodecaethylene glycol moiety.

The peptides described herein can be further modified to comprise a hydrophilic moiety. In some specific embodiments the hydrophilic moiety can comprise a polyethylene glycol (PEG) chain. The incorporation of a hydrophilic moiety can be accomplished through any suitable means, such as any of the methods described herein. In this regard, the acylated peptide can of any of SEQ ID NOs: 1-64 and 69-79, including any of the modifications described herein, in which at least one of the amino acids comprises an acyl group and at least one of the amino acids is covalently bonded to a hydrophilic moiety (e.g., PEG). In some embodiments, the acyl group is attached via a spacer comprising Cys, Lys, Orn, homo-Cys, or Ac-Phe, and the hydrophilic moiety is incorporated at a Cys residue.

Alternatively, the peptides can comprise a spacer, wherein the spacer is both acylated and modified to comprise the hydrophilic moiety. Nonlimiting examples of suitable spacers include a spacer comprising one or more amino acids selected from the group consisting of Cys, Lys, Orn, homo-Cys, and Ac-Phe.

In accordance with some embodiments, the peptide comprises an alkylated amino acid (e.g., a non-coded alkylated amino acid (e.g., an amino acid comprising an alkyl group which is non-native to a naturally-occurring amino acid)). Alkylation can be carried out at any positions within the peptides, including any of the positions described herein as a site for acylation, including but not limited to, any of amino acid positions, at a position within a C-terminal extension, or at the C-terminus, provided that the biological activity is retained. The alkyl group can be covalently linked directly to an amino acid of the peptides, or indirectly to an amino acid of the peptides via a spacer, wherein the spacer is positioned between the amino acid of the peptides and the alkyl group. The peptides may be alkylated at the same amino acid position where a hydrophilic moiety is linked, or at a different amino acid position. In specific aspects, the peptides may be modified to comprise an alkyl group by direct alkylation of an amine, hydroxyl, or thiol of a side chain of an amino acid of the peptides. In this regard, the alkylated peptides can comprise an amino acid sequence with at least one of the amino acids modified to any amino acid comprising a side chain amine, hydroxyl, or thiol. In yet other embodiments, the amino acid comprising a side chain amine, hydroxyl, or thiol is a disubstituted amino acid. In some embodiments, the alkylated peptide comprises a spacer between the peptide and the alkyl group. In some embodiments, the peptide is covalently bound to the spacer, which is covalently bound to the alkyl group. In some exemplary embodiments, the peptide is modified to comprise an alkyl group by alkylation of an amine, hydroxyl, or thiol of a spacer, which spacer is attached to a side chain of an amino acid. The amino acid to which the spacer is attached can be any amino acid comprising a moiety which permits linkage to the spacer. For example, an amino acid comprising a side chain NH₂, —OH, or —COOH (e.g., Lys, Orn, Ser, Asp, or Glu) is suitable. In some embodiments, the spacer is an amino acid comprising a side chain amine, hydroxyl, or thiol or a dipeptide or tripeptide comprising an amino acid comprising a side chain amine, hydroxyl, or thiol. When alkylation occurs through an amine group of a spacer, the alkylation can occur through the alpha amine of an amino acid or a side chain amine. In the instance in which the alpha amine is alkylated, the amino acid of the spacer can be any amino acid. For example, the amino acid of the spacer can be a hydrophobic amino acid, e.g., Gly, Ala, Val, Leu, Ile, Trp, Met, Phe, Tyr, 6-amino hexanoic acid, 5-aminovaleric acid, 7-aminoheptanoic acid, and 8-aminooctanoic acid. Alternatively, the amino acid of the spacer can be an acidic residue, e.g., Asp and Glu, provided that the alkylation occurs on the alpha amine of the acidic residue. In the instance in which the side chain amine of the amino acid of the spacer is alkylated, the amino acid of the spacer is an amino acid comprising a side chain amine, e.g., an amino acid of Formulas I-IV and II′-III′ (e.g., Lys or Orn). In this instance, it is possible for both the alpha amine and the side chain amine of the amino acid of the spacer to be alkylated, such that the peptide is dialkylated. Embodiments include such dialkylated molecules. When alkylation occurs through a hydroxyl group of a spacer, the amino acid can be Ser. When alkylation occurs through a thiol group of spacer, the amino acid can be Cys. In some embodiments, the spacer is a hydrophilic bifunctional spacer. In certain embodiments, the hydrophilic bifunctional spacer comprises two or more reactive groups, e.g., an amine, a hydroxyl, a thiol, and a carboxyl group or any combinations thereof. In certain embodiments, the hydrophilic bifunctional spacer comprises a hydroxyl group and a carboxylate. In other embodiments, the hydrophilic bifunctional spacer comprises an amine group and a carboxylate. In other embodiments, the hydrophilic bifunctional spacer comprises a thiol group and a carboxylate. In a specific embodiment, the spacer comprises an amino poly(alkyloxy)carboxylate. In this regard, the spacer can comprise, for example, NH₂(CH₂CH₂O)ₙ(CH₂)ₘCOOH, wherein m is any integer from 1 to 6 and n is any integer from 2 to 12, such as, e.g., 8-amino-3,6-dioxaoctanoic acid, which is commercially available from Peptides International, Inc. (Louisville, Ky.). Suitable hydrophobic bifunctional spacers comprising a carboxylate and a hydroxyl group or a thiol group are known in the art and include, for example, 8-hydroxyoctanoic acid and 8-mercaptooctanoic acid. The spacer (e.g., amino acid, dipeptide, tripeptide, hydrophilic bifunctional spacer, or hydrophobic bifunctional spacer) in specific embodiments is 3 to 10 atoms (e.g., 6 to 10 atoms, (e.g., 6, 7, 8, 9, or 10 atoms)) in length. In more specific embodiments, the spacer is about 3 to 10 atoms (e.g., 6 to 10 atoms) in length and the alkyl is a C₁₂ to C₁₈ alkyl group, e.g., C₁₄ alkyl group, C₁₆ alkyl group, such that the total length of the spacer and alkyl group is 14 to 28 atoms, e.g., about 14, 15, 16, 17, 18, 19, 20, 21, 22, 23, 24, 25, 26, 27, or 28 atoms. In some embodiments, the length of the spacer and alkyl is 17 to 28 (e.g., 19 to 26, 19 to 21) atoms. In accordance with certain foregoing embodiments, the bifunctional spacer can be a synthetic or non-naturally occurring or non-coded amino acid comprising an amino acid backbone that is 3 to 10 atoms in length (e.g., 6-amino hexanoic acid, 5-aminovaleric acid, 7-aminoheptanoic acid, and 8-aminooctanoic acid). Alternatively, the spacer can be a dipeptide or tripeptide spacer having a peptide backbone that is 3 to 10 atoms (e.g., 6 to 10 atoms) in length. The dipeptide or tripeptide spacer can be composed of naturally-occurring or coded and/or non-coded or non-naturally occurring amino acids, including, for example, any of the amino acids taught herein. In some embodiments, the spacer comprises an overall negative charge, e.g., comprises one or two negative-charged amino acids. In some embodiments, the dipeptide spacer is selected from the group consisting of: Ala-Ala, β-Ala-β-Ala, Leu-Leu, Pro-Pro, γ-aminobutyric acid-γ-aminobutyric acid, and γ-Glu-γ-Glu. Suitable methods of peptide alkylation via amines, hydroxyls, and thiols are known in the art. For example, a Williamson ether synthesis can be used to form an ether linkage between a hydroxyl group of the peptides and the alkyl group. Also, a nucleophilic substitution reaction of the peptide with an alkyl halide can result in any of an ether, thioether, or amino linkage. The alkyl group of the alkylated peptides can be of any size, e.g., any length carbon chain, and can be linear or branched. In some embodiments, the alkyl group is a C₄ to C₃₀ alkyl. For example, the alkyl group can be any of a C₄ alkyl, C₆ alkyl, C₈ alkyl, C₁₀ alkyl, C₁₂ alkyl, C₁₄ alkyl, C₁₆ alkyl, C₁₈ alkyl, C₂₀ alkyl, C₂₂ alkyl, C₂₄ alkyl, C₂₆ alkyl, C₂₈ alkyl, or a C₃₀ alkyl. In some embodiments, the alkyl group is a C₈ to C₂₀ alkyl, e.g., a C₁₄ alkyl or a C₁₆ alkyl. In some embodiments of the disclosure, the peptide comprises an alkylated amino acid by reacting a nucleophilic, long chain alkane with the peptide, wherein the peptide comprises a leaving group suitable for nucleophilic substitution. In specific aspects, the nucleophilic group of the long chain alkane comprises an amine, hydroxyl, or thiol group (e.g., octadecylamine, tetradecanol, and hexadecanethiol). The leaving group of the peptide can be part of a side chain of an amino acid or can be part of the peptide backbone. Suitable leaving groups include, for example, N-hydroxysuccinimide, halogens, and sulfonate esters. In certain embodiments, the peptide is modified to comprise an alkyl group by reacting the nucleophilic, long chain alkane with a spacer which is attached to the peptide, wherein the spacer comprises the leaving group. In specific aspects, the long chain alkane comprises an amine, hydroxyl, or thiol group. In certain embodiments, the spacer comprising the leaving group can be any spacer discussed herein, e.g., amino acids, dipeptides, tripeptides, hydrophilic bifunctional spacers and hydrophobic bifunctional spacers further comprising a suitable leaving group. With regard to these aspects of the disclosure, in which a long chain alkane is alkylated by the peptides or the spacer, the long chain alkane may be of any size and can comprise any length of carbon chain. The long chain alkane can be linear or branched. In certain aspects, the long chain alkane is a C₄ to C₃₀ alkane. For example, the long chain alkane can be any of a C₄ alkane, C₆ alkane, C₈ alkane, C₁₀ alkane, C₁₂ alkane, C₁₄ alkane, C₁₆ alkane, C₁₈ alkane, C₂₀ alkane, C₂₂ alkane, C₂₄ alkane, C₂₆ alkane, C₂₈ alkane, or a C₃₀ alkane. In some embodiments, the long chain alkane comprises a C₈ to C₂₀ alkane, e.g., a C₁₄ alkane, C₁₆ alkane, or a C₁₈ alkane. Also, in some embodiments, alkylation can occur between the peptides and a cholesterol moiety. For example, the hydroxyl group of cholesterol can displace a leaving group on the long chain alkane to form a cholesterol-peptides product. The alkylated peptides described herein can be further modified to comprise a hydrophilic moiety. In some specific embodiments, the hydrophilic moiety can comprise a polyethylene glycol (PEG) chain. The incorporation of a hydrophilic moiety can be accomplished through any suitable means, such as any of the methods described herein. Alternatively, the alkylated peptides can comprise a spacer, wherein the spacer is both alkylated and modified to comprise the hydrophilic moiety. Nonlimiting examples of suitable spacers include a spacer comprising one or more amino acids selected from the group consisting of Cys, Lys, Orn, homo-Cys, and Ac-Phe.

In some embodiments, the peptide comprises at position 1 or 2, or at both positions 1 and 2, an amino acid which achieves resistance of the peptides to peptidase cleavage. In some embodiments, the peptide comprises at position 1 an amino acid selected from the group consisting of: D-histidine, desaminohistidine, hydroxyl-histidine, acetyl-histidine, homo-histidine, N-methyl histidine, alpha-methyl histidine, imidazole acetic acid, or alpha, alpha-dimethyl imidazole acetic acid (DMIA). In some embodiments, the peptide comprises at position 2 an amino acid selected from the group consisting of: D-serine, D-alanine, valine, glycine, N-methyl serine, N-methyl alanine, or alpha, aminoisobutyric acid. In some embodiments, the peptide comprises at position 2 an amino acid which achieves resistance of the peptide to peptidases and the amino acid which achieves resistance of the peptide to peptidases is not D-serine. In some embodiments, this covalent bond is an intramolecular bridge other than a lactam bridge. For example, suitable covalent bonding methods include any one or more of olefin metathesis, lanthionine-based cyclization, disulfide bridge or modified sulfur-containing bridge formation, the use of α,ω-diaminoalkane tethers, the formation of metal-atom bridges, and other means of peptide cyclization.

In some embodiments, the peptide is modified by amino acid substitutions and/or additions that introduce a charged amino acid into the C-terminal portion of the analog. In some embodiments, such modifications enhance stability and solubility. As used herein the term “charged amino acid” or “charged residue” refers to an amino acid that comprises a side chain that is negative-charged (i.e., de-protonated) or positive-charged (i.e., protonated) in aqueous solution at physiological pH. In some aspects, these amino acid substitutions and/or additions that introduce a charged amino acid modifications may be at a C-terminal position. In some embodiments, one, two or three (and in some instances, more than three) charged amino acids may be introduced at the C-terminal position. In exemplary embodiments, one, two or all of the charged amino acids may be negative-charged. The negative-charged amino acid in some embodiments is aspartic acid, glutamic acid, cysteic acid, homocysteic acid, or homoglutamic acid. In some aspects, these modifications increase solubility.

In accordance with some embodiments, the peptides disclosed herein may be modified by truncation of the C-terminus by one or two amino acid residues. In this regard, the peptides can comprise the sequences (SEQ ID NO: 1-64 and 69-79), optionally with any of the additional modifications described herein.

In some embodiments, the peptide comprises a modified SEQ ID NO: 1-64 and 69-79 in which the carboxylic acid of the C-terminal amino acid is replaced with a charge-neutral group, such as an amide or ester. Accordingly, in some embodiments, the peptide is an amidated peptide, such that the C-terminal residue comprises an amide in place of the alpha carboxylate of an amino acid. As used herein a general reference to a peptide or analog is intended to encompass peptides that have a modified amino terminus, a modified carboxy terminus, or modifications of both amino and carboxy termini. For example, an amino acid chain composing an amide group in place of the terminal carboxylic acid is intended to be encompassed by an amino acid sequence designating the standard amino acids.

In accordance with some embodiments, the peptides disclosed herein may be modified by conjugation on at least one amino acid residue. In this regard, the peptides can comprise the sequences (SEQ ID NOs: 1-64 and 69-79), optionally with any of the additional conjugations described herein.

The disclosure further provides conjugates comprising one or more of the peptides described herein conjugated to a heterologous moiety. As used herein, the term “heterologous moiety” is synonymous with the term “conjugate moiety” and refers to any molecule (chemical or biochemical, naturally-occurring or non-coded) which is different from the peptides described herein. Exemplary conjugate moieties that can be linked to any of the analogs described herein include but are not limited to a heterologous peptide or polypeptide (including for example, a plasma protein), a targeting agent, an immunoglobulin or portion thereof (e.g., variable region, CDR, or Fc region), a diagnostic label such as a radioisotope, fluorophore or enzymatic label, a polymer including water soluble polymers, or other therapeutic or diagnostic agents. In some embodiments a conjugate is provided comprising a peptide and a plasma protein, wherein the plasma protein is selected from the group consisting of albumin, transferrin, fibrinogen and globulins. In some embodiments the plasma protein moiety of the conjugate is albumin or transferrin.

The conjugate in some embodiments comprises one or more of the peptides described herein and one or more of: a different peptide (which is distinct from the peptides described herein), a polypeptide, a nucleic acid molecule, an antibody or fragment thereof, a polymer, a quantum dot, a small molecule, a toxin, a diagnostic agent, a carbohydrate, an amino acid. In some embodiments, the heterologous moiety is a polymer. In some embodiments, the polymer is selected from the group consisting of: polyamides, polycarbonates, polyalkylenes and derivatives thereof including, polyalkylene glycols, polyalkylene oxides, polyalkylene terepthalates, polymers of acrylic and methacrylic esters, including poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate), polyvinyl polymers including polyvinyl alcohols, polyvinyl ethers, polyvinyl esters, polyvinyl halides, poly(vinyl acetate), and polyvinylpyrrolidone, polyglycolides, polysiloxanes, polyurethanes and co-polymers thereof, celluloses including alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulphate sodium salt, polypropylene, polyethylenes including poly(ethylene glycol), poly(ethylene oxide), and poly(ethylene terephthalate), and polystyrene. In some aspects, the polymer is a biodegradable polymer, including a synthetic biodegradable polymer (e.g., polymers of lactic acid and glycolic acid, polyanhydrides, poly(ortho)esters, polyurethanes, poly(butic acid), poly(valeric acid), and poly(lactide-cocaprolactone)), and a natural biodegradable polymer (e.g., alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins (e.g., zein and other prolamines and hydrophobic proteins)), as well as any copolymer or mixture thereof. In general, these materials degrade either by enzymatic hydrolysis or exposure to water in vivo, by surface or bulk erosion. In some aspects, the polymer is a bioadhesive polymer, such as a bioerodible hydrogel described by H. Sawhney, et al., [Macromolecules, 1993, 26, 581-587] the teachings of which are incorporated herein, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

In some embodiments, the polymer is a water-soluble polymer or a hydrophilic polymer. Hydrophilic polymers are further described herein under “Hydrophilic Moieties.” Suitable water-soluble polymers are known in the art and include, for example, polyvinylpyrrolidone, hydroxypropyl cellulose (HPC; Klucel), hydroxypropyl methylcellulose (HPMC; Methocel), nitrocellulose, hydroxypropyl ethylcellulose, hydroxypropyl butylcellulose, hydroxypropyl pentylcellulose, methyl cellulose, ethylcellulose (Ethocel), hydroxyethyl cellulose, various alkyl celluloses and hydroxyalkyl celluloses, various cellulose ethers, cellulose acetate, carboxymethyl cellulose, sodium carboxymethyl cellulose, calcium carboxymethyl cellulose, vinyl acetate/crotonic acid copolymers, poly-hydroxyalkyl methacrylate, hydroxymethyl methacrylate, methacrylic acid copolymers, polymethacrylic acid, polymethylmethacrylate, maleic anhydride/methyl vinyl ether copolymers, poly vinyl alcohol, sodium and calcium polyacrylic acid, polyacrylic acid, acidic carboxy polymers, carboxypolymethylene, carboxyvinyl polymers, polyoxyethylene polyoxypropylene copolymer, polymethylvinylether co-maleic anhydride, carboxymethylamide, potassium methacrylate divinylbenzene co-polymer, polyoxyethyleneglycols, polyethylene oxide, and derivatives, salts, and combinations thereof. In specific embodiments, the polymer is a polyalkylene glycol, including, for example, polyethylene glycol (PEG).

In some embodiments, the heterologous moiety is a carbohydrate. In some embodiments, the carbohydrate is a monosaccharide (e.g., glucose, galactose, fructose), a disaccharide (e.g., sucrose, lactose, maltose), an oligosaccharide (e.g., raffinose, stachyose), a polysaccharide (a starch, amylase, amylopectin, cellulose, chitin, callose, laminarin, xylan, mannan, fucoidan, galactomannan.

In some embodiments, the heterologous moiety is a lipid. The lipid, in some embodiments, is a fatty acid, eicosanoid, prostaglandin, leukotriene, thromboxane, N-acyl ethanolamine), glycerolipid (e.g., mono-, di-, tri-substituted glycerols), glycerophospholipid (e.g., phosphatidylcholine, phosphatidylinositol, phosphatidylethanolamine, phosphatidylserine), sphingolipid (e.g., sphingosine, ceramide), sterol lipid (e.g., steroid, cholesterol), prenol lipid, saccharolipid, or a polyketide, oil, wax, cholesterol, sterol, fat-soluble vitamin, monoglyceride, diglyceride, triglyceride, a phospholipid.

In some embodiments, the heterologous moiety is attached via non-covalent or covalent bonding to the peptide of the present disclosure. In certain aspects, the heterologous moiety is attached to the peptide of the present disclosure via a linker. Linkage can be accomplished by covalent chemical bonds, physical forces such electrostatic, hydrogen, ionic, van der Waals, or hydrophobic or hydrophilic interactions. A variety of non-covalent coupling systems may be used, including biotin-avidin, ligand/receptor, enzyme/substrate, nucleic acid/nucleic acid binding protein, lipid/lipid binding protein, cellular adhesion molecule partners; or any binding partners or fragments thereof which have affinity for each other. The peptide in some embodiments is linked to conjugate moieties via direct covalent linkage by reacting targeted amino acid residues of the analog with an organic derivatizing agent that is capable of reacting with selected side chains or the N- or C-terminal residues of these targeted amino acids. Reactive groups on the analog or conjugate moiety include, e.g., an aldehyde, amino, ester, thiol, α-haloacetyl, maleimido or hydrazino group. Derivatizing agents include, for example, maleimidobenzoyl sulfosuccinimide ester (conjugation through cysteine residues), N-hydroxysuccinimide (through lysine residues), glutaraldehyde, succinic anhydride or other agents known in the art. Alternatively, the conjugate moieties can be linked to the analog indirectly through intermediate carriers, such as polysaccharide or polypeptide carriers. Examples of polysaccharide carriers include aminodextran. Examples of suitable polypeptide carriers include polylysine, polyglutamic acid, polyaspartic acid, co-polymers thereof, and mixed polymers of these amino acids and others, e.g., serines, to confer desirable solubility properties on the resultant loaded carrier. Cysteinyl residues are most commonly reacted with α-haloacetates (and corresponding amines), such as chloroacetic acid, chloroacetamide to give carboxymethyl or carboxyamidomethyl derivatives. Cysteinyl residues also may be derivatized by reaction with bromotrifluoroacetone, alpha-bromo-β-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri nitrophenol, or chloro-7-nitrobenzo-2-oxa-1,3-diazole. Histidyl residues may be derivatized by reaction with diethylpyrocarbonate at pH 5.5-7.0 because this agent is relatively specific for the histidyl side chain. Para-bromophenacyl bromide also is useful; the reaction is preferably performed in 0.1 M sodium cacodylate at pH 6.0. Lysinyl and amino-terminal residues may be reacted with succinic or other carboxylic acid anhydrides. Derivatization with these agents has the effect of reversing the charge of the lysinyl residues. Other suitable reagents for derivatizing alpha-amino-containing residues include imidoesters such as methyl picolinimidate, pyridoxal phosphate, pyridoxal, chloroborohydride, trinitrobenzenesulfonic acid, O-methylisourea, 2,4-pentanedione, and transaminase-catalyzed reaction with glyoxylate. Arginyl residues may be modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKa of the guanidine functional group. Furthermore, these reagents may react with the groups of lysine as well as the arginine epsilon-amino group. The specific modification of tyrosyl residues may be made, with particular interest in introducing spectral labels into tyrosyl residues by reaction with aromatic diazonium compounds or tetranitromethane. Most commonly, N-acetylimidizole and tetranitromethane are used to form 0-acetyl tyrosyl species and 3-nitro derivatives, respectively. Carboxyl side groups (aspartyl or glutamyl) may be selectively modified by reaction with carbodiimides (R—N═C═N—R′), where R and R′ are different alkyl groups, such as 1-cyclohexyl-3-(2-morpholinyl-4-ethyl) carbodiimide or 1-ethyl-3-(4-azonia-4,4-dimethylpentyl) carbodiimide. Furthermore, aspartyl and glutamyl residues may be converted to asparaginyl and glutaminyl residues by reaction with ammonium ions. Other modifications include hydroxylation of proline and lysine, phosphorylation of hydroxyl groups of seryl or threonyl residues, methylation of the alpha-amino groups of lysine, arginine, and histidine side chains (T. E. Creighton, Proteins: Structure and Molecular Properties, W.H. Freeman & Co., San Francisco, pp. 79-86 (1983)), deamidation of asparagine or glutamine, acetylation of the N-terminal amine, and/or amidation or esterification of the C-terminal carboxylic acid group. Another type of covalent modification involves chemically or enzymatically coupling glycosides to the peptide. Sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of tyrosine, or tryptophan, or (f) the amide group of glutamine. These methods are described in WO87/05330 published 11 Sep. 1987, and in Aplin and Wriston, CRC Crit. Rev. Biochem., pp. 259-306 (1981). In some embodiments, the peptide is conjugated to a heterologous moiety via covalent linkage between a side chain of an amino acid of the peptides and the heterologous moiety. In some aspects, the amino acid covalently linked to a heterologous moiety (e.g., the amino acid comprising a heterologous moiety) is a Cys, Lys, Orn, homo-Cys, or Ac-Phe, and the side chain of the amino acid is covalently bonded to a heterologous moiety. In some embodiments, the conjugate comprises a linker that joins the peptide to the heterologous moiety. In some aspects, the linker comprises a chain of atoms from 1 to about 60, or 1 to 30 atoms or longer, 2 to 5 atoms, 2 to 10 atoms, 5 to 10 atoms, or 10 to 20 atoms long. In some embodiments, the chain atoms may be all carbon atoms. In some embodiments, the chain atoms in the backbone of the linker may be selected from the group consisting of C, O, N, and S. Chain atoms and linkers may be selected according to their expected solubility (hydrophilicity) so as to provide a more soluble conjugate. In some embodiments, the linker provides a functional group that is subject to cleavage by an enzyme or other catalyst or hydrolytic conditions found in the target tissue or organ or cell. In some embodiments, the length of the linker is long enough to reduce the potential for steric hindrance. If the linker is a covalent bond or a peptidyl bond and the conjugate is a polypeptide, the entire conjugate can be a fusion protein. Such peptidyl linkers may be any length. Exemplary linkers may be from about 1 to 50 amino acids in length, 5 to 50, 3 to 5, 5 to 10, 5 to 15, or 10 to 30 amino acids in length. Such fusion proteins may alternatively be produced by recombinant genetic engineering methods known to one of ordinary skill in the art.

As noted above, in some embodiments, the peptides may be conjugated, e.g., fused to an immunoglobulin or portion thereof (e.g., variable region, CDR, or Fc region). Known types of immunoglobulins (Ig) include IgG, IgA, IgE, IgD or IgM. The Fc region is a C-terminal region of an Ig heavy chain, which is responsible for binding to Fc receptors that carry out activities such as recycling (which results in prolonged half-life), antibody dependent cell-mediated cytotoxicity (ADCC), and complement dependent cytotoxicity (CDC). For example, according to some definitions the human IgG heavy chain Fc region stretches from Cys226 to the C-terminus of the heavy chain. The “hinge region” generally extends from Glu216 to Pro230 of human IgG1 (hinge regions of other IgG isotypes may be aligned with the IgG1 sequence by aligning the cysteines involved in cysteine bonding). The Fc region of an IgG includes two constant domains, CH₂ and CH₃. The CH₂ domain of a human IgG Fc region usually extends from amino acids 231 to amino acid 341. The CH₃ domain of a human IgG Fc region usually extends from amino acids 342 to 447. References made to amino acid numbering of immunoglobulins or immunoglobulin fragments, or regions, are all based on Kabat et al. 1991, Sequences of Proteins of Immunological Interest, U.S. Department of Public Health, Bethesda, Md. In related embodiments, the Fc region may comprise one or more native or modified constant regions from an immunoglobulin heavy chain, other than CH₁, for example, the CH₂ and CH₃ regions of IgG and IgA, or the CH₃ and CH₄ regions of IgE. Suitable conjugate moieties include portions of immunoglobulin sequence that include the FcRn binding site. FcRn, a salvage receptor, is responsible for recycling immunoglobulins and returning them to circulation in blood. The region of the Fc portion of IgG that binds to the FcRn receptor has been described based on X-ray crystallography (Burmeister et al. 1994, Nature 372:379). The major contact area of the Fc with the FcRn is near the junction of the CH₂ and CH₃ domains. Fc-FcRn contacts are all within a single Ig heavy chain. The major contact sites include amino acid residues 248, 250-257, 272, 285, 288, 290-291, 308-311, and 314 of the CH₂ domain and amino acid residues 385-387, 428, and 433-436 of the CH₃ domain. Some conjugate moieties may or may not include FcγR binding site(s). FcγR are responsible for ADCC and CDC. Examples of positions within the Fc region that make a direct contact with FcγR are amino acids 234-239 (lower hinge region), amino acids 265-269 (B/C loop), amino acids 297-299 (C′/E loop), and amino acids 327-332 (F/G) loop (Sondermann et al., Nature 406: 267-273, 2000). The lower hinge region of IgE has also been implicated in the FcRI binding (Henry, et al., Biochemistry 36, 15568-15578, 1997). Residues involved in IgA receptor binding are described in Lewis et al., (J Immunol. 175:6694-701, 2005). Amino acid residues involved in IgE receptor binding are described in Sayers et al. (J Biol Chem. 279(34):35320-5, 2004). Amino acid modifications may be made to the Fc region of an immunoglobulin. Such variant Fc regions comprise at least one amino acid modification in the CH₃ domain of the Fc region (residues 342-447) and/or at least one amino acid modification in the CH₂ domain of the Fc region (residues 231-341). Mutations believed to impart an increased affinity for FcRn include T256A, T307A, E380A, and N434A (Shields et al. 2001, J. Biol. Chem. 276:6591). Other mutations may reduce binding of the Fc region to FcγRI, FcγRIIA, FcγRIIB, and/or FcγRIIIA without significantly reducing affinity for FcRn. For example, substitution of the Asn at position 297 of the Fc region with Ala or another amino acid removes a highly conserved N-glycosylation site and may result in reduced immunogenicity with concomitant prolonged half-life of the Fc region, as well as reduced binding to FcγRs (Routledge et al. 1995, Transplantation 60:847; Friend et al. 1999, Transplantation 68:1632; Shields et al. 1995, J. Biol. Chem. 276:6591). Amino acid modifications at positions 233-236 of IgG1 have been made that reduce binding to FcγRs (Ward and Ghetie 1995, Therapeutic Immunology 2:77 and Armour et al. 1999, Eur. J. Immunol. 29:2613). Some exemplary amino acid substitutions are described in U.S. Pat. Nos. 7,355,008 and 7,381,408, each incorporated by reference herein in its entirety. In certain embodiments, a peptide described herein is inserted into a loop region within the immunoglobulin molecule. In other embodiments, a peptide described herein replaces one or more amino acids of a loop region within the immunoglobulin molecule.

The peptides described herein can be further modified to improve its solubility and stability in aqueous solutions at physiological pH, while retaining the biological activity. Hydrophilic moieties such as PEG groups can be attached to the analogs under any suitable conditions used to react a protein with an activated polymer molecule. Any means known in the art can be used, including via acylation, reductive alkylation, Michael addition, thiol alkylation or other chemoselective conjugation/ligation methods through a reactive group on the PEG moiety (e.g., an aldehyde, amino, ester, thiol, α-haloacetyl, maleimido or hydrazino group) to a reactive group on the target compound (e.g., an aldehyde, amino, ester, thiol, α-haloacetyl, maleimido or hydrazino group). Activating groups which can be used to link the water soluble polymer to one or more proteins include without limitation sulfone, maleimide, sulfhydryl, thiol, triflate, tresylate, azidirine, oxirane, 5-pyridyl, and alpha-halogenated acyl group (e.g., alpha-iodo acetic acid, alpha-bromoacetic acid, alpha-chloroacetic acid). If attached to the analog by reductive alkylation, the polymer selected should have a single reactive aldehyde so that the degree of polymerization is controlled. See, for example, Kinstler et al., Adv. Drug. Delivery Rev. 54: 477-485 (2002); Roberts et al., Adv. Drug Delivery Rev. 54: 459-476 (2002); and Zalipsky et al., Adv. Drug Delivery Rev. 16: 157-182 (1995). In specific aspects, an amino acid residue of the peptides having a thiol is modified with a hydrophilic moiety such as PEG. In some embodiments, the thiol is modified with maleimide-activated PEG in a Michael addition reaction to result in a PEGylated analog comprising a thioether linkage. In some embodiments, the thiol is modified with a haloacetyl-activated PEG in a nucleophilic substitution reaction to result in a PEGylated analog comprising a thioether linkage. Suitable hydrophilic moieties include polyethylene glycol (PEG), polypropylene glycol, polyoxyethylated polyols (e.g., POG), polyoxyethylated sorbitol, polyoxyethylated glucose, polyoxyethylated glycerol (POG), polyoxyalkylenes, polyethylene glycol propionaldehyde, copolymers of ethylene glycol/propylene glycol, monomethoxy-polyethylene glycol, mono-(C₁-C₁₀) alkoxy- or aryloxy-polyethylene glycol, carboxymethylcellulose, polyacetals, polyvinyl alcohol (PVA), polyvinyl pyrrolidone, poly-1,3-dioxolane, poly-1,3,6-trioxane, ethylene/maleic anhydride copolymer, poly (.beta.-amino acids) (either homopolymers or random copolymers), poly(n-vinyl pyrrolidone)polyethylene glycol, propropylene glycol homopolymers (PPG) and other polyalkylene oxides, polypropylene oxide/ethylene oxide copolymers, colonic acids or other polysaccharide polymers, Ficoll or dextran and mixtures thereof. Dextrans are polysaccharide polymers of glucose subunits, predominantly linked by α1-6 linkages. Dextran is available in many molecular weight ranges, e.g., about 1 kD to about 100 kD, or from about 5, 10, 15 or 20 kD to about 20, 30, 40, 50, 60, 70, 80 or 90 kD. Linear or branched polymers are contemplated. Resulting preparations of conjugates may be essentially monodisperse or polydisperse, and may have about 0.5, 0.7, 1, 1.2, 1.5 or 2 polymer moieties per analog.

In some embodiments, the peptide is conjugated to a hydrophilic moiety via covalent linkage between a side chain of an amino acid of the peptide and the hydrophilic moiety. In some embodiments, the peptide is conjugated to a hydrophilic moiety via the side chain of an amino acid, a position within a C-terminal extension, or the C-terminal amino acid, or a combination of these positions. In some aspects, the amino acid covalently linked to a hydrophilic moiety (e.g., the amino acid comprising a hydrophilic moiety) is a Cys, Lys, Orn, homo-Cys, or Ac-Phe, and the side chain of the amino acid is covalently bonded to a hydrophilic moiety (e.g., PEG). In some embodiments, the conjugate of the present disclosure comprises the peptide fused to an accessory analog which is capable of forming an extended conformation similar to chemical PEG (e.g., a recombinant PEG (rPEG) molecule), such as those described in International Patent Application Publication No. WO 2009/023270 and U.S. Patent Application Publication No. US 2008/0286808. The rPEG molecule in some aspects is a polypeptide comprising one or more of glycine, serine, glutamic acid, aspartic acid, alanine, or proline. In some aspects, the rPEG is a homopolymer, e.g., poly-glycine, poly-serine, poly-glutamic acid, poly-aspartic acid, poly-alanine, or poly-proline. In other embodiments, the rPEG comprises two types of amino acids repeated, e.g., poly(Gly-Ser), poly(Gly-Glu), poly(Gly-Ala), poly(Gly-Asp), poly(Gly-Pro), poly(Ser-Glu), etc. In some aspects, the rPEG comprises three different types of amino acids, e.g., poly(Gly-Ser-Glu). In specific aspects, the rPEG increases the half-life of the peptide. In some aspects, the rPEG comprises a net positive or net negative charge. The rPEG in some aspects lacks secondary structure. In some embodiments, the rPEG is greater than or equal to 10 amino acids in length and in some embodiments is about 40 to about 50 amino acids in length. The accessory peptide in some aspects is fused to the N- or C-terminus of the peptide of the present disclosure through a peptide bond or a proteinase cleavage site, or is inserted into the loops of the peptide of the present disclosure. The rPEG in some aspects comprises an affinity tag or is linked to a PEG that is greater than 5 kDa. In some embodiments, the rPEG confers the peptide of the present disclosure with an increased hydrodynamic radius, serum half-life, protease resistance, or solubility and in some aspects confers the analog with decreased immunogenicity.

The peptides comprising the sequences (SEQ ID NO: 1-64 and 69-79), optionally with any of the conjugations described herein are contemplated as an embodiment.

The disclosure further provides multimers or dimers of the peptides disclosed herein, including homo- or hetero-multimers or homo- or hetero-dimers. Two or more of the analogs can be linked together using standard linking agents and procedures known to those skilled in the art. For example, dimers can be formed between two peptides through the use of bifunctional thiol crosslinkers and bi-functional amine crosslinkers, particularly for the analogs that have been substituted with cysteine, lysine ornithine, homocysteine or acetyl phenylalanine residues. The dimer can be a homodimer or alternatively can be a heterodimer. In certain embodiments, the linker connecting the two (or more) analogs is PEG, e.g., a 5 kDa PEG, 20 kDa PEG. In some embodiments, the linker is a disulfide bond. For example, each monomer of the dimer may comprise a Cys residue (e.g., a terminal or internally positioned Cys) and the sulfur atom of each Cys residue participates in the formation of the disulfide bond. In some aspects, the monomers may be connected via terminal amino acids (e.g., N-terminal or C-terminal), via internal amino acids, or via a terminal amino acid of at least one monomer and an internal amino acid of at least one other monomer. In specific aspects, the monomers are not connected via an N-terminal amino acid. In some aspects, the monomers of the multimer may be attached together in a “tail-to-tail” orientation in which the C-terminal amino acids of each monomer may be attached together.

Peptides disclosed herein may be made in a variety of ways. Suitable methods of de novo synthesizing peptides are described in, for example, Merrifield, J. Am. Chem. Soc, 85, 2149 (1963); Davis et al., Biochem. Intl., 10, 394-414 (1985); Larsen et al., J. Am. Chem. Soc, 115, 6247 (1993); Smith et al., J. Peptide Protein Res., 44, 183 (1994); O'Donnell et al., J. Am. Chem. Soc, 118, 6070 (1996); Stewart and Young, Solid Phase Peptide Synthesis, Freeman (1969); Finn et al., The Proteins, 3 ed., vol. 2, pp. 105-253 (1976); Erickson et al., The Proteins, 3^{rd} ed., vol. 2, pp. 257-527 (1976); and Chan et al., Fmoc Solid Phase Peptide Synthesis, Oxford University Press, Oxford, United Kingdom, 2005. The disclosure contemplates synthetic peptides. Methods of making the peptides are themselves embodiments of the invention.

Alternatively, the peptide can be expressed recombinantly by introducing a nucleic acid that comprises or consists of a nucleotide sequence encoding a peptide into host cells, which may be cultured to express the encoded peptide using standard recombinant methods. See, for instance, Sambrook et al., Molecular Cloning: A Laboratory Manual. 3rd ed., Cold Spring Harbor Press, Cold Spring Harbor, N.Y. 2001; and Ausubel et al., Current Protocols in Molecular Biology, Greene Publishing Associates and John Wiley & Sons, N.Y., 1994. Such peptides may be purified from the culture media or cell pellets.

In some embodiments, the peptides of the disclosure can be isolated. In some embodiments, the peptides of the disclosure may be purified. It is recognized that “purity” is a relative term, and not to be necessarily construed as absolute purity or absolute enrichment or absolute selection. In some aspects, the purity is at least or about 50%, is at least or about 60%, at least or about 70%, at least or about 80%, or at least or about 90% (e.g., at least or about 91%, at least or about 92%, at least or about 93%, at least or about 94%, at least or about 95%, at least or about 96%, at least or about 97%, at least or about 98%, at least or about 99% or is approximately 100%.

In some embodiments, the peptides described herein can be commercially synthesized by companies, such as Genscript (Piscataway, N.J.), New England Peptide (Gardner, Mass.), and CPC Scientific (Sunnyvale, Calif.), Peptide Technologies Corp. (Gaithersburg, Md.), and Multiple Peptide Systems (San Diego, Calif.). In this respect, the peptides can be synthetic, recombinant, isolated, and/or purified.

The peptides of the present disclosure can be provided in accordance with one embodiment as part of a kit. Accordingly, in some embodiments, a kit for administering a peptide, to a patient in need thereof is provided wherein the kit comprises a peptide as described herein.

In one embodiment the kit is provided with a device for administering the composition to a patient, e.g., syringe needle, pen device, jet injector or another needle-free injector. The kit may alternatively or in addition include one or more containers, e.g., vials, tubes, bottles, single or multi-chambered pre-filled syringes, cartridges, infusion pumps (external or implantable), jet injectors, pre-filled pen devices and the like, optionally containing the peptide in a lyophilized form or in an aqueous solution. The kits in some embodiments comprise instructions for use. In accordance with one embodiment the device of the kit is an aerosol dispensing device, wherein the composition is prepackaged within the aerosol device. In another embodiment the kit comprises a syringe and a needle, and in one embodiment the sterile composition is prepackaged within the syringe.

A further embodiment includes a process of treating a disease comprising one or more of prescribing, selling or advertising to sell, purchasing, instructing to self-administer, or administering a peptide described herein, wherein the peptide has been approved by a regulatory agency for the treatment of a condition, to a subject in need of treatment.

A further embodiment includes a method of supplying a peptide for treating a disease, said method comprises reimbursing a physician, a formulary, a patient or an insurance company for the sale of said peptide.

### Definitions

The terms “peptide” refers to a molecule comprising two or more amino acid residues joined to each other by peptide bonds. These terms encompass, e.g., native and artificial proteins, protein fragments and polypeptide analogs (such as muteins, variants, and fusion proteins) of a protein sequence as well as post-translationally, or otherwise covalently or non-covalently, modified peptides. A peptide may be monomeric or polymeric. In certain embodiments, “peptides” are chains of amino acids whose alpha carbons may be linked through peptide bonds. The terminal amino acid at one end of the chain (amino terminal) therefore has a free amino group, while the terminal amino acid at the other end of the chain (carboxy terminal) has a free carboxyl group. As used herein, the term “amino terminus” (abbreviated N-terminus) refers to the free α-amino group on an amino acid at the amino terminal of a peptide or to the α-amino group (imino group when participating in a peptide bond) of an amino acid at any other location within the peptide. Similarly, the term “carboxy terminus” refers to the free carboxyl group on the carboxy terminus of a peptide or the carboxyl group of an amino acid at any other location within the peptide. Peptides also include essentially any polyamino acid including, but not limited to, peptide mimetics such as amino acids joined by an ether as opposed to an amide bond.

The term “therapeutic peptide” refers to peptides or analogs or fragments or variants thereof, having one or more therapeutic and/or biological activities.

The term “analog” as used herein describes a peptide comprising one or more amino acid modifications, such as but not limited to substitution and/or one or more deletion and/or one or more addition of any one of the amino acid residues for any natural or unnatural amino acid, synthetic amino acids or peptidomimetics and/or the attachment of a side chain to any one of the natural or unnatural amino acids, synthetic amino acids or peptidomimetics at any available position. The addition or deletion of amino acid residues can take place at the N-terminal of the peptide and/or at the C-terminal of the peptide.

In some embodiments, the analog has 1, 2, 3, 4, or 5 such modifications. In some embodiments, the analog retains biological activity of the original peptide. In some embodiments, the analog is a competitive or non-competitive inhibitor of the original peptide.

Peptide sequences are indicated using standard one- or three-letter abbreviations. Unless otherwise indicated, peptide sequences have their amino termini at the left and their carboxy termini at the right, a particular section of a peptide can be designated by amino acid residue number such as amino acids 3 to 6, or by the actual residue at that site such as Met3 to Gly6. A particular peptide sequence also can be described by explaining how it differs from a reference sequence.

When used herein the term “natural amino acid” is an amino acid (with the usual three letter codes & one letter codes in parenthesis) selected from the group consisting of: Glycine (Gly & G), proline (Pro & P), alanine (Ala & A), valine (Val & V), leucine (Leu & L), isoleucine (Ile & I), methionine (Met & M), cysteine (Cys & C), phenylalanine (Phe & F), tyrosine (Tyr & Y), tryptophan (Trp & W), histidine (His & H), lysine (Lys & K), arginine (Arg & R), glutamine (Gin & Q), asparagine (Asn & N), glutamic acid (Glu & E), aspartic acid (Asp & D), serine (Ser & S) and threonine (Thr & T). If anywhere herein, reference is made to a peptide, analog or derivative or peptides comprising or not comprising G, P, A, V, L, I, M, C, F, Y, H, K, R, Q, N, E, D, S or T, without specifying further, amino acids are meant. If not otherwise indicated amino acids indicated with a single letter code in CAPITAL letters indicate the L-isoform, if however, the amino acid is indicated with a lower case letter, this amino acid is used/applied as it's D-form. Such D-forms and other non-conservative amino acid substitutions previously defined are included in a definition of unnatural amino acids.

If, due to typing errors, there are deviations from the commonly used codes, the commonly used codes apply. The amino acids present in the peptides are, preferably, amino acids which can be coded for by a nucleic acid. As is apparent from the above examples, amino acid residues may be identified by their full name, their one-letter code, and/or their three-letter code. These three ways are fully equivalent.

A “non-conservative amino acid substitution” also refers to the substitution of a member of one of these classes for a member from another class. In making such changes, according to certain embodiments, the hydropathic index of amino acids may be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). The importance of the hydropathic amino acid index in conferring interactive biological function on a protein is understood in the art (see, for example, Kyte et al., 1982, J. Mol. Biol. 157:105-131). It is known that certain amino acids may be substituted for other amino acids having a similar hydropathic index or score and still retain a similar biological activity. In making changes based upon the hydropathic index, in certain embodiments, the substitution of amino acids whose hydropathic indices are within ±2 is included. In certain embodiments, those that are within ±1 are included, and in certain embodiments, those within ±0.5 are included. It is also understood in the art that the substitution of like amino acids can be made effectively on the basis of hydrophilicity, particularly where the biologically functional protein or peptide thereby created is intended for use in immunological embodiments, as disclosed herein. In certain embodiments, the greatest local average hydrophilicity of a protein, as governed by the hydrophilicity of its adjacent amino acids, correlates with its immunogenicity and antigenicity, i.e., with a biological property of the protein. The following hydrophilicity values have been assigned to these amino acid residues: arginine (+3.0); lysine (+3.0); aspartate (+3.0.+−.1); glutamate (+3.0.+−.1); serine (+0.3); asparagine (+0.2); glutamine (+0.2); glycine (0); threonine (−0.4); proline (−0.5.+−.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments, the substitution of amino acids whose hydrophilicity values are within ±2 is included, in certain embodiments, those that are within ±1 are included, and in certain embodiments, those within ±0.5 are included.

Other amino acid substitutions are set forth in Table 3.

TABLE 3

| Original | | Preferred |
|---|---|---|
| Residues | Substitutions | Substitutions |
| Ala | Val, Leu, Ile | Val |
| Arg | Lys, Gln, Asn | Lys |
| Asn | Gln | Gln |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, Phe, Norleucine | Leu |
| Leu | Norleucine, Ile, Val, Met, Ala, Phe | Ile |
| Lys | Arg, Gln, Asn, 1,4-Diamino-butyric Acid | Arg |
| Met | Leu, Phe, Ile | Leu |
| Phe | Leu, Val, Ile, Ala, Tyr | Leu |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, Ala, Norleucine | Leu |

As used herein the term “charged amino acid” or “charged residue” refers to an amino acid that comprises a side chain that is negative-charged (i.e., de-protonated) or positive-charged (i.e., protonated) in aqueous solution at physiological pH. For example, negative-charged amino acids include aspartic acid, glutamic acid, cysteic acid, homocysteic acid, and homoglutamic acid, whereas positive-charged amino acids include arginine, lysine and histidine. Charged amino acids include the charged amino acids among the 20 coded amino acids, as well as atypical or non-naturally occurring or non-coded amino acids.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

When ranges of values are disclosed, and the notation “from n₁ . . . to n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar)” which is intended to include 1 μM, 3 μM, and everything in between to any number of significant figures (e.g., 1.255 μM, 2.1 μM, 2.9999 μM, etc.).

The term “acyl” as used herein, alone or in combination, refers to a carbonyl attached to an alkenyl, alkyl, aryl, cycloalkyl, heteroaryl, heterocycloalkyl, or any other moiety were the atom attached to the carbonyl is carbon. An “acetyl” group, which is a type of acyl, refers to a —C(O)CH₃ group. An “alkylcarbonyl” or “alkanoyl” group refers to an alkyl group attached to the parent molecular moiety through a carbonyl group. Examples of such groups include methylcarbonyl and ethyl carbonyl. Examples of acyl groups include formyl, alkanoyl and aroyl.

The term “amino acid” as used herein, alone or in combination, means a substituent of the form —R^{x}—NH—CH(R^{y})C(═O)OH, wherein R^{x} is typically hydrogen, but may be cyclized with N (for example, as in the case of the amino acid proline), and R^{y} is selected from the group consisting of hydrogen, alkyl, heteroalkyl, cycloalkyl, heterocycloalkyl, aryl, heteroaryl, amino, amido, cycloalkylalkyl, heterocycloalkylalkyl, arylalkyl, heteroarylalkyl, aminoalkyl, amidoalkyl, hydroxyalkyl, thiol, thioalkyl, alkylthioalkyl, and alkylthio, any of which may be optionally substituted. The term “amino acid” includes all naturally occurring amino acids as well as synthetic analogs.

The term “disease” as used herein is intended to be generally synonymous, and is used interchangeably with, the terms “disorder” and “condition” (as in medical condition), in that all reflect an abnormal condition of the body or of one of its parts that impairs normal functioning and is typically manifested by distinguishing signs and symptoms.

As used herein the term “acidic amino acid” refers to an amino acid that comprises a second acidic moiety (other than the carboxylic acid of the amino acid), including for example, a carboxylic acid or sulfonic acid group.

As used herein, the term “acylated amino acid” refers to an amino acid comprising an acyl group which is non-native to a naturally-occurring amino acid, regardless of the means by which it is produced (e.g. acylation prior to incorporating the amino acid into a peptide, or acylation after incorporation into a peptide).

As used herein the term “alkylated amino acid” refers to an amino acid comprising an alkyl group which is non-native to a naturally-occurring amino acid, regardless of the means by which it is produced. Accordingly, the acylated amino acids and alkylated amino acids of the present disclosures are non-coded amino acids.

A skilled artisan will be able to determine active variants or analogs of peptides as set forth herein using well-known techniques. In certain embodiments, one skilled in the art may identify suitable areas of the molecule that may be changed without destroying activity by targeting regions not believed to be important for activity. In other embodiments, the skilled artisan can identify residues and portions of the molecules that are conserved among similar peptides. In further embodiments, even areas that may be important for biological activity or for structure may be subject to conservative amino acid substitutions without destroying the biological activity or without adversely affecting the peptide structure. Changes in caspase activity in cells treated with a test compounds are well known to be an indicator of potential therapeutic utility. Regardless of whether caspases have been definitively implicated in the etiology or pathological consequences of a disease, a decrease in caspase activity has been associated with amelioration of the symptoms of several conditions caused by inappropriate apoptotic cell death, including diabetes, cardiovascular disease, detrimental hepatocyte apoptosis, ischemia reperfusion injury, traumatic brain injury, organ transplant, and neurodegeneration (Choadhry, J Thorac Cardiovasc Surg. 2007 July; 134(1):124-31; Mcllwain, Cold Spring Harb Perspect Biol 2013; 5:a008656). In addition, it is well known that increases in caspase activity indicates potential utility for treating diseases and disorders responsive to induction of apoptosis, including cancer, autoimmune disorders, rheumatoid arthritis, infectious diseases, inflammatory disease (Elmore, Toxicol Pathol. 2007; 35(4): 495-516). Changes in cell viability in cells treated with a test compounds are well known to be an indicator of potential therapeutic utility. A decrease in cell viability indicates potential utility for treating diseases and disorders responsive to changes in cell viability/proliferation, including for example cancer (Boyd, Drug Dev Res 34:91-109 (1995)). An increase in cell viability indicates potential utility for treating diseases associated with decreased cell viability, including diabetes, cardiovascular disease, ischemia reperfusion injury, traumatic brain injury, organ transplant, chemotherapy, and neurodegeneration. Additionally, an increase in cell viability indicates potential utility for improving cell viability of animal cells in culture.

Additionally, one skilled in the art can review structure-function studies identifying residues in similar peptides that are important for activity or structure. In view of such a comparison, the skilled artisan can predict the importance of amino acid residues in a peptide that correspond to amino acid residues important for activity or structure in similar peptides. One skilled in the art may opt for chemically similar amino acid substitutions for such predicted important amino acid residues.

One skilled in the art can also analyze the three-dimensional structure and amino acid sequence in relation to that structure in similar peptides. In view of such information, one skilled in the art may predict the alignment of amino acid residues of a peptide with respect to its three-dimensional structure. In certain embodiments, one skilled in the art may choose to not make radical changes to amino acid residues predicted to be on the surface of the peptide, since such residues may be involved in important interactions with other molecules. Moreover, one skilled in the art may generate test variants containing a single amino acid substitution at each desired amino acid residue. The variants can then be screened using activity assays known to those skilled in the art. Such variants could be used to gather information about suitable variants. For example, if one discovered that a change to a particular amino acid residue resulted in destroyed, undesirably reduced, or unsuitable activity, variants with such a change can be avoided. In other words, based on information gathered from such routine experiments, one skilled in the art can readily determine the amino acids where further substitutions should be avoided either alone or in combination with other mutations.

The term “derivative” as used herein means a chemically modified peptide, in which one or more side chains have been covalently attached to the peptide. The term “side chain” may also be referred to as a “substituent”. A derivative comprising such side chains will thus be “derivatized” peptide or “derivatized” analog. The term may also refer to peptides containing one or more chemical moieties not normally a part of the peptide molecule such as esters and amides of free carboxy groups, acyl and alkyl derivatives of free amino groups, phospho esters and ethers of free hydroxy groups. Such modifications may be introduced into the molecule by reacting targeted amino acid residues of the peptide with an organic derivatizing agent that is capable of reacting with selected side chains or terminal residues. Preferred chemical derivatives include peptides that have been phosphorylated, C-termini amidated or N-termini acetylated. The term may also refer to peptides as used herein which may be prepared from the functional groups which occur as side chains on the residues or the N- or C-terminal groups, by means known in the art, and are included herein as long as they remain pharmaceutically acceptable, i.e., they do not destroy the activity of the peptide, do not confer toxic properties on compositions containing it and do not adversely affect the antigenic properties thereof. These derivatives may, for example, include aliphatic esters of the carboxyl groups, amides of the carboxyl groups produced by reaction with ammonia or with primary or secondary amines, N-acyl derivatives of free amino groups of the amino acid residues formed by reaction with acyl moieties (e.g., alkanoyl or carbocyclic aroyl groups) or O-acyl derivatives of free hydroxyl group (for example that of seryl or threonyl residues) formed by reaction with acyl moieties.

A modified amino acid residue is an amino acid residue in which any group or bond was modified by deletion, addition, or replacement with a different group or bond, as long as the functionality of the amino acid residue is preserved or if functionality changed (for example replacement of tyrosine with substituted phenylalanine) as long as the modification did not impair the activity of the peptide containing the modified residue.

The term “substituent” or “side chain” as used herein means any suitable moiety bonded, in particular covalently bonded, to an amino acid residue, in particular to any available position on an amino acid residue. Typically, the suitable moiety is a chemical moiety.

The term “fatty acid” refers to aliphatic monocarboxylic acids having from 4 to 28 carbon atoms, it is preferably un-branched, and it may be saturated or unsaturated. In the present disclosure fatty acids comprising 10 to 16 amino acids are preferred.

The term “fatty diacid” refers to fatty acids as defined above but with an additional carboxylic acid group in the omega position. Thus, fatty diacids are dicarboxylic acids. In the present disclosure fatty acids comprising 14 to 20 amino acids are preferred.

The term “% sequence identity” is used interchangeably herein with the term “% identity” and refers to the level of amino acid sequence identity between two or more peptide sequences or the level of nucleotide sequence identity between two or more nucleotide sequences, when aligned using a sequence alignment program. For example, as used herein, 80% identity means the same thing as 80% sequence identity determined by a defined algorithm, and means that a given sequence is at least 80% identical to another length of another sequence.

The term “% sequence homology” is used interchangeably herein with the term “% homology” and refers to the level of amino acid sequence homology between two or more peptide sequences or the level of nucleotide sequence homology between two or more nucleotide sequences, when aligned using a sequence alignment program. For example, as used herein, 80% homology means the same thing as 80% sequence homology determined by a defined algorithm, and accordingly a homologue of a given sequence has greater than 80% sequence homology over a length of the given sequence.

Exemplary computer programs which can be used to determine degrees of identity or homology between two sequences include, but are not limited to, the suite of BLAST programs, e.g., BLASTN, BLASTX, and TBLASTX, BLASTP and TBLASTN, publicly available on the Internet at the NCBI website. See also Altschul et al., 1990, J. Mol. Biol. 215:403-10 (with special reference to the published default setting, i.e., parameters w=4, t=17) and Altschul et al., 1997, Nucleic Acids Res., 25:3389-3402. Sequence searches are typically carried out using the BLASTP program when evaluating a given amino acid sequence relative to amino acid sequences in the GenBank Protein Sequences and other public databases. The BLASTX program is preferred for searching nucleic acid sequences that have been translated in all reading frames against amino acid sequences in the GenBank Protein Sequences and other public databases. Both BLASTP and BLASTX are run using default parameters of an open gap penalty of 11.0, and an extended gap penalty of 1.0, and utilize the BLOSUM-62 matrix. (Id). In addition to calculating percent sequence identity, the BLAST algorithm also performs a statistical analysis of the similarity between two sequences (see, e.g., Karlin & Altschul, Proc. Nat'l. Acad. Sci. USA, 90:5873-5787 (1993)). One measure of similarity provided by the BLAST algorithm is the smallest sum probability (P(N)), which provides an indication of the probability by which a match between two nucleotide or amino acid sequences would occur by chance.

A “pharmaceutical composition” refers to a composition suitable for pharmaceutical use in an animal or human. A pharmaceutical composition comprises a pharmacologically and/or therapeutically effective amount of an active agent and a pharmaceutically acceptable excipient or carrier. Pharmaceutical compositions and methods for their preparation will be readily apparent to those skilled in the art. Such compositions and methods for their preparation may be found, for example, in Remington's Pharmaceutical Sciences, 19th Edition (Mack Publishing Company, 1995). The pharmaceutical compositions are generally formulated as sterile, substantially isotonic and in full compliance with all GMP regulations of the U.S. Food and Drug Administration. The term also encompasses any of the agents listed in the US Pharmacopeia for use in animals, including humans. Suitable pharmaceutical carriers and formulations are described in Remington's Pharmaceutical Sciences, 21st Ed. 2005, Mack Publishing Co, Easton.

“Pharmaceutically acceptable carrier” or “pharmaceutically acceptable excipient” refers to compositions that do not produce adverse, allergic, or other untoward reactions when administered to an animal or a human. As used herein, “pharmaceutically acceptable carrier” or “pharmaceutically acceptable excipient” includes any and all solvents, dispersion media, coatings, antibacterial and antifungal agents, isotonic and absorption delaying agents, and the like that are physiologically compatible. Some examples of pharmaceutically acceptable excipients are water, saline, phosphate buffered saline, dextrose, glycerol, ethanol and the like, as well as combinations thereof. In many cases, the excipients will include isotonic agents, for example, sugars, polyalcohols such as mannitol, sorbitol, or sodium chloride in the composition. Additional examples of pharmaceutically acceptable excipients are wetting agents or minor amounts of auxiliary substances such as wetting or emulsifying agents, preservatives or buffers, which enhance the shelf life or effectiveness of the peptide.

As used herein the term “pharmaceutically acceptable salt” refers to salts of peptides that retain the biological activity of the parent peptide, and which are not biologically or otherwise undesirable. Many of the peptides disclosed herein are capable of forming acid and/or base salts by virtue of the presence of amino and/or carboxyl groups or groups similar thereto. Pharmaceutically acceptable acid addition salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, and the like. Pharmaceutically acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines, such as ethanolamine, diethanolamine, triethanolamine, N-methylglucamine, dicyclohexylamine, and the like. Pharmaceutically acceptable salt also possesses the desired pharmacological activity of the parent compound.

It may be convenient or desirable to prepare, purify, and/or handle a corresponding solvate of the peptide. The term “solvate” is used herein in the conventional sense to refer to a complex of solute (e.g., peptide, salt of peptide) and solvent. If the solvent is water, the solvate may be conveniently referred to as a hydrate, for example, a mono-hydrate, a di-hydrate, a tri-hydrate, etc. Unless otherwise specified, a reference to a particular peptide also includes solvate and hydrate forms thereof.

The “co-crystal” or “co-crystal salt” as used herein means a crystalline material composed of two or more unique solids at room temperature, each of which has distinctive physical characteristics such as structure, melting point, and heats of fusion, hygroscopicity, solubility, and stability. A co-crystal or a co-crystal salt can be produced according to a per se known co-crystallization method. The terms co-crystal (or cocrystal) or co-crystal salt also refer to a multicomponent system in which there exists a host API (active pharmaceutical ingredient) molecule or molecules, such as a peptide of Formulas I-IV and II′-III′, and a guest (or co-former) molecule or molecules.

As used herein, a “therapeutically effective amount” of a peptide that when provided to a subject in accordance with the disclosed and claimed methods affects biological activities such as modulating cell signaling associated with aberrant cellular proliferation and malignancy, impacting cell viability and providing neuroprotection.

The terms “treat”, “treating” and “treatment” refer refers to an approach for obtaining beneficial or desired clinical results. Further, references herein to “treatment” include references to curative, palliative and/or prophylactic treatment. The term “treating” refers to inhibiting, preventing and/or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

The term “improving cell survival” refers to an increase in the number of cells that survive a given condition, as compared to a control, e.g., the number of cells that would survive the same conditions in the absence of treatment. Conditions can be in vitro, in vivo, ex vivo, or in situ. Improved cell survival can be expressed as a comparative value, e.g., twice as many cells survive if cell survival is improved two-fold. Improved cell survival can result from a reduction in apoptosis, an increase in the life-span of the cell, or an improvement of cellular function and condition.

For clarity, the term “instructing” is meant to include information on a label approved by a regulatory agency, in addition to its commonly understood definition.

The term “apelin receptor agonists” also includes those know in the art, as described in Conrad Fischer (2020) A patent review of apelin receptor (APJR) modulators (2014-2019), Expert Opinion on Therapeutic Patents, 30:4, 251-261, DOI: 10.1080/13543776.2020.1731473.

In an embodiment, the peptides may be administered as their nucleotide equivalents via gene therapy methods. The term “nucleotide equivalents” includes any nucleic acid which includes a nucleotide sequence that encodes a peptide. For example, the invention includes polynucleotides that comprise or consist of a nucleotide sequence that encodes a peptide described herein. The invention also includes vectors, including expression vectors, that comprise a nucleotide sequence that encodes a peptide described herein. Expression vectors include one or more expressing control sequences, such as a promoter, operably linked to the coding sequence such that the peptide is expressed in suitable host cells that contain the expression vector. In one embodiment, the peptide-related polynucleotide is encoded in a plasmid or vector, which may be derived from an adeno-associated virus (AAV). The AAV may be a recombinant AAV virus and may comprise a capsid serotype such as, but not limited to, of AAV1, AAV2, AAV3, AAV4, AAV5, AAV6, AAV7, AAV8, AAV9, AAV9.47, AAV9(hu14), AAV10, AAV11, AAV12, AAVrh8, AAVrh10, AAV-DJ, and AAV-DJ8. As a non-limiting example, the capsid of the recombinant AAV virus is AAV2. As a non-limiting example, the capsid of the recombinant AAV virus is AAVrh10. As a non-limiting example, the capsid of the recombinant AAV virus is AAV9(hu14). As a non-limiting example, the capsid of the recombinant AAV virus is AAV-DJ. As a non-limiting example, the capsid of the recombinant AAV virus is AAV9.47. As a non-limiting example, the capsid of the recombinant AAV virus is AAV-DJ8. An embodiment comprises the nucleotide equivalents of the peptide sequences of SEQ ID NO: 1-64 and 69-79.

A person skilled in the art may recognize that a target cell may require a specific promoter including but not limited to a promoter that is species specific, inducible, tissue-specific, or cell cycle-specific Parr et al, Nat. Med. 3:1145-9 (1997); the contents of which are herein incorporated by reference in its entirety).

As used herein, a “vector” is any molecule or moiety which transports, transduces or otherwise acts as a carrier of a heterologous molecule such as the polynucleotides of the invention. A “viral vector” is a vector which comprises one or more polynucleotide regions encoding or comprising payload molecule of interest, e.g., a transgene, a polynucleotide encoding a polypeptide or multi-polypeptide. Viral vectors of the present invention may be produced recombinantly and may be based on adeno-associated virus (AAV) parent or reference sequence. Serotypes which may be useful in the present invention include any of those arising from AAV1, AAV2, AAV3, AAV4, AAV5, AAV6, AAV7, AAV8, AAV9, AAV9.47, AAV9(hu14), AAV10, AAV11, AAV 12, AAVrh8, AAVrhIO, AAV-DJ, and AAV-DJ8.

In one embodiment, the serotype which may be useful in the present invention may be AAV-DJ8. The amino acid sequence of AAV-DJ8 may comprise two or more mutations in order to remove the heparin binding domain (HBD). As a non-limiting example, the AAV-DJ sequence described as SEQ ID NO: 1 in U.S. Pat. No. 7,588,772, the contents of which are herein incorporated by reference in its entirety, may comprise two mutations: (1) R587Q where arginine (R; arg) at amino acid 587 is changed to glutamine (Q; gln) and (2) R590T where arginine (R; arg) at amino acid 590 is changed to threonine (T; thr). As another non-limiting example, may comprise three mutations: (1) K406R where lysine (K; lys) at amino acid 406 is changed to arginine (R; arg), (2) R587Q where arginine (R; arg) at amino acid 587 is changed to glutamine (Q; gln) and (3) R590T where arginine (R; arg) at amino acid 590 is changed to threonine (T; thr).

AAV vectors may also comprise self-complementary AAV vectors (scAAVs). scAAV vectors contain both DNA strands which anneal together to form double stranded DNA. By skipping second strand synthesis, scAAVs allow for rapid expression in the cell.

In one embodiment, the pharmaceutical composition comprises a recombinant adeno-associated virus (AAV) vector comprising an AAV capsid and an AAV vector genome. The AAV vector genome may comprise at least one peptide related polynucleotide described herein, such as, but not limited to, SEQ ID NO: 1-64 and 69-79 or variants having at least 95% identity thereto. The recombinant AAV vectors in the pharmaceutical composition may have at least 70% which contain an AAV vector genome.

In one embodiment, the pharmaceutical composition comprises a recombinant adeno-associated virus (AAV) vector comprising an AAV capsid and an AAV vector genome. The AAV vector genome may comprise at least one peptide related polynucleotide described herein, such as, but not limited to, SEQ ID NO: 1-64 and 69-79 or variants having at least 95% identity thereto, plus an additional N-terminal proline. The recombinant AAV vectors in the pharmaceutical composition may have at least 70% which contain an AAV vector genome.

In one embodiment, the viral vector comprising a peptide-related polynucleotide may be administered or delivered using the methods for the delivery of AAV virions described in European Patent Application No. EP1857552, the contents of which are herein incorporated by reference in its entirety.

In one embodiment, the viral vector comprising a peptide-related polynucleotide may be administered or delivered using the methods for delivering proteins using AAV vectors described in European Patent Application No. EP2678433, the contents of which are herein incorporated by reference in its entirety.

In one embodiment, the viral vector comprising a peptide-related polynucleotide may be administered or delivered using the methods for delivering DNA molecules using AAV vectors described in U.S. Pat. No. 5,858,351, the contents of which are herein incorporated by reference in its entirety.

In one embodiment, the viral vector comprising a peptide-related polynucleotide may be administered or delivered using the methods for delivering DNA to the bloodstream described in U.S. Pat. No. 6,211,163, the contents of which are herein incorporated by reference in its entirety.

In one embodiment, the viral vector comprising a peptide-related polynucleotide may be administered or delivered using the methods for delivering AAV virions described in U.S. Pat. No. 6,325,998, the contents of which are herein incorporated by reference in its entirety.

In one embodiment, the viral vector comprising a peptide-related polynucleotide may be administered or delivered using the methods for delivering a payload to the central nervous system described in U.S. Pat. No. 7,588,757, the contents of which are herein incorporated by reference in its entirety.

In one embodiment, the viral vector comprising a peptide-related polynucleotide may be administered or delivered using the methods for delivering a payload described in U.S. Pat. No. 8,283,151, the contents of which are herein incorporated by reference in its entirety.

In one embodiment, the viral vector comprising a peptide-related polynucleotide may be administered or delivered using the methods for delivering a payload using a glutamic acid decarboxylase (GAD) delivery vector described in International Patent Publication No. WO 2001/089583, the contents of which are herein incorporated by reference in its entirety.

In one embodiment, the viral vector comprising a peptide-related polynucleotide may be administered or delivered using the methods for delivering a payload to neural cells described in International Patent Publication No. WO 2012/057363, the contents of which are herein incorporated by reference in its entirety.

In one embodiment, the viral vector comprising a peptide-related polynucleotide may be administered or delivered using the methods for delivering a payload to cells described in U.S. Pat. No. 9,585,971, the contents of which are herein incorporated by reference in its entirety.

In one embodiment, the viral vector comprising a peptide-related polynucleotide may be administered or delivered using the methods for delivering a payload to cells described in Deverman et al. Nature Biotechnology, 34, 204-09 (2016).

In one embodiment, the viral vector comprising a peptide-related polynucleotide may be administered or delivered using the methods for the delivery of AAV virions described in U.S. Pat. No. 7,198,951 [adeno-associated virus (AAV) serotype 9 sequences, vectors containing same, and uses therefor], U.S. Pat. No. 9,217,155 [isolation of novel AAV's and uses thereof], WO 2011/126808 [pharmacologically induced transgene ablation system], U.S. Pat. No. 6,015,709 [transcriptional activators, and compositions and uses related thereto], U.S. Pat. No. 7,094,604 [Production of pseudotyped recombinant AAV virions], WO 2016/126993 [anti-tau constructs], U.S. Pat. No. 7,094,604 [recombinant AAV capsid protein], U.S. Pat. No. 8,292,769 [Avian adenoassociated virus (aaav) and uses thereof], U.S. Pat. No. 9,102,949 [CNS targeting aav vectors and methods of use thereof], US 2016/0120960 [adeno-associated virus mediated gene transfer to the central nervous system], WO 2016/073693 [AADC polynucleotides for the treatment of Parkinson's disease], WO 2015/168666 [AAV VECTORS FOR RETINAL AND CNS GENE Therapy], US 2009/0117156 [Gene Therapy for Niemann-Pick Disease type A] or WO 2005/120581 [gene therapy for neurometabolic disorders].

The pharmaceutical compositions of viral vectors described herein may be characterized by one or more of bioavailability, therapeutic window and/or volume of distribution.

In some embodiments, peptide-related nucleotides and/or peptide-related nucleotide compositions of the present invention may be combined with, coated onto or embedded in a device. Devices may include, but are not limited to stents, pumps, and/or other implantable therapeutic device. Additionally, peptide-related nucleotides and/or peptide-related nucleotide compositions may be delivered to a subject while the subject is using a compression device such as, but not limited to, a compression device to reduce the chances of deep vein thrombosis (DVT) in a subject. The present invention provides for devices which may incorporate viral vectors that encode one or more peptide-related polynucleotide payload molecules. These devices contain in a stable formulation the viral vectors which may be immediately delivered to a subject in need thereof, such as a human patient.

Devices for administration may be employed to deliver the viral vectors comprising an peptide-related nucleotides of the present invention according to single, multi- or split-dosing regimens taught herein.

As used herein and in the appended claims, the singular forms “a,” “or,” and “the” include plural referents unless the context clearly dictates otherwise. It is understood that aspects and variations of the disclosure described herein include “consisting” and/or “consisting essentially of” aspects and variation.

The term “about” as used herein means greater or lesser than the value or range of values stated by 10%, but is not intended to designate any value or range of values to only this broader definition. Each value or range of values preceded by the term “about” is also intended to encompass the embodiment of the stated absolute value or range of values.

As used herein, the term “preventing” refers to keeping a disease, disorder or condition from occurring in a subject who may be at risk for the disease, but has not yet been diagnosed as having the disease.

As used herein, each of the terms “subject” and “patient” includes mammals, preferably human beings at any age which suffer from the pathology. In some variations or contexts, this term encompasses individuals who are at risk to develop the pathology.

The pharmaceutical compositions are typically suitable for parenteral administration. As used herein, “parenteral administration” of a pharmaceutical composition includes any route of administration characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous injection, intraperitoneal injection, intramuscular injection, intrasternal injection, intravenous injection, intraarterial injection, intrathecal injection, intraventricular injection, intraurethral injection, intracranial injection, intrasynovial injection or infusions; or kidney dialytic infusion techniques.

In various embodiments, the peptide is admixed with a pharmaceutically acceptable excipients to form a pharmaceutical composition that can be systemically administered to the subject orally or via intravenous injection, intramuscular injection, subcutaneous injection, intraperitoneal injection, transdermal injection, intra-arterial injection, intrasternal injection, intrathecal injection, intraventricular injection, intraurethral injection, intracranial injection, intrasynovial injection or via infusions. The pharmaceutical composition preferably contains at least one component that is not found in nature.

Formulations of a pharmaceutical composition suitable for parenteral administration typically generally comprise the active ingredient combined with a pharmaceutically acceptable excipient, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for parenteral administration include, but are not limited to, suspensions, solutions, emulsions in oily or aqueous vehicles, pastes, and the like. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition. Parenteral formulations also include aqueous solutions which may contain carriers such as salts, carbohydrates and buffering agents (preferably to a pH of from 3 to 9), but, for some applications, they may be more suitably formulated as a sterile nonaqueous solution or as a dried form to be used in conjunction with a suitable vehicle such as sterile, pyrogen-free water. Exemplary parenteral administration forms include solutions or suspensions in sterile aqueous solutions, for example, aqueous propylene glycol or dextrose solutions. Such dosage forms can be suitably buffered, if desired. Other parentally-administrable formulations which are useful include those which comprise the active ingredient in microcrystalline form, or in a liposomal preparation. Formulations for parenteral administration may be formulated to be immediate and/or modified release. Modified release formulations include delayed-, sustained-, pulsed-, controlled-, targeted and programmed release.

The present disclosure includes compositions and methods for transdermal or topical delivery, to act locally at the point of application, or to act systemically once entering the body's blood circulation. In these systems, delivery may be achieved by techniques such as direct topical application of a substance or drug in the form of an ointment or the like, or by adhesion of a patch with a reservoir or the like that holds the drug (or other substance) and releases it to the skin in a time-controlled fashion. For topical administration, the compositions can be in the form of emulsions, lotions, gels, creams, jellies, solutions, suspensions, ointments, and transdermal patches. Some topical delivery compositions may contain polyenylphosphatidylcholine (herein abbreviated “PPC”). In some cases, PRO can be used to enhance epidermal penetration. The term “polyenylphosphatidylcholine,” as used herein, means any phosphatidylcholine bearing two fatty acid moieties, wherein at least one of the two fatty acids is an unsaturated fatty acid with at least two double bonds in its structure, such as linoleic acid. Such topical formulations may comprise one or more emulsifiers, one or more surfactants, one or more polyglycols, one or more lecithins, one or more fatty acid esters, or one or more transdermal penetration enhancers. Preparations can include sterile aqueous or nonaqueous solutions, suspensions and emulsions, which can be isotonic with the blood of the subject in certain embodiments. Examples of nonaqueous solvents are polypropylene glycol, polyethylene glycol, vegetable oil such as olive oil, sesame oil, coconut oil, arachis oil, peanut oil, mineral oil, organic esters such as ethyl oleate, or fixed oils including synthetic mono or di-glycerides. Aqueous solvents include water, alcoholic/aqueous solutions, emulsions or suspensions, including saline and buffered media. Parenteral vehicles include sodium chloride solution, 1,3-butandiol, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, antioxidants, chelating agents and inert gases and the like.

For example, in one aspect, sterile injectable solutions can be prepared by incorporating a peptide in the required amount in an appropriate solvent with one or a combination of ingredients enumerated above, as required, followed by filtered sterilization. Generally, dispersions are prepared by incorporating the active peptide into a sterile vehicle that contains a basic dispersion medium and the required other ingredients from those enumerated above. In the case of sterile powders for the preparation of sterile injectable solutions, methods of preparation such as vacuum drying and freeze-drying yield a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin. In various embodiments, the injectable compositions will be administered using commercially available disposable injectable devices.

The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind known in the art. Injectable formulations are in accordance with the disclosure. The requirements for effective pharmaceutical excipients for injectable compositions are well-known to those of ordinary skill in the art (see, e.g., Pharmaceutics and Pharmacy Practice, J. B. Lippincott Company, Philadelphia, Pa., Banker and Chalmers, eds., pages 238-250 (1982), and ASHP Handbook on Injectable Drugs, Toissel, 4th ed., pages 622-630 (1986)).

Additionally, the peptides of the present disclosures can be made into suppositories for rectal administration by mixing with a variety of bases, such as emulsifying bases or water-soluble bases. Formulations suitable for vaginal administration can be presented as pessaries, tampons, creams, gels, pastes, foams, or spray formulas containing, in addition to the active ingredient, such carriers as are known in the art to be appropriate.

It will be appreciated by one of skill in the art that, in addition to the above-described pharmaceutical compositions, the peptides of the disclosure can be formulated as inclusion complexes, such as cyclodextrin inclusion complexes, or liposomes.

The peptide can be administered intranasally or by inhalation, typically in the form of a dry powder (either alone, as a mixture, or as a mixed component particle, for example, mixed with a suitable pharmaceutically acceptable carrier) from a dry powder inhaler, as an aerosol spray from a pressurized container, pump, spray, atomiser (preferably an atomiser using electrohydrodynamics to produce a fine mist), or nebulizer, with or without the use of a suitable propellant, or as nasal drops. The pressurized container, pump, spray, atomizer, or nebulizer generally contains a solution or suspension of a peptide comprising, for example, a suitable agent for dispersing, solubilizing, or extending release of the active, a propellant(s) as solvent. Prior to use in a dry powder or suspension formulation, the drug product is generally micronized to a size suitable for delivery by inhalation (typically less than 5 microns). This may be achieved by any appropriate comminuting method, such as spiral jet milling, fluid bed jet milling, supercritical fluid processing to form nanoparticles, high pressure homogenization, or spray drying. Capsules, blisters and cartridges for use in an inhaler or insufflator may be formulated to contain a powder mix of the peptide, a suitable powder base and a performance modifier. Suitable flavors, such as menthol and levomenthol, or sweeteners, such as saccharin or saccharin sodium, may be added to those formulations intended for inhaled/intranasal administration. Formulations for inhaled/intranasal administration may be formulated to be immediate and/or modified release. Modified release formulations include delayed-, sustained-, pulsed-, controlled-, targeted and programmed release. In the case of dry powder inhalers and aerosols, the dosage unit is determined by means of a valve which delivers a metered amount. Units are typically arranged to administer a metered dose or “puff” of a peptide. The overall daily dose will typically be administered in a single dose or, more usually, as divided doses throughout the day.

According to one aspect, the peptides are for use in medicine, particularly human medicine. The peptides are effective to modulate cell signaling associated with apelin pathway. Additionally, the disclosure provides peptides effective in impacting lung disease.

In another aspect, there is provided a peptide, for use in in the prevention and/or treatment of infectious diseases, and lung diseases.

Acute respiratory distress syndrome (ARDS, the most severe form of acute lung injury, is a devastating clinical syndrome with high mortality rate (30-60%). Predisposing factors for ARDS are diverse and include sepsis, aspiration, and pneumonias including infections with coronavirus.

Generally, all patients with acute lung disorders (specifically all acute lung disorders which need intensive treatment, such as the ones described above or others, such as ARDS in general, Pneumonia-induced or Anthrax-induced acute lung injuries) which require treatment in the intensive health care unit of a hospital can benefit from administration of apelin mimetics according to the present invention.

### Combination Therapy

According to another embodiment, the peptides are co-administered or co-formulated with other known therapeutic agents. According to a further aspect of the present disclosure, provided herein is a combination treatment comprising the administration of a pharmacologically effective amount of a peptide or peptide analog according to the present disclosure, or a pharmaceutically acceptable salt thereof, optionally together with a pharmaceutically acceptable diluent or carrier, with the simultaneous, sequential or separate administration of one or more antivirals, agents for treating coronavirus-related symptoms, or agents for treating lung injury.

“Agents for treating coronavirus-related symptoms” include vitamin C, nucleotide analogs, protease inhibitors, membrane fusion inhibitors, antimalarials, ACE inhibitors, ACE2 inhibitors, anti-ACE2 antibodies, recombinant ACE2, anti-MASP-2 antibodies, anti-C5-antibodies, immunomodulators, IL-1 inhibitors and IL-6 inhibitors. Nucleotide analogs include remdesivir. Protease inhibitors include HIV protease inhibitors such as lopinavir, ritonavir, indinavir, atazanavir, boceprevir, darunavir, fosamprenavir, nelfinavir, saquinavir, simeprevir, telaprevir and tipranavir. Membrane fusion inhibitors include umifenovir, Examples of antimalarials are chloroquine and hydroxychloroquine. Immunomodulators include interferon beta 1a. ACE inhibitors include benazepril, captopril, enalapril, fosinopril, lisinopril, moexipril, perindopril, quinapril, ramipril, and trandolapril.

Without being bound by a specific theory, agonists of apelin receptor play a role in acute lung disease. Increases in fluid accumulation occur with viral infections, such as coronaviral infections and COVID-19 as well as influenza. Peptides that have an effect on the apelin receptor, especially those with a better stability than apelin or other naturally occurring peptides, have potential utility for treatment of acute lung disease, especially those as a result of infections.

The person skilled in the art can easily determine whether the peptide is biologically active. For example, the capacity to activate the apelin/apelin receptor pathway can be determined by assessing inhibition of cAMP production induced by forskolin, ERK phosphorylation and towards apelin receptor internalization (e.g. as described in Example). Agonistic activities of an apelin analogue toward APJ may be determined by any well-known method in the art. For example, since the compound of the present invention can promote the function of the apelin receptor, the agonist can be screened by using apelin, the natural agonist of APJ in a competitive binding test and test associated with the biological activity.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a subject may also be determined, as can the temporal requirements for administering each agent to provide a detectable therapeutic benefit to the subject. Accordingly, while certain dose and administration regimens are exemplified herein, these examples in no way limit the dose and administration regimen that may be provided to a subject in practicing the present disclosure.

It is to be noted that dosage values may vary with the type and severity of the condition to be ameliorated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. Further, the dosage regimen with the compositions of this disclosure may be based on a variety of factors, including the type of disease, the age, weight, sex, medical condition of the subject, the severity of the condition, the route of administration, and the particular peptide employed. Thus, the dosage regimen can vary widely, but can be determined routinely using standard methods. For example, doses may be adjusted based on pharmacokinetic or pharmacodynamic parameters, which may include clinical effects such as toxic effects and/or laboratory values. Thus, the present disclosure encompasses intra-subject dose-escalation as determined by the skilled artisan. Determining appropriate dosages and regimens are well-known in the relevant art and would be understood to be encompassed by the skilled artisan once provided the teachings disclosed herein.

The dose of the peptide of the present disclosure also will be determined by the existence, nature and extent of any adverse side effects that might accompany the administration of a particular peptide of the present disclosure. Typically, the attending physician will decide the dosage of the peptide of the present disclosure with which to treat each individual patient, taking into consideration a variety of factors, such as age, body weight, general health, diet, sex, peptide of the present disclosure to be administered, route of administration, and the severity of the condition being treated. By way of example and not intending to be limiting, the dose of the peptide of the present disclosure can be about 0.0001 to about 100 mg/kg body weight of the subject being treated/day, from about 0.001 to about 10 mg/kg body weight/day, or about 0.01 mg to about 1 mg/kg body weight/day. The peptide can be administered in one or more doses, such as from 1 to 3 doses.

In some embodiments, the pharmaceutical composition comprises any of the analogs disclosed herein at a purity level suitable for administration to a patient. In some embodiments, the analog has a purity level of at least about 90%, preferably above about 95%, more preferably above about 99%, and a pharmaceutically acceptable diluent, carrier or excipient.

The pharmaceutical compositions may be formulated to achieve a physiologically compatible pH. In some embodiments, the pH of the pharmaceutical composition may be at least 5, or at least 6, or at least 7, depending on the formulation and route of administration.

In various embodiments, single or multiple administrations of the pharmaceutical compositions are administered depending on the dosage and frequency as required and tolerated by the subject. In any event, the composition should provide a sufficient quantity of at least one of the peptide disclosed herein to effectively treat the subject. The dosage can be administered once but may be applied periodically until either a therapeutic result is achieved or until side effects warrant discontinuation of therapy.

The dosing frequency of the administration of the peptide pharmaceutical composition depends on the nature of the therapy and the particular disease being treated. The administration may be once, twice, three times or four times daily, for the peptide. Treatment of a subject with a therapeutically effective amount of a peptide, can include a single treatment or, preferably, can include a series of treatments, in a preferred example, a subject is treated with peptide daily, one time per week or biweekly.

Reference will now be made in detail to embodiments of the present disclosure. While certain embodiments of the present disclosure will be described, it will be understood that it is not intended to limit the embodiments of the present disclosure to those described embodiments. To the contrary, reference to embodiments of the present disclosure is intended to cover alternatives, modifications, and equivalents as may be included within the spirit and scope of the embodiments of the present disclosure as defined by the appended claims.

### Embodiments

The embodiments listed below are presented in numbered form for convenience and for ease and clarity of reference in referring back to multiple embodiments.

1. A method of treating a coronavirus infection in a subject, comprising administering to the subject a peptide comprising an amino acid sequence of Formula I:

(I)
(SEQ ID NO: 1)
X¹-RX²-X³-X⁴-X⁵-X⁶-Q-X⁷-L-X⁸-X⁹

wherein X¹ is absent or if present is an amino acid having a polar side chain or a non-polar side chain; X² is an amino acid having a polar side chain or a non-polar side chain; X³ is absent or if present is one to three amino acids, each amino acid independently having a polar side chain or a non-polar side chain; X⁴ is an amino acid having a polar side chain or a non-polar side chain; X⁵ is an amino acid having a non-polar side chain; X⁶ is an amino acid having a polar side chain or a non-polar side chain; X⁷ is an amino acid having a polar side chain; X⁸ is an amino acid having a polar side chain; and X⁹ is absent or if present is one to three amino acids, each amino acid independently having a polar side chain or a non-polar side chain;

or administering an analog of said peptide having a deletion, insertion or substitution of one, two, three, or four amino acids; or administering a C-terminal acid or amide, or N-acetyl derivative thereof; or administering a pegylated derivative thereof; or administering a pharmaceutically acceptable salt thereof.

2. A peptide, peptide analog, or derivative thereof, or a pharmaceutically acceptable salt thereof for use in the treatment of a subject having or suspected of having a coronavirus infection, the peptide comprising an amino acid sequence of Formula I:

(I)
(SEQ ID NO: 1)
X¹-RX²-X³-X⁴-X⁵-X⁶-Q-X⁷-L-X⁸-X⁹

wherein X¹ is absent or if present is an amino acid having a polar side chain or a non-polar side chain; X² is an amino acid having a polar side chain or a non-polar side chain; X³ is absent or if present is one to three amino acids, each amino acid independently having a polar side chain or a non-polar side chain; X⁴ is an amino acid having a polar side chain or a non-polar side chain; X⁵ is an amino acid having a non-polar side chain; X⁶ is an amino acid having a polar side chain or a non-polar side chain; X⁷ is an amino acid having a polar side chain; X⁸ is an amino acid having a polar side chain; and X⁹ is absent or if present is one to three amino acids, each amino acid independently having a polar side chain or a non-polar side chain; the analog of said peptide having a deletion, insertion or substitution of one, two, three, or four amino acids; the derivative comprising a C-terminal acid or amide, or a N-acetyl derivative, or a pegylated derivative.

3. A method of treating a subject in need of treatment for sepsis, septic shock, ischemic shock, or organ failure associated with a viral infection, the method comprising administering to the subject a peptide comprising an amino acid sequence of Formula I:

(I)
(SEQ ID NO: 1)
X¹-RX²-X³-X⁴-X⁵-X⁶-Q-X⁷-L-X⁸-X⁹

wherein X¹ is absent or if present is an amino acid having a polar side chain or a non-polar side chain; X² is an amino acid having a polar side chain or a non-polar side chain; X³ is absent or if present is one to three amino acids, each amino acid independently having a polar side chain or a non-polar side chain; X⁴ is an amino acid having a polar side chain or a non-polar side chain; X⁵ is an amino acid having a non-polar side chain; X⁶ is an amino acid having a polar side chain or a non-polar side chain; X⁷ is an amino acid having a polar side chain; X⁸ is an amino acid having a polar side chain; and X⁹ is absent or if present is one to three amino acids, each amino acid independently having a polar side chain or a non-polar side chain;

or administering an analog of said peptide having a deletion, insertion or substitution of one, two, three, or four amino acids; or administering a derivative comprising a C-terminal acid or amide, or a N-acetyl derivative thereof, or a pegylated derivative thereof; or administering a pharmaceutically acceptable salt thereof.

4. A peptide, peptide analog, or derivative thereof, or a pharmaceutically acceptable salt thereof for use in the treatment of sepsis, septic shock, ischemic shock, or organ failure associated with a viral infection, the peptide comprising an amino acid sequence of Formula I:

(I)
(SEQ ID NO: 1)
X¹-RX²-X³-X⁴-X⁵-X⁶-Q-X⁷-L-X⁸-X⁹

wherein X¹ is absent or if present is an amino acid having a polar side chain or a non-polar side chain; X² is an amino acid having a polar side chain or a non-polar side chain; X³ is absent or if present is one to three amino acids, each amino acid independently having a polar side chain or a non-polar side chain; X⁴ is an amino acid having a polar side chain or a non-polar side chain; X⁵ is an amino acid having a non-polar side chain; X⁶ is an amino acid having a polar side chain or a non-polar side chain; X⁷ is an amino acid having a polar side chain; X⁸ is an amino acid having a polar side chain; and X⁹ is absent or if present is one to three amino acids, each amino acid independently having a polar side chain or a non-polar side chain; the analog of said peptide having a deletion, insertion or substitution of one, two, three, or four amino acids; the derivative comprising a C-terminal acid or amide, or a N-acetyl derivative thereof, or a pegylated derivative thereof.

5. The method or the peptide, analog, derivative, or salt for use of any one of embodiments 1 to 4, wherein X¹ is M, K, or absent; X² is R or Aib; X³ is absent or is M, E, -MMG-, -LLG-, -II(dA)-, -Nle-Nle-G- or -IIG-; X⁴ is M, E, L, I or Nle; X⁵ is V, A or G; X⁶ is F, Y, A or E; X⁷ is C, S or E; X⁸ is C, S or E; and X⁹ is -GL, -G(dA), -G(dA)K, -(dA)L, G or absent.

6. The method or the peptide, analog, derivative, or salt for use of any one of embodiments 1 to 4, wherein, in the peptide or derivative, X¹ is (PEG12)-K, and/or wherein X⁹ is -G(dA)-K(PEG12).

7. The method or the peptide, analog, derivative, or salt for use of any one of embodiments 1 to 4, wherein X³ is absent or is -LLG-; X⁴ is L; X⁵ is V; or X⁸ is C or E.

8. The method or the peptide, analog, derivative, or salt for use of any one of embodiments 1 to 4, wherein X⁷ is S.

9. The method or the peptide, analog, derivative, or salt for use of any one of embodiments 1 to 4, wherein the peptide or peptide derivative comprises or consists of an amino acid sequence selected from SEQ ID NOs: 2-63.

10. The method or the peptide, analog, derivative, or salt for use of any one of embodiments 1 to 4, wherein the peptide or peptide derivative comprises or consists of an amino acid sequence selected from MRRMMGMVFQCLCGL (SEQ ID NO: 7); RRMMGMVFQCLCG(dA) (SEQ ID NO: 8); RRMMGMVYQCLCG(dA) (SEQ ID NO: 10); RRMMGMVAQCLCG(dA) (SEQ ID NO: 11); RRMMGMVFQELCG(dA) (SEQ ID NO: 13); RRMMGMVFQCLEG(dA) (SEQ ID NO: 14); RRMMGMVFQSLCG(dA) (SEQ ID NO: 15); RR(Nle)(Nle)G(Nle)VFQCLCG(dA) (SEQ ID NO: 18); (PEG12)KRRMMGMVFQCLCG(dA) (SEQ ID NO: 20); RRMMGMVFQCLCG(dA)K(PEG12) (SEQ ID NO: 21); RRMVYQCLCG(dA) (SEQ ID NO: 22); RRMMGMVAQCLEG(dA) (SEQ ID NO: 30); R(Aib)MMGMVFQSLCG(dA) (SEQ ID NO: 34); (PEG12)KRRMMGMVFQSLCG(dA) (SEQ ID NO: 36); (PEG12)KRRLLGLVFQSLCG(dA) (SEQ ID NO: 37); (PEG12)KRRIIGIVFQCLCG(dA) (SEQ ID NO: 42); RRIIGIVFQSLCG(dA) (SEQ ID NO: 43).

11. A method of treating a coronavirus infection in a subject in need thereof, comprising administering to the subject a peptide comprising an amino acid sequence of Formula III′:

(III′)
(SEQ ID NO: 78)
X¹⁸-R-X¹⁹-X²⁰-X²¹ V-X²²-Q-X²³ L-X²⁴-G-X²⁵

wherein X¹⁸ is absent or if present is M or K; X¹⁹ is R or Aib; X²⁰ is absent or if present is -M-M-G- or Nle-Nle-G-; X²¹ is M or Nle; X²² is F, A or Y; X²³ is S, E or C; X²⁴ is E or C; X²⁵ is L, dA or -dA-K; or administering a C-terminal acid or amide thereof, or a N-acetyl derivative thereof; or administering a pegylated derivative thereof; or administering a pharmaceutically acceptable salt thereof.

12. A peptide, derivative thereof, or a pharmaceutically acceptable salt thereof for use in the treatment of a subject having or suspected of having a coronavirus infection, the peptide comprising an amino acid sequence of Formula III′:

(III′)
(SEQ ID NO: 78)
X¹⁸-R-X¹⁹-X²⁰-X²¹ V-X²²-Q-X²³ L-X²⁴-G-X²⁵

wherein X¹⁸ is absent or if present is M or K; X¹⁹ is R or Aib; X²⁰ is absent or if present is -M-M-G- or Nle-Nle-G-; X²¹ is M or Nle; X²² is F, A or Y; X²³ is S, E or C; X²⁴ is E or C; X²⁵ is L, dA or -dA-K; the derivative comprising a C-terminal acid or amide, or N-acetyl derivative thereof; or a pegylated derivative thereof.

13. A method of treating a subject in need of treatment for sepsis, septic shock, ischemic shock, or organ failure associated with a viral infection, the method comprising administering to the subject a peptide comprising an amino acid sequence of Formula III′:

(III′)
(SEQ ID NO: 78)
X¹⁸-R-X¹⁹-X²⁰-X²¹ V-X²²-Q-X²³ L-X²⁴-G-X²⁵

wherein X¹⁸ is absent or if present is M or K; X¹⁹ is R or Aib; X²⁰ is absent or if present is -M-M-G- or Nle-Nle-G-; X²¹ is M or Nle; X²² is F, A or Y; X²³ is S, E or C; X²⁴ is E or C; X²⁵ is L, dA or -dA-K; or administering a derivative thereof, the derivative comprising a C-terminal acid or amide, or N-acetyl derivative thereof; or a pegylated derivative thereof; or administering a pharmaceutically acceptable salt thereof.

14. A peptide, derivative thereof, or a pharmaceutically acceptable salt thereof for use in the treatment of sepsis, septic shock, ischemic shock, or organ failure associated with a viral infection, the peptide comprising an amino acid sequence of Formula III′:

| | (III′) |
|---|---|
| (SEQ ID NO: 78) | |
| | X¹⁸-R-X¹⁹-X²⁰-X²¹ V-X²²-Q-X²³ L-X²⁴-G-X²⁵ |

wherein X¹⁸ is absent or if present is M or K; X¹⁹ is R or Aib; X²⁰ is absent or if present is -M-M-G- or Nle-Nle-G-; X²¹ is M or Nle; X²² is F, A or Y; X²³ is S, E or C; X²⁴ is E or C; X²⁵ is L, dA or -dA-K; the derivative comprising a C-terminal acid or amide, or N-acetyl derivative thereof; or a pegylated derivative thereof.

15. The method or the peptide, derivative, or salt for use of any one of embodiments 11-14, wherein X²⁵ is dA.

16. The method or the peptide, derivative, or salt for use of any one of embodiments 11-14, wherein X¹⁹ is R; X²⁰ is absent or is -M-M-G-; and X²¹ is M.

17. The method or the peptide, derivative, or salt for use of any one of embodiments 11-14, wherein X²² is F; and X²³ is C.

18. The method or the peptide, derivative, or salt for use of any one of embodiments 11-14, wherein the peptide or derivative comprises or consists of an amino acid sequence selected from MRRMMGMVFQCLCGL (SEQ ID NO: 7); RRMMGMVFQSLCG(dA) (SEQ ID NO: 15); and (PEG12)KRRMMGMVFQSLCG(dA) (SEQ ID NO: 36).

19. The method or the peptide, derivative, or salt for use of any one of embodiments 11-14, wherein the peptide or derivative comprises or consists of an amino acid sequence selected from (PEG12)RRMMGMVFQSLCG(dA) (SEQ ID NO: 71); and (K(PEG12))RRMMGMVFQSLCG(dA) (SEQ ID NO: 72).

20. A method of treating a coronavirus infection in a subject in need thereof, comprising administering to the subject a peptide comprising an amino acid sequence of Formula IV:

| | (IV) |
|---|---|
| (SEQ ID NO: 70) | |
| | X²⁶-RR-X²⁷-X²⁸ G-X²⁹-VFQ-X³⁰-LCG-(dA) |

wherein X²⁶ is absent or if present is K; X²⁷ is L or I; X²⁸ is L or I; X²⁹ is L or I; and X³⁰ is S or C;

or administering a C-terminal acid or amide, or N-acetyl derivative thereof; or administering a pegylated derivative thereof; or administering a pharmaceutically acceptable salt thereof.

21. A peptide, derivative thereof, or a pharmaceutically acceptable salt thereof for use in the treatment of a subject having or suspected of having a coronavirus infection, the peptide comprising an amino acid sequence of Formula IV:

| | (IV) |
|---|---|
| (SEQ ID NO: 70) | |
| | X²⁶-RR-X²⁷-X²⁸ G-X²⁹-VFQ-X³⁰-LCG-(dA) |

wherein X²⁶ is absent or if present is K; X²⁷ is L or I; X²⁸ is L or I; X²⁹ is L or I; and X³⁰ is S or C; the derivative comprising a C-terminal acid or amide, or a N-acetyl derivative thereof; or a pegylated derivative thereof.

22. A method of treating a subject in need of treatment for sepsis, septic shock, ischemic shock, or organ failure associated with a viral infection, the method comprising administering to the subject a peptide comprising an amino acid sequence of Formula IV:

| | (IV) |
|---|---|
| (SEQ ID NO: 70) | |
| | X²⁶-RR-X²⁷-X²⁸ G-X²⁹-VFQ-X³⁰-LCG-(dA) |

wherein X²⁶ is absent or if present is K; X²⁷ is L or I; X²⁸ is L or I; X²⁹ is L or I; and X³⁰ is S or C; or administering a derivative thereof, the derivative comprising a C-terminal acid or amide, or N-acetyl derivatives thereof; or a pegylated derivative thereof; or administering a pharmaceutically acceptable salt thereof.

23. A peptide, or derivative thereof, or pharmaceutically acceptable salt thereof, for use in the treatment of sepsis, septic shock, ischemic shock, or organ failure associated with a viral infection, the peptide comprising an amino acid sequence of Formula IV:

| | (IV) |
|---|---|
| (SEQ ID NO: 70) | |
| | X²⁶-RR-X²⁷-X²⁸ G-X²⁹-VFQ-X³⁰-LCG-(dA) |

wherein X²⁶ is absent or if present is K; X²⁷ is L or I; X²⁸ is L or I; X²⁹ is L or I; and X³⁰ is S or C; the derivative comprising a C-terminal acid or amide, or a N-acetyl derivative thereof; or a pegylated derivative thereof.

24. The method or the peptide, derivative, or salt for use of any one of embodiments 20 to 23, wherein X³⁰ is S.

25. The method or the peptide, derivative, or salt for use of any one of embodiments 20 to 23, wherein X²⁷ is L; X²⁸ is L; and/or X²⁹ is L.

26. The method or the peptide, derivative, or salt for use of any one of embodiments 20 to 23, wherein the peptide or the derivative comprises or consists of an amino acid sequence selected from (PEG12)KRRLLGLVFQSLCG(dA) (SEQ ID NO: 37); or RRIIGIVFQSLCG(dA) (SEQ ID NO: 43).

27. The method or the peptide, derivative, or salt for use of any one of embodiments 20 to 23, wherein the peptide or the derivative comprises or consists of an amino acid sequence selected from (K(PEG12))RRLLGLVFQSLCG(dA) (SEQ ID NO: 73); (PEG12)RRLLGLVFQSLCG(dA) (SEQ ID NO: 74); (PEG12)KRRIIGIVFQSLCG(dA) (SEQ ID NO: 75); (K(PEG12))RRIIGIVFQSLCG(dA) (SEQ ID NO: 76); and (PEG12}RRIIGIVFQSLCG(dA) (SEQ ID NO: 77).

28. The method or the peptide, analog, derivative, or salt for use of any one of embodiments 1-2, 5-12, 15-21, and 24-27, wherein the coronavirus infection is SARS or COVID-19 infection.

29. The method or the peptide, analog, derivative, or salt for use of any one of embodiments 1-2, 5-12, 15-21, and 24-27, wherein the coronavirus infection causes acute lung injury or acute respiratory distress syndrome.

30. The method or the peptide, analog, derivative, or salt for use of any one of embodiments 1-2, 5-12, 15-21, and 24-27, wherein the coronavirus infection potentiates bacteria-induced acute lung damage.

31. The method or the peptide, analog, derivative, or salt for use of any one of embodiments 1-2, 5-12, 15-21, and 24-27, wherein the peptide is administered together with an agent for treating coronavirus-related symptoms.

32. A method of modulating pro-inflammatory cytokine secretion comprising administering to the subject a peptide comprising an amino acid sequence of Formula III′:

| | (III′) |
|---|---|
| (SEQ ID NO: 78) | |
| | X¹⁸-R-X¹⁹-X²⁰-X²¹ V-X²²-Q-X²³ L-X²⁴-G-X²⁵ |

wherein X¹⁸ is absent or if present is M or K; X¹⁹ is R or Aib; X²⁰ is absent or if present is -M-M-G- or Nle-Nle-G-; X²¹ is M or Nle; X²² is F, A or Y; X²³ is S, E or C; X²⁴ is E or C; X²⁵ is L, dA or -dA-K; or administering a C-terminal acid or amide, or a N-acetyl derivative thereof; or administering a pegylated derivative thereof; or administering a pharmaceutically acceptable salt thereof.

33. A peptide, or derivative thereof, or pharmaceutically acceptable salt thereof, for use in the treatment of a subject having or suspected of having pro-inflammatory cytokine secretion, the peptide comprising an amino acid sequence of Formula III′:

| | (III′) |
|---|---|
| (SEQ ID NO: 78) | |
| | X¹⁸-R-X¹⁹-X²⁰-X²¹ V-X²²-Q-X²³ L-X²⁴-G-X²⁵ |

wherein X¹⁸ is absent or if present is M or K; X¹⁹ is R or Aib; X²⁰ is absent or if present is -M-M-G- or Nle-Nle-G-; X²¹ is M or Nle; X²² is F, A or Y; X²³ is S, E or C; X²⁴ is E or C; X²⁵ is L, dA or -dA-K; the derivative comprising a C-terminal acid or amide, or N-acetyl derivative thereof; or a pegylated derivative thereof.

34. The method or the peptide, derivative, or salt for use of embodiment 32 or 33, wherein X²⁵ is dAf.

35. The method or the peptide, derivative, or salt for use of embodiment 32 or 33, wherein X¹⁹ is R; X²⁰ is absent or is -M-M-G-; and X²¹ is M.

36. The method or the peptide, derivative, or salt for use of embodiment 32 or 33, wherein X²² is F; and X²³ is C.

37. The method or the peptide, derivative, or salt for use of embodiment 32 or 33, wherein the peptide or derivative comprises or consists of an amino acid sequence selected from MRRMMGMVFQCLCGL (SEQ ID NO: 7); RRMMGMVFQSLCG(dA) (SEQ ID NO: 15); and (PEG12)KRRMMGMVFQSLCG(dA) (SEQ ID NO: 36).

38. The method or the peptide, derivative, or salt for use of embodiment 32 or 33, wherein the peptide or derivative comprises or consists of an amino acid sequence selected from (PEG12)RRMMGMVFQSLCG(dA) (SEQ ID NO: 71); and (K(PEG12))RRMMGMVFQSLCG(dA) (SEQ ID NO: 72).

39. A method of modulating pro-inflammatory cytokine secretion comprising administering to the subject a peptide comprising either an amino acid sequence of Formula IV:

| | (IV) |
|---|---|
| (SEQ ID NO: 70) | |
| | X²⁶-RR-X²⁷-X²⁸ G-X²⁹-VFQ-X³⁰-LCG-(dA) |

wherein X²⁶ is absent or if present is K; X²⁷ is L or I; X²⁸ is L or I; X²⁹ is L or I; and X³⁰ is S or C; or administering a C-terminal acid or amide, or a N-acetyl derivative thereof; or administering a pegylated derivative thereof; or administering a pharmaceutically acceptable salt thereof.

40. A peptide, or derivative thereof, or pharmaceutically acceptable salt thereof for use in the treatment of a subject having or suspected of having pro-inflammatory cytokine secretion, the peptide comprising an amino acid sequence of Formula IV:

| | (IV) |
|---|---|
| (SEQ ID NO: 70) | |
| | X²⁶-RR-X²⁷-X²⁸ G-X²⁹-VFQ-X³⁰-LCG-(dA) |

wherein X²⁶ is absent or if present is K; X²⁷ is L or I; X²⁸ is L or I; X²⁹ is L or I; and X³⁰ is S or C; the derivative comprising a C-terminal acid or amide, or N-acetyl derivative thereof; or a pegylated derivative thereof.

41. The method or the peptide, derivative, or salt for use of embodiment 39 or 40, wherein X³⁰ is S.

42. The method or the peptide, derivative, or salt for use of embodiment 39 or 40, wherein the peptide or derivative comprises or consists of an amino acid sequence selected from (PEG12)KRRLLGLVFQSLCG(dA) (SEQ ID NO: 37); or RRIIGIVFQSLCG(dA) (SEQ ID NO: 43).

43. The method or the peptide, derivative, or salt for use of embodiment 39 or 40, wherein the peptide or derivative comprises or consists of an amino acid sequence selected from (K(PEG12))RRLLGLVFQSLCG(dA) (SEQ ID NO: 73); (PEG12)RRLLGLVFQSLCG(dA) (SEQ ID NO: 74); (PEG12)KRRIIGIVFQSLCG(dA) (SEQ ID NO: 75); (K(PEG12))RRIIGIVFQSLCG(dA) (SEQ ID NO: 76); and (PEG12}RRIIGIVFQSLCG(dA) (SEQ ID NO: 77).

44. The method or use of any one of embodiments 32-43, wherein the pro-inflammatory cytokine is selected from one or more of IL-1β, IL-2, IL-4, IL-5, IL-6, IL-9, IL-10, IL-12p70, IL-17α, IL17γ, IL-17A, IL-17C, IL-17E/IL-25, IL-17A/F, IL-23, IL-27p28/IL-30, IL-31, TNFα, IFNγ, IP-10, MCP-1, MIP-1α, MIP-2, MIP-3a and IL-8

45. The method or use of any one of embodiments 32-43, wherein the pro-inflammatory cytokine secretion is reduced.

46. A method of modulating activation of Ras, or phosphorylation of MEK1 or ERK1/2 comprising administering to the subject a peptide comprising either an amino acid sequence of Formula III′ or IV:

| | (III′) |
|---|---|
| (SEQ ID NO: 78) | |
| | X¹⁸-R-X¹⁹-X²⁰-X²¹ V-X²²-Q-X²³ L-X²⁴-G-X²⁵ |

wherein X¹⁸ is absent or if present is M or K; X¹⁹ is R or Aib; X²⁰ is absent or if present is -M-M-G- or Nle-Nle-G-; X²¹ is M or Nle; X²² is F, A or Y; X²³ is S, E or C; X²⁴ is E or C; X²⁵ is L, dA or -dA-K;

| | (IV) |
|---|---|
| (SEQ ID NO: 70) | |
| | X²⁶-RR-X²⁷-X²⁸ G-X²⁹-VFQ-X³⁰-LCG-(dA) |

wherein X²⁶ is absent or if present is K; X²⁷ is L or I; X²⁸ is L or I; X²⁹ is L or I; and X³⁰ is S or C;

or administering a derivative thereof comprising a C-terminal acid or amide, or a N-acetyl derivative thereof; or a pegylated derivative thereof; or administering a pharmaceutically acceptable salt thereof.

47. A peptide, or derivative thereof, or pharmaceutically acceptable salt thereof for use in modulating activation of Ras, or phosphorylation of MEK1 or ERK1/2, peptide comprising either an amino acid sequence of Formula III′ or IV:

| | (III′) |
|---|---|
| (SEQ ID NO: 78) | |
| | X¹⁸-R-X¹⁹-X²⁰-X²¹ V-X²²-Q-X²³ L-X²⁴-G-X²⁵ |

wherein X¹⁸ is absent or if present is M or K; X¹⁹ is R or Aib; X²⁰ is absent or if present is -M-M-G- or Nle-Nle-G-; X²¹ is M or Nle; X²² is F, A or Y; X²³ is S, E or C; X²⁴ is E or C; X²⁵ is L, dA or -dA-K;

| | (IV) |
|---|---|
| (SEQ ID NO: 70) | |
| | X²⁶-RR-X²⁷-X²⁸ G-X²⁹-VFQ-X³⁰-LCG-(dA) |

wherein X²⁶ is absent or if present is K; X²⁷ is L or I; X²⁸ is L or I; X²⁹ is L or I; and X³⁰ is S or C;

the derivative thereof comprising a C-terminal acid or amide, or a N-acetyl derivative thereof; or a pegylated derivative thereof.

48. A method of treating a subject having or suspected of having a disease or disorder selected from extravascular lung fluid accumulation, infectious disease or acute lung injury, the method comprising administering to the subject a peptide comprising an amino acid sequence of Formula I:

| | (I) |
|---|---|
| (SEQ ID NO: 1) | |
| | X¹- RX²-X³-X⁴-X⁵-X⁶-Q-X⁷-L-X⁸-X⁹ |

wherein X¹ is absent or if present is an amino acid having a polar side chain or a non-polar side chain; X² is an amino acid having a polar side chain or a non-polar side chain; X³ is absent or if present is one to three amino acids, each amino acid independently having a polar side chain or a non-polar side chain; X⁴ is an amino acid having a polar side chain or a non-polar side chain; X⁵ is an amino acid having a non-polar side chain; X⁶ is an amino acid having a polar side chain or a non-polar side chain; X⁷ is an amino acid having a polar side chain; X⁸ is an amino acid having a polar side chain; and X⁹ is absent or if present is one to three amino acids, each amino acid independently having a polar side chain or a non-polar side chain;

or administering an analog of said peptide having a deletion, insertion or substitution of one, two, three, or four amino acids; or administering a derivative that comprises a C-terminal acid or amide, or N-acetyl derivative thereof; or a pegylated derivative therefor; or administering a pharmaceutically acceptable salt thereof.

49. A peptide, or analog thereof, or derivative thereof, or pharmaceutically acceptable salt thereof for use in the treatment of a subject having or suspected of having a disease or disorder selected from extravascular lung fluid accumulation. infectious disease or acute lung injury, the peptide comprising an amino acid sequence of Formula

| | (I) |
|---|---|
| (SEQ ID NO: 1) | |
| | X¹- RX²-X³-X⁴-X⁵-X⁶-Q-X⁷-L-X⁸-X⁹ |

wherein X¹ is absent or if present is an amino acid having a polar side chain or a non-polar side chain; X² is an amino acid having a polar side chain or a non-polar side chain; X³ is absent or if present is one to three amino acids, each amino acid independently having a polar side chain or a non-polar side chain; X⁴ is an amino acid having a polar side chain or a non-polar side chain; X⁵ is an amino acid having a non-polar side chain; X⁶ is an amino acid having a polar side chain or a non-polar side chain; X⁷ is an amino acid having a polar side chain; X⁸ is an amino acid having a polar side chain; and X⁹ is absent or if present is one to three amino acids, each amino acid independently having a polar side chain or a non-polar side chain; or an analog of said peptide having a deletion, insertion or substitution of one, two, three, or four amino acids; wherein the derivative comprises a C-terminal acid or amide, or N-acetyl derivative thereof; or a pegylated derivative thereof.

50. The method or the peptide, analog, derivative, or salt for use of embodiment 48 or 49, wherein X¹ is M, K, or absent; X² is R or Aib; X³ is absent or is M, E, -MMG-, -LLG-, -II(dA)-, -Nle-Nle-G- or -IIG-; X⁴ is M, E, L, I or Nle; X⁵ is V, A or G; X⁶ is F, Y, A or E; X⁷ is C, S or E; X⁸ is C, S or E; and X⁹ is -GL, -G(dA), -G(dA)K, -(dA)L, G or absent.

51. The method or the peptide, analog, derivative, or salt for use of embodiment 48 or 49, wherein, in the peptide, analog, or derivative, X¹ is (PEG12)-K, and/or wherein X⁹ is -G(dA)-K(PEG12).

52. The method or the peptide, analog, derivative, or salt for use of embodiment 48 or 49, wherein X³ is absent or is -LLG-; X⁴ is L; X⁵ is V; or X⁸ is C or E.

53 The method or the peptide, analog, derivative, or salt for use of embodiment 48 or 49, wherein the peptide or derivative comprises or consists of an amino acid sequence selected from SEQ ID NOs: 2-63.

54. The method or the peptide, analog, derivative, or salt for use of embodiment 48 or 49, wherein the peptide or derivative comprises or consists of MRRMMGMVFQCLCGL (SEQ ID NO: 7); RRMMGMVFQCLCG(dA) (SEQ ID NO: 8); RRMMGMVYQCLCG(dA) (SEQ ID NO: 10); RRMMGMVAQCLCG(dA) (SEQ ID NO: 11); RRMMGMVFQELCG(dA) (SEQ ID NO: 13); RRMMGMVFQCLEG(dA) (SEQ ID NO: 14); RRMMGMVFQSLCG(dA) (SEQ ID NO: 15); RR(Nle)(Nle)G(Nle)VFQCLCG(dA) (SEQ ID NO: 18); (PEG12)KRRMMGMVFQCLCG(dA) (SEQ ID NO: 20); RRMMGMVFQCLCG(dA)K(PEG12) (SEQ ID NO: 21); RRMVYQCLCG(dA) (SEQ ID NO: 22); RRMMGMVAQCLEG(dA) (SEQ ID NO: 30); R(Aib)MMGMVFQSLCG(dA) (SEQ ID NO: 34); (PEG12)KRRMMGMVFQSLCG(dA) (SEQ ID NO: 36); (PEG12)KRRLLGLVFQSLCG(dA) (SEQ ID NO: 37); (PEG12)KRRIIGIVFQCLCG(dA) (SEQ ID NO: 42); or RRIIGIVFQSLCG(dA) (SEQ ID NO: 43).

55. The method or use of any one of embodiments 1-54, wherein the pharmaceutically acceptable salt is an acetate or hydrochoride salt.

56. The method or the peptide, analog, derivative, or salt for use of any one of embodiments 10, 19, 26, 37 or 42, wherein the peptide or derivative or salt comprises or consists of (PEG12)KRRLLGLVFQSLCG(dA) (SEQ ID NO: 37) acetate, RRMMGMVFQSLCG(dA) (SEQ ID NO: 15) acetate, (PEG12)KRRLLGLVFQSLCG(dA) (SEQ ID NO: 37) hydrochloride, or RRMMGMVFQSLCG(dA) (SEQ ID NO: 15) hydrochloride.

The peptides and their uses having been described, the following examples are offered by way of illustration, and not limitation.

### EXAMPLES

### Example 1

### Synthesis

The peptides are prepared via solid phase synthesis on a suitable resin using t-Boc or Fmoc chemistry or other well established techniques, (see for example: Stewart and Young, Solid Phase Peptide Synthesis, Pierce Chemical Co., Rockford, Ill., 1984; E. Atherton and R. C. Sheppard, Solid Phase Peptide Synthesis. A Practical Approach, Oxford-IRL Press, New York, 1989; Greene and Wuts, “Protective Groups in Organic Synthesis”, John Wiley & Sons, 1999, Florencio Zaragoza Dorwald, “Organic Synthesis on solid Phase”, Wiley-VCH Verlag GmbH, 2000, and “Fmoc Solid Phase Peptide Synthesis”, Edited by W. C. Chan and P. D. White, Oxford University Press, 2000) by a method similar to that described below, unless specified otherwise.

Solid phase synthesis is initiated by attaching an N-terminally protected amino acid with its carboxy terminus to an inert solid support carrying a cleavable linker. This solid support can be any polymer that allows coupling of the initial amino acid, e.g. a Pam resin, trityl resin, a chlorotrityl resin, a Wang resin or a Rink resin in which the linkage of the carboxy group (or carboxamide for Rink resin) to the resin is sensitive to acid (when Fmoc strategy is used). The polymer support is stable under the conditions used to deprotect the α-amino group during the peptide synthesis. After the first amino acid has been coupled to the solid support, the α-amino protecting group of this amino acid is removed. The remaining protected amino acids are then coupled one after the other in the order represented by the peptide sequence using appropriate amide coupling reagents, for example BOP (benzotriazol-1-yl-oxy-tris-(dimethylamino)-phosphonium), HBTU (2-(1H-benzotriazol-1-yl)-1,1,3,3-tetramethyl-uronium), HATU (O-(7-azabenzotriazol-1-yl-oxy-tris-(dimethylamino)-phosphonium) or DIC (N,N′-diisopropylcarbodiimide)/HOBt (1-hydroxybenzotriazol), wherein BOP, HBTU and HATU are used with tertiary amine bases. Alternatively, the liberated N-terminus can be functionalized with groups other than amino acids, for example carboxylic acids, etc. Usually, reactive side-chain groups of the amino acids are protected with suitable blocking groups. These protecting groups are removed after the desired peptides have been assembled. They are removed concomitantly with the cleavage of the desired product from the resin under the same conditions. Protecting groups and the procedures to introduce protecting groups can be found in Protective Groups in Organic Synthesis, 3d ed., Greene, T. W. and Wuts, P. G. M., Wiley & Sons (New York: 1999). In some cases, it might be desirable to have side-chain protecting groups that can selectively be removed while other side-chain protecting groups remain intact. In this case the liberated functionality can be selectively functionalized. For example, a lysine may be protected with an ivDde protecting group (S. R. Chhabra et al., Tetrahedron Lett. 39, (1998), 1603) which is labile to a very nucleophilic base, for example 4% hydrazine in DMF (dimethyl formamide). Thus, if the N-terminal amino group and all side-chain functionalities are protected with acid labile protecting groups, the ivDde ([1-(4,4-dimethyl-2,6-dioxocyclohex-1-ylidene)-3-methylbutyl) group can be selectively removed using 4% hydrazine in DMF and the corresponding free amino group can then be further modified, e.g. by acylation. The lysine can alternatively be coupled to a protected amino acid and the amino group of this amino acid can then be deprotected resulting in another free amino group which can be acylated or attached to further amino acids. Finally, the peptide is cleaved from the resin. This can be achieved by using HF or King's cocktail (D. S. King, C. G. Fields, G. B. Fields, Int. J. Peptide Protein Res. 36, 1990, 255-266). The raw material can then be purified by chromatography, e.g. preparative RP-HPLC, if necessary.

Those peptides, analogs or derivatives which include non-natural amino acids and/or a covalently attached N-terminal mono- or dipeptide mimetic may be produced as described in the experimental part. Or see e.g., Hodgson et al: “The synthesis of peptides and proteins containing non-natural amino acids”, and Chemical Society Reviews, vol. 33, no. 7 (2004), p. 422-430.

The peptides are prepared according to the below-mentioned peptide synthesis and the sequences as presented in the Table 1 can be prepared similar to the below-mentioned synthesis, unless specified otherwise.

One method of peptide synthesis is by Fmoc chemistry on a microwave-based Liberty peptide synthesizer (CEM Corp., North Carolina). The resin is Tentagel S RAM with a loading of about 0.25 mmol/g or PAL-ChemMatrix with a loading of about 0.43 mmol/g or PAL AM matrix with a loading of 0.5-0.75 mmol/g. The coupling chemistry is DIC/HOAt or DIC/Oxyma in NMP or DMF using amino acid solutions of 0.3 M and a molar excess of 6-8 fold. Coupling conditions are 5 minutes at up to 70° C. Deprotection is with 10% piperidine in NMP at up to 70° C. The protected amino acids used are standard Fmoc-amino acids (supplied from e.g. Anaspec or Novabiochem or Protein Technologies).

Another method of peptide synthesis is by Fmoc chemistry on a Prelude peptide synthesizer (Protein Technologies, Arizona). The resin is Tentagel S RAM with a loading of about 0.25 mmol/g or PAL-ChemMatrix with a loading of about 0.43 mmol/g or PAL AM with a loading of 0.5-0.75 mmol/g. The coupling chemistry is DIC/HOAt or DIC/Oxyma in NMP or DMF using amino acid solutions of 0.3 M and a molar excess of 6-8 fold. Coupling conditions are single or double couplings for 1 or 2 hours at room temperature. Deprotection is with 20% piperidine in NMP. The protected amino acids used are standard Fmoc-amino acids (supplied from e.g. Anaspec or Novabiochem or Protein Technologies). The crude peptides are purified such as by semipreparative HPLC on a 20 mm×250 mm column packed with either 5 um or 7 um C-18 silica. Peptide solutions are pumped onto the HPLC column and precipitated peptides are dissolved in 5 ml 50% acetic acid H₂O and diluted to 20 ml with H₂O and injected on the column which then is eluted with a gradient of 40-60% CH₃CN in 0.1% TFA 10 ml/min during 50 min at 40° C. The peptide containing fractions are collected. The purified peptide is lyophilized after dilution of the eluate with water.

All peptides with C terminal amides described herein are prepared by a method similar to that described below unless specified otherwise. MBHA resin (4-methylbenzhydrylamine polystyrene resin is used during peptide synthesis. MBHA resin, 100-180 mesh, 1% DVB cross-linked polystyrene; loading of 0.7-1.0 mmol/g), Boc-protected and Fmoc protected amino acids can be purchased from Midwest Biotech. The solid phase peptide syntheses using Boc-protected amino acids are performed on an Applied Biosystem 430A Peptide Synthesizer. Fmoc protected amino acid synthesis is performed using the Applied Biosystems Model 433 Peptide Synthesizer.

Synthesis of the peptides is performed on the Applied Biosystem Model 430A Peptide Synthesizer. Synthetic peptides are constructed by sequential addition of amino acids to a cartridge containing 2 mmol of Boc protected amino acid. Specifically, the synthesis is carried out using Boc DEPBT-activated single couplings. At the end of the coupling step, the peptidyl-resin is treated with TFA to remove the N-terminal Boc protecting group. It is washed repeatedly with DMF and this repetitive cycle is repeated for the desired number of coupling steps. After the assembly, the sidechain protection, Fmoc, is removed by 20% piperidine treatment and acylation was conducted using DIC. The peptidyl-resin at the end of the entire synthesis is dried by using DCM, and the peptide is cleaved from the resin with anhydrous HF. The peptidyl-resin is treated with anhydrous HF, and this typically yielded approximately 350 mg (˜50% yield) of a crude deprotected-peptide. Specifically, the peptidyl-resin (30 mg to 200 mg) is placed in the hydrogen fluoride (HF) reaction vessel for cleavage. 500 μL of p-cresol was added to the vessel as a carbonium ion scavenger. The vessel is attached to the HF system and submerged in the methanol/dry ice mixture. The vessel is evacuated with a vacuum pump and 10 ml of HF is distilled to the reaction vessel. This reaction mixture of the peptidyl-resin and the HF is stirred for one hour at 0° C., after which a vacuum is established and the HF is quickly evacuated (10-15 min). The vessel is removed carefully and filled with approximately 35 ml of ether to precipitate the peptide and to extract the p-cresol and small molecule organic protecting groups resulting from HF treatment. This mixture is filtered utilizing a Teflon filter and repeated twice to remove all excess cresol. This filtrate is discarded. The precipitated peptide dissolves in approximately 20 ml of 10% acetic acid (aq). This filtrate, which contained the desired peptide, is collected and lyophilized.

### Example 2

### β-Arrestin Recruitment in Cultured Apelin Receptor Overexpressing CHO-K1 Cells

The effect of the peptides on activation of Apelin Receptor (APJ) can be assessed using an assay to monitor β-Arrestin recruitment in cultured cells overexpressing APJ such as CHO-K1, derived from Chinese hamster ovary. β-Arrestin recruitment assays were performed by Eurofins-DiscoverX (Fremont, Calif.) using CHO-K1 AGTRL1 β-Arrestin cell line (co-expressing ProLink tagged human APJ and Enzyme Acceptor tagged β-Arrestin) and PathHunter detection kit. Peptides were initially prepared either as 10 mM stock in DMSO and used at a final concentration of 10 μM (0.1% DMSO). CHO-K1 AGTRL1 β-Arrestin cells were seed onto 384-well plates in standard medium. After overnight culture, the medium was replaced with buffer containing 500 nM Apelin-13 (positive control) or 10 μM peptide. Following 90 min. incubation at 37° C., β-Arrestin recruitment in response to various treatments was quantified using a chemiluminescent complementation reporter assay to measure association of tagged human APJ (ProLink tag) and tagged β-Arrestin (Enzyme Acceptor tag). Data are presented as percent of Apelin-13 response (100%) with each data point representing the average of duplicates. The results are shown in Table 4. This example illustrates the activity of various peptides as APJ agonists.

TABLE 4
β -Arrestin Recruitment in Cultured CHO-K1 AGTRL1 β-Arrestin Cells

| SEQ ID NO: | Percent of Apelin-13 Control Activity |
|---|---|
| 7 | 91 |
| 8 | 17 |
| 10 | 3 |
| 11 | 0 |
| 12 | −1 |
| 13 | 1 |
| 14 | 0 |
| 15 | 40 |
| 16 | 0 |
| 17 | 10 |
| 18 | 1 |
| 19 | 1 |
| 20 | 1 |
| 21 | 1 |
| 22 | 0 |
| 23 | 0 |
| 24 | −1 |
| 25 | −1 |
| 26 | 0 |
| 27 | −1 |
| 28 | −1 |
| 29 | 0 |
| 30 | −1 |
| 31 | 0 |
| 32 | 0 |
| 33 | 9 |
| 34 | 8 |
| 35 | 17 |
| 36 | 63 |
| 37 | 12 |
| 38 | 0 |
| 39 | 0 |
| 40 | 0 |
| 41 | 0 |
| 42 | 8 |
| 43 | 82 |
| 44 | 0 |
| 45 | 0 |
| 46 | 0 |
| 47 | 1 |
| 48 | 1 |
| 49 | 1 |
| 50 | 0 |
| 51 | 0 |
| 52 | 0 |
| 53 | 1 |
| 54 | 1 |
| 55 | 1 |
| 56 | 0 |
| 57 | 0 |
| 58 | 0 |
| 59 | 0 |
| 60 | 0 |
| 61 | 5 |
| 62 | 2 |
| 63 | 9 |

### Example 3

### cAMP Levels in Cultured Apelin Receptor Overexpressing CHO-K1 Cells

The effect of the peptides on activation of Apelin Receptor (APJ) can be assessed using an assay to monitor inhibition of cAMP expression in cultured cells overexpressing APJ such as CHO-K1, derived from Chinese hamster ovary. Peptides were initially prepared as 30 mM stock in DMSO and diluted to 3 mM in H₂O or directly as 3 mM stock in H₂O; used at a final concentration of 10 μM (0-0.1% DMSO). Forskolin was used as a highly potent inducer of cAMP expression. CHO-K1 AGTRL1 Gi cells stably overexpressing APJ were purchased from Eurofins-DiscoverX (Fremont, Calif.). CHO-K1 AGTRL1 Gi cells were seeded onto 384-well plates in standard culture medium (F12K+10% Fetal Bovine Serum+antibiotics) at 10,000 cells/well and allowed to adhere overnight at 37° C. in a humidified atmosphere of 5% CO₂/95% air. After overnight culture, the medium was replaced with buffer containing 10 μM forskolin (to increase cAMP expression) and either 500 nM Pyr-Apelin-13 (inhibits cAMP accumulation) or 10 μM peptide. Following 30 min incubation at 37° C., cAMP levels in response to various treatments were quantified using HitHunter cAMP kit according to manufactures protocol (Eurofins-DiscoverX); chemiluminescent signal was measured using a Cytation 3 plate reader (BioTek, Winooski, Vt.). Data are presented as percent of Pyr-Apelin-13 response (100%) with each data point representing the average of triplicates. The results are shown in Table 5. This example illustrates the activity of various peptides as APJ agonists.

TABLE 5
cAMP Levels in Cultured CHO-K1 AGTRL1 Gi Cells

| SEQ ID NO: | Percent of Pyr-Apelin-13 Control Activity |
|---|---|
| 7 | 109 |
| 8 | 64 |
| 10 | 70 |
| 11 | 49 |
| 12 | 24 |
| 13 | 49 |
| 14 | 39 |
| 15 | 105 |
| 17 | 26 |
| 18 | 30 |
| 19 | 27 |
| 20 | 37 |
| 21 | 34 |
| 22 | 62 |
| 23 | −4 |
| 24 | 16 |
| 25 | 3 |
| 26 | 29 |
| 27 | 9 |
| 28 | 3 |
| 29 | 21 |
| 30 | 31 |
| 31 | 2 |
| 32 | 4 |
| 33 | 27 |
| 34 | 61 |
| 35 | 8 |
| 36 | 101 |
| 37 | 53 |
| 38 | 12 |
| 39 | −13 |
| 40 | 19 |
| 41 | 15 |
| 42 | 33 |
| 43 | 44 |
| 44 | 5 |
| 45 | 10 |
| 46 | 11 |
| 47 | −6 |
| 48 | 15 |
| 49 | 14 |
| 50 | 8 |
| 51 | 24 |
| 52 | 15 |
| 53 | 6 |
| 54 | 20 |
| 55 | −7 |
| 56 | −2 |
| 57 | −1 |
| 58 | 5 |
| 59 | −2 |
| 60 | 16 |
| 61 | 1 |
| 62 | 27 |
| 63 | 21 |

### Example 4

### Metabolic Stability in Plasma

The metabolic stability of the peptides can be assessed in vitro by incubation in plasma and determination of the amount of peptide remaining over time. Peptides (50 μM) were incubated in pooled plasma from mice, monkeys, and humans at 37° C. Samples were removed at intervals up to 3 hours and immediately analyzed for the concentration of intact peptide by LC/MS/MS. The percent of peptide remaining in plasma at each time point was calculated relative to the initial peak area. The percent of initial peptide remaining at 30 minutes after incubation in pooled human plasma is shown in Table 6.

TABLE 6
Stability of Peptides in Human Plasma

| | Percent Remaining in Human |
|---|---|
| SEQ ID NO: | Plasma at 30 min |
| 7 | 52.4 |
| 8 | 49.4 |
| 9 | 92.5 |
| 10 | 65.0 |
| 11 | 97.9 |
| 12 | 95.2 |
| 13 | 41.0 |
| 14 | 78.1 |
| 15 | 24.3 |
| 16 | 26.4 |
| 17 | 97.4 |
| 18 | 64.2 |
| 19 | 73.9 |
| 20 | 75.3 |
| 21 | 29.5 |
| 22 | 98.5 |
| 23 | 43.6 |
| 24 | 48.4 |
| 25 | 100 |
| 26 | 18.3 |
| 27 | 100 |
| 28 | 45.8 |
| 29 | 6.2 |
| 30 | 14.9 |
| 31 | 100 |
| 32 | 5.6 |
| 33 | 24.0 |
| 35 | 73.2 |
| 38 | 60.1 |
| 39 | 98.3 |
| 40 | 31.9 |
| 41 | 100 |
| 45 | 90.8 |
| 46 | 100 |
| 47 | 86.1 |
| 48 | 92.0 |
| 49 | 8.1 |
| 50 | 87.7 |
| 51 | 100 |
| 52 | 97.5 |
| 53 | 85.8 |
| 54 | 8.5 |
| 55 | 100 |
| 56 | 92.1 |
| 57 | 91.9 |
| 58 | 69.5 |
| 59 | 62.9 |
| 60 | 95.0 |
| 62 | 30.3 |

### Example 5

### cAMP Levels in Cultured Apelin Receptor Overexpressing CHO-K1 Cells

The effect of the peptides on activation of Apelin Receptor (APJ) can be assessed using an assay to measure inhibition of forskolin-stimulated cAMP accumulation in cultured cells overexpressing APJ such as CHO-K1 cells. CHO-K1 AGTRL1 Gi cells stably overexpressing APJ were purchased from Eurofins-DiscoverX (Fremont, Calif.), seeded onto 384-well plates in standard culture medium at 10,000 cells/well, and allowed to adhere overnight at 37° C. in a humidified atmosphere of 5% CO₂/95% air. After overnight culture, the medium was replaced with buffer containing 10 μM forskolin to increase cAMP expression together with either Pyr-Apelin-13 (0.025-167 nM) or peptides of the invention (0.005-30 μM). Following 30 min incubation at 37° C., cAMP levels in response to various treatments were quantified using HitHunter cAMP kit according to manufactures protocol (Eurofins-DiscoverX); chemiluminescent signal was measured using a Cytation 3 plate reader (BioTek, Winooski, Vt.). Data were plotted as mean (SD) percent of Pyr-Apelin-13 response (100%) based on the average of 2-3 values. IC₅₀ values were determined by GraphPad Prism software (Graph Pad Software, San Diego, Calif.). Data are mean (SD) n=2-3 for all data points. The IC₅₀ values are shown in Table 7.

TABLE 7

| SEQ ID NO: | IC50 (M) |
|---|---|
| Apelin-13 | 1.763 × 10⁻⁹ |
| 15 | 4.492 × 10⁻⁶ |
| 36 | 1.602 × 10⁻⁶ |
| 37 | 2.499 × 10⁻⁶ |
| 42 | 4.382 × 10⁻⁶ |
| 43 | 2.069 × 10⁻⁶ |
| 7 | 1.243 × 10⁻⁶ |

### Example 6

### LPS-Induced Acute Lung Injury Model in Mouse

The effect of the peptides of the current invention on acute lung injury can be assessed in an LPS-induced acute lung injury mouse model by monitoring outcomes such as lung weight, fluid accumulation, cytokine secretion in blood or bronchoalveolar lavage fluid (BALF), neutrophil infiltration into lung tissues, and assessment of acute lung injury score by histopathology. Acute lung injury was induced in male C57BL/6 mice (6 to 8 weeks old) by intratracheal administration of 40 μL lipopolysaccharide (LPS) (Sigma Aldrich, St. Louis, Mo.) in PBS at 5 mg/kg. Control animals received intratracheal PBS alone. Animals (n=8 per treatment group) induced with LPS received intraperitoneal treatment with: PBS (vehicle control); apelin-13 (Cayman Chemical, Ann Arbor, Mich.) at 10 nmol/kg in water (positive control); or test peptides at 5 or 15 mg/kg in water. Treatments were administered 1 h prior to LPS for animals designated for sacrifice at 4 h after LPS, or at both 1 h prior to and 6 h after LPS for animals designated for sacrifice at 24 h after LPS. At 4 or 24 hours post LPS administration, tissues were harvested. Lungs were weighed, flushed with Hanks Buffer to provide bronchoalveolar lavage fluid (BALF). Lungs were fixed in 10% neutral buffered formalin (NBF) for histopathology. Tissue slides were stained with hematoxylin and eosin (H&E) and evaluated and scored with light microscopy of 5 representative microscopic fields (100× magnification) scoring on a 0-5 scale using standard methods (Matute-Bello G et al 2011. An Official American Thoracic Society Workshop Report: Features and Measurements of Experimental Acute Lung Injury in Animals. Am J Respir Cell Mol Biol 44:725-38). The average score for five fields was calculated for each animal. Levels of pro-inflammatory cytokines in BALF were determined using a meso scale discovery (MSD) system. Data for test peptides were compared to the vehicle control group. Lung weights were normalized to body weight. Table 8 compares the normalized lung weights following LPS induction and treatment with PBS (vehicle control), apelin-13 (positive control) or peptides. Table 9 compares the levels of pro-inflammatory cytokines in BALF following LPS induction and treatment with PBS (vehicle control), apelin-13 (positive control) or peptides. Table 10 compares the histopathology scores for infiltration of neutrophils into alveoli and interstitial lung tissue and the composite lung injury score following LPS induction and treatment with PBS (vehicle control), apelin-13 (positive control) or peptides. Treatment of LPS-induced animals with the peptides of the invention resulted in a decrease in lung weight (decreased fluid accumulation), a decrease in levels of pro-inflammatory cytokines in BALF, a decrease in neutrophil infiltration in alveoli and interstitial lung tissue, and a decrease in composite lung injury score, relative to induction with LPS and treatment with vehicle. The lung weights are provided in Table 8. The pro-inflammatory Cytokine secretion Levels in Bronchoalveolar Lavage Fluid are provided in Table 9. The corresponding histopathology scores are provided in Table 10.

TABLE 8
Normalized Lung Weight at 4 or 24 h After LPS-Induced
Acute Lung Injury

| | Peptide | Mean (SEM) Lung Weight (g) | |
|---|---|---|---|
| Treatment | Dose | 4 h | 24 h |
| Control (no LPS) | N/A | 0.95 (0.01) | 1.00 (0.03) |
| LPS/Vehicle | N/A | 1.11 (0.02) | 1.44 (0.04) |
| LPS/Apelin-13 | 10 nmol/kg | 1.03 (0.03)** | 1.32 (0.03)* |
| LPS/SEQ ID NO: 15 | 5 mg/kg | 1.03 (0.02)* | 1.29 (0.04)** |
| LPS/SEQ ID NO: 15 | 15 mg/kg | 0.98 (0.02)** | 1.33 (0.03)* |

Statistical significance versus LPS/Vehicle by Student's t-test: *p < 0.05, **p < 0.01.

TABLE 9
Pro-inflammatory Cytokine Levels in Bronchoalveolar Lavage Fluid of Mice
4 Hours After LPS-Induced Acute Lung Injury

| | | Mean (SEM) Pro-inflammatory Cytokine Level (pg/mL) | | | | | | | |
|---|---|---|---|---|---|---|---|---|---|
| | Peptide | | | | KC/ | | IL- | IL- | |
| Treatment | Dose | TNFα | IL-1β | IFNγ | GRO | IL-6 | 17-A | 17-C | IL-23 |
| Control | N/A | 8.67 | 0.38 | 0.00 | 19.6 | 12.8 | 0.00 | 0.07 | 0.11 |
| (no LPS) | | (1.21) | (0.20) | (0.00) | (6.04) | (2.89) | (0.00) | (0.05) | (0.04) |
| LPS/Vehicle | N/A | 15300 | 101 | 0.04 | 2570 | 3196 | 0.40 | 0.87 | 0.92 |
| | | (5950) | (14.1) | (0.03) | (421) | (576) | (0.09) | (0.12) | (0.11) |
| LPS/Apelin-13 | 10 | 15500 | 123 | 0.04 | 3210 | 4540 | 0.87 | 1.84 | 0.68 |
| | nmol/kg | (5080) | (16.9) | (0.02) | (328) | (954) | (0.39) | (0.32) | (0.15) |
| LPS/SEQ ID | 5 mg/kg | 18200 | 78.9 | 0.00 | 1980 | 2840 | 0.22 | 0.86 | 0.75 |
| NO: 15 | | (7820) | (14.3) | (0.00) | (412) | (684) | (0.09) | (0.20) | (0.18) |
| LPS/SEQ ID | 15 mg/kg | 5680 | 77.2 | 0.00 | 1810 | 2017 | 0.08 | 0.72 | 0.33 |
| NO: 15 | | (1100) | (1.0) | (0.00) | (252) | (229) | (0.03) | (0.16) | (0.09) |

TABLE 10
Histopathology Scores in Mice 24 Hours After LPS-Induced
Acute Lung Injury

| | | Mean (SEM) | Mean (SEM) | Mean |
|---|---|---|---|---|
| | | Alveolar | Interstitial | (SEM) |
| | | Neutrophil | Neutrophil | Composite |
| | Peptide | Infiltration | Infiltration | Lung Injury |
| Treatment | Dose | Score | Score | Score |
| Control (no LPS) | N/A | 0.00 (0.00) | 0.00 (0.00) | 0.00 (0.00) |
| LPS/Vehicle | N/A | 2.05 (0.15) | 1.70 (0.14) | 3.95 (0.23) |
| LPS/Apelin-13 | 10 nmol/kg | 1.40 (0.21) | 1.13 (0.11) | 2.58 (0.29) |
| LPS/SEQ ID | 5 mg/kg | 1.73 (0.08) | 1.50 (0.13) | 3.38 (0.19) |
| NO: 15 | | | | |
| LPS/SEQ ID | 5 mg/kg | 1.98 (0.18) | 1.60 (0.14) | 3.65 (0.28) |
| NO: 36 | | | | |
| LPS/SEQ ID | 5 mg/kg | 1.50 (0.14) | 1.30 (0.11) | 2.83 (0.23) |
| NO: 37 | | | | |

### Example 7

### LPS-Induced Acute Lung Injury Model in Mouse

The effect of the peptides of the current invention on acute lung injury can be assessed in an LPS-induced acute lung injury mouse model by monitoring outcomes such as lung weight, fluid accumulation, cytokine secretion in blood or bronchoalveolar lavage fluid (BALF). Acute lung injury was induced in male C57BL/6 mice (6 to 8 weeks old) by intratracheal administration of 40 μL lipopolysaccharide (LPS) (Sigma Aldrich, St. Louis, Mo.) in PBS at 5 mg/kg. Control animals received intratracheal PBS alone. Animals (n=8 per treatment group) induced with LPS received intraperitoneal treatment with: PBS (vehicle control) or test peptides at 5 mg/kg in water. Treatments were administered 1 h prior to LPS. At 4 h post LPS administration, tissues were harvested. Lungs were weighed, flushed with Hanks Buffer to provide bronchoalveolar lavage fluid (BALF). Data for test peptides were compared to the vehicle control group. Table 11 compares the lung weights following LPS induction and treatment with PBS (vehicle control) or peptides. Table 12 compares the levels of pro-inflammatory cytokines in BALF following LPS induction and treatment with PBS (vehicle control) or peptides. Treatment of LPS-induced animals with the peptides of the invention resulted in a decrease in lung weight (decreased fluid accumulation) and a decrease in levels of pro-inflammatory cytokines in BALF, relative to induction with LPS and treatment with vehicle.

TABLE 11
Lung Weight at 4 h After LPS-Induced Acute Lung Injury

| | Peptide | Mean (SEM) |
|---|---|---|
| Treatment | Dose | Lung Weight (g) |
| Control (no LPS) | N/A | 0.228 (0.003) |
| LPS/Vehicle | N/A | 0.298 (0.007) |
| LPS/SEQ ID NO: | 5 mg/kg | 0.272 (0.005)* |
| 15 | | |
| LPS/SEQ ID NO: | 5 mg/kg | 0.259 (0.004)*** |
| 37 | | |

Statistical significance versus LPS/Vehicle by Student's t-test: *p < 0.05, ***p < 0.001.

TABLE 12
Pro-inflammatory Cytokine Levels in Bronchoalveolar Lavage Fluid of Mice
4 Hours After LPS-Induced Acute Lung Injury

| | | Mean (SEM) Pro-inflammatory Cytokine Level (pg/mL) | | | | | | | |
|---|---|---|---|---|---|---|---|---|---|
| | Peptide | | | | KC/ | | | | |
| Treatment | Dose | TNFα | IL-1β | IFNγ | GRO | IL-6 | IL-2 | IL-4 | IL-9 |
| Control | N/A | 0.17 | 0.00 | 0.01 | 2.50 | 0.00 | 0.00 | 0.03 | 0.32 |
| (no LPS) | | (0.04) | (0.00) | (0.00) | (0.37) | (0.00) | (0.00) | (0.02) | (0-14) |
| LPS/Vehicle | N/A | 7110 | 129 | 0.36 | 3270 | 4710 | 2.52 | 2.07 | 7.74 |
| | | (896) | (20.9) | (0.07) | (144) | (402) | (0.64) | (0.33) | (0-41) |
| LPS/SEQ ID | 5 mg/kg | 4950 | 87.2 | 0.34 | 2890 | 5020 | 1.86 | 1.85 | 5.84 |
| NO: 15 | | (751) | (8.98) | (0.10) | (250) | (1750) | (0.85) | (0.43) | (0.96) |
| LPS/SEQ ID | 5 mg/kg | 3850 | 72.5 | 0.13 | 2660 | 3080 | 0.60 | 0.86 | 4.74 |
| NO: 37 | | (548) | (8.17) | (0.03) | (142) | (257) | (0.29) | (0.11) | (0.65) |

### Example 8

### LPS-Induced Acute Lung Injury Model in Mouse

The effect of the peptides of the current invention on acute lung injury can be assessed in an LPS-induced acute lung injury mouse model by monitoring outcomes such as lung weight, fluid accumulation, cytokine secretion in blood or bronchoalveolar lavage fluid (BALF), neutrophil infiltration into lung tissues, and assessment of acute lung injury score by histopathology. Acute lung injury was induced in male C57BL/6 mice (6 to 8 weeks old) by intratracheal administration of 40 μL lipopolysaccharide (LPS) (Sigma Aldrich, St. Louis, Mo.) in PBS at 5 mg/kg. Control animals received intratracheal PBS alone. Animals (n=8 per treatment group) induced with LPS received intraperitoneal treatment with: PBS (vehicle control) or test peptides at 5 or 15 mg/kg in water. Treatments were administered 1 h prior to LPS for animals designated for termination at 4 h after LPS, or at both 1 h prior to and 6 h after LPS for animals designated for termination at 24 h after LPS. At 4 or 24 hours post-LPS administration, tissues were harvested. Lungs were weighed and flushed with Hanks Buffer to provide bronchoalveolar lavage fluid (BALF). Lungs collected at 24 h were fixed in 10% neutral buffered formalin (NBF) for histopathology. Tissue slides were stained with hematoxylin and eosin (H&E) and evaluated and scored with light microscopy of 5 representative microscopic fields (100× magnification) scoring on a 0-5 scale using standard methods (Matute-Bello G et al 2011. An Official American Thoracic Society Workshop Report: Features and Measurements of Experimental Acute Lung Injury in Animals. Am J Respir Cell Mol Biol 44:725-38). The average score for five fields was calculated for each animal. Levels of pro-inflammatory cytokines in BALF at 4 h were determined using a meso scale discovery (MSD) system. Data for test peptides were compared to the vehicle control group. Table 13 compares the lung weights following LPS induction and treatment with PBS (vehicle control), or peptides. Table 14 compares the levels of pro-inflammatory cytokines in BALF following LPS induction and treatment with PBS (vehicle control) or peptides. Table 15 compares the histopathology scores for infiltration of neutrophils into alveoli and interstitial lung tissue and the composite lung injury score following LPS induction and treatment with PBS (vehicle control) or peptides. Treatment of LPS-induced animals with the peptides of the invention resulted in a decrease in lung weight (decreased fluid accumulation), a decrease in levels of pro-inflammatory cytokines in BALF, a decrease in neutrophil infiltration in alveoli and interstitial lung tissue, and a decrease in composite lung injury score, relative to induction with LPS and treatment with vehicle.

TABLE 13
Lung Weight at 4 h or 24 h After LPS-Induced Acute Lung Injury

| | Peptide | Mean (SEM) Lung Weight (g) | |
|---|---|---|---|
| Treatment¹ | Dose | 4 h | 24 h |
| Control (no LPS) | N/A | 0.221 (0.003) | 0.216 (0.005) |
| LPS/Vehicle | N/A | 0.287 (0.006) | 0.310 (0.004) |
| LPS/SEQ ID NO: 15 | 5 mg/kg | 0.269 (0.005)** | 0.295 (0.005)* |
| LPS/SEQ ID NO: 15 | 15 mg/kg | 0.271 (0.006)* | 0.287 (0.008)* |
| LPS/SEQ ID NO: 37 | 5 mg/kg | 0.279 (0.006) | 0.286 (0.008)* |
| LPS/SEQ ID NO: 37 | 15 mg/kg | 0.280 (0.005) | 0.284 (0.007)* |

¹Peptides were administered as HCl salts.
Statistical significance versus LPS/Vehicle by Student's t-test: *p < 0.05, **p < 0.01.

TABLE 14
Pro-inflammatory Cytokine Levels in Bronchoalveolar Lavage Fluid of Mice
4 Hours After LPS-Induced Acute Lung Injury

| | Mean (SEM) Pro-inflammatory Cytokine Level (pg/mL) | | | | | |
|---|---|---|---|---|---|---|
| | Control (No | LPS + | LPS + SEQ ID NO: 15 | | LPS + SEQ ID NO: 37 | |
| Cytokine | LPS) | Vehicle | 5 mg/kg¹ | 15 mg/kg¹ | 5 mg/kg¹ | 15 mg/kg¹ |
| IFNγ | 0.06 (0.02) | 0.82 (0.15) | 0.46 (0.06) | 0.44 (0.112) | 0.37 (0.07) | 0.42 (0.10) |
| IL-1β | 0.21 (0.15) | 112 (11.6) | 66.2 (6.64) | 57.1 (11.1) | 68.3 (12.6) | 91.5 (20.0) |
| IL-2 | 0.13 (0.08) | 4.92 (0.80) | 4.07 (0.59) | 2.86 (0.57) | 2.83 (0.45) | 3.36 (0.55) |
| IL-4 | 0.04 (0.02) | 3.01 (0.78) | 3.22 (0.77) | 2.19 (0.51) | 1.63 (0.37) | 1.75 (0.28) |
| IL-5 | 0.03 (0.02) | 28.0 (3.87) | 8.53 (1.86) | 10.8 (3.21) | 8.14 (1.35) | 13.2 (2.05) |
| IL-6 | 0.00 (0.00) | 16400 (2250) | 10400 (1550) | 9670 (2170) | 9380 (1380) | 11600 (1360) |
| IL-10 | 0.00 (0.00) | 51.0 (5.13) | 31.2 (5.61) | 39.6 (13.2) | 32.2 (5.31) | 31.5 (3.76) |
| IL-12p70 | 0.00 (0.00) | 175 (41.8) | 130 (27.6) | 109 (30.1) | 65.2 (15.9) | 80.0 (21.8) |
| KC/GRO | 3.22 (0.446) | 3410 (48.1) | 3070 (276) | 3140 (135) | 3120 (196) | 3260 (142) |
| TNFα | 0.95 (0.11) | 4020 (304) | 4600 (474) | 3270 (247) | 4070 (518) | 3910 (349) |
| IL-17A | 0.02 (0.01) | 5.62 (1.23) | 2.20 (0.275) | 3.11 (1.42) | 2.22 (0.741) | 2.82 (0.639) |
| IL-17C | 0.35 (0.10) | 1.04 (0.21) | 0.83 (0.11) | 1.25 (0.25) | 0.66 (0.04) | 0.69 (0.13) |
| IL-17E/IL-25 | 0.19 (0.15) | 0.35 (0.24) | 0.16 (0.10) | 0.00 (0.00) | 0.18 (0.10) | 0.44 (0.29) |
| IL-23 | 0.00 (0.00) | 0.45 (0.19) | 0.06 (0.04) | 0.06 (0.06) | 0.08 (0.07) | 0.06 (0.05) |
| IL-27p28/IL-30 | 0.00 (0.00) | 29.8 (3.78) | 18.4 (3.36) | 13.8 (2.84) | 13.7 (2.44) | 15.5 (2.42) |
| IL-31 | 0.41 (0.41) | 0.41 (0.41) | 0.66 (0.66) | 0.00 (0.00) | 0.00 (0.00) | 0.00 (0.00) |
| IL-9 | 0.91 (0.37) | 10.1 (0.83) | 12.1 (1.93) | 7.29 (1.31) | 7.84 (0.67) | 7.89 (1.31) |
| IL-17A/F | 0.41 (0.16) | 7.97 (0.91) | 5.11 (0.80) | 4.72 (0.76) | 7.45 (2.46) | 4.48 (0.59) |
| IP-10 | 1.95 (1.01) | 3430 (282) | 2930 (270) | 2190 (430) | 2290 (341) | 3020 (547) |
| MCP-1 | 0.35 (0.27) | 2370 (282) | 1430 (147) | 1110 (233) | 1310 (225) | 1590 (178) |
| MIP-1α | 0.34 (0.13) | 5700 (25.8) | 5680 (25.5) | 5540 (55.0) | 5660 (50.6) | 5490 (35.1) |
| MIP-2 | 1.92 (0.31) | 3230 (25.3) | 3190 (23.2) | 3100 (141) | 3230 (25.1) | 3160 (34.2) |
| MIP-3α | 8.80 (1.03) | 1480 (133) | 1220 (129) | 1040 (64.2) | 983 (75.1) | 825 (46.9) |

¹Peptides were administered as HCl salts.

TABLE 15
Histopathology Scores for Lungs of Mice 24 Hours After
LPS-Induced Acute Lung Injury

| | | Mean (SEM) | Mean (SEM) | Mean |
|---|---|---|---|---|
| | | Alveolar | Interstitial | (SEM) |
| | | Neutrophil | Neutrophil | Composite |
| | Peptide | Infiltration | Infiltration | Lung Injury |
| Treatment¹ | Dose | Score | Score | Score |
| Control (no LPS) | N/A | 0.00 (0.00) | 0.03 (0.03) | 0.13 (0.05) |
| LPS/Vehicle | N/A | 1.88 (0.05) | 1.80 (0.13) | 3.96 (0.16) |
| LPS/SEQ ID NO: 15 | 5 mg/kg | 1.80 (0.14) | 1.83 (0.17) | 3.83 (0.30) |
| LPS/SEQ ID NO: 15 | 15 mg/kg | 1.28 (0.15) | 1.15 (0.18) | 2.55 (0.28) |
| LPS/SEQ ID NO: 37 | 5 mg/kg | 1.50 (0.09) | 1.55 (0.14) | 3.08 (0.17) |
| LPS/SEQ ID NO: 37 | 15 mg/kg | 1.60 (0.13) | 1.23 (0.11) | 2.93 (0.22) |

¹Peptides were administered as HCl salts.

### Example 9

### Activation of Apelin Receptor (APJ) Downstream Signaling Via ERK1/2 in Cultured Cells

The effect of the peptides of the current invention on activation of Apelin Receptor (APJ)-mediated signaling can be assessed using an assay to monitor phospho-ERK/phospho-p44/42 MAPK (p-ERK1/2) levels in cultured cells overexpressing Apelin Receptor (APJ) such as CHO-K1, derived from Chinese hamster ovary. Peptides were initially prepared as 100× stocks in H₂O. Pyr-Apelin-13 (positive control) was initially prepared as 1 mM stock in H₂O and used as a potent positive control for APJ activation and downstream signaling. CHO-K1 AGTRL1 Gi cells stably overexpressing APJ were purchased from Eurofins-DiscoverX (Fremont, Calif.), seeded onto 6-well plates in standard culture medium (Ham's F12K+10% Fetal Bovine Serum+antibiotics) at 600,000 cells/well and allowed to adhere overnight at 37° C. in a humidified atmosphere of 5% CO₂/95% air. The next day, cells were spiked with vehicle (water), peptides (0.4 μM-50 μM final concentration), or Pyr-Apelin-13 (100 nM final concentration). Following a 5 min incubation the supernatant was removed and the cells were placed on ice and washed twice with cold HBSS. The HBSS was removed and the cells were immediately lysed in 300 μl/well of 1× lysis buffer (CST 10× Lysis Buffer; Cell Signaling Technology; Danvers, Mass.); lysis buffer was supplemented with 1 mM PMSF (Millipore-Sigma; Saint Louis, Mo.) and Halt Protease/Phosphatase Inhibitor Cocktail (Thermo-Fisher; Waltham, Mass.) prior to use. The samples were then placed on ice for 10 min, wells were scraped and lysates transferred to microfuge tubes. The samples were centrifuged at 12,500 rpm for 10 min at 4° C. and p-ERK1/2 expression in each sample was measured using Phospho-p44/42 MAPK (Thr202/Tyr204) Sandwich ELISA kit according to manufacturer's instructions (Cell Signaling Technology; Danvers, Mass.). Absorbance was measured using a Cytation 3 plate reader at 450 nM (BioTek; Winooski, Vt.). Data are presented as percent of the control effect for Pyr-Apelin-13 positive control. Each data point represents the average of duplicate assays. The results are shown in Table 16. Treatment with the peptides resulted in a dose-dependent increase in phosphorylation of ERK1/2, confirming that the peptides activate APJ-mediated downstream signaling via ERK1/2.

TABLE 16
Phosphorylation of ERK1/2 in Cultured Cells Overexpressing
Apelin Receptor (APJ)

| | Peptide | pERK1/2 |
|---|---|---|
| | Concentration | (% of Positive |
| Treatment¹ | (μM) | Control) |
| SEQ ID NO: 15 | 0.4 | 1.9 |
| SEQ ID NO: 15 | 2.0 | 15.4 |
| SEQ ID NO: 15 | 10 | 44.4 |
| SEQ ID NO: 15 | 50 | 69.7 |
| SEQ ID NO: 37 | 0.4 | 9.9 |
| SEQ ID NO: 37 | 2.0 | 27.3 |
| SEQ ID NO: 37 | 10 | 54.2 |
| SEQ ID NO: 37 | 50 | 88.4 |

¹Peptides were administered as acetate salts.

### Example 10

### Activation of Apelin Receptor (APJ) Downstream Signaling Vis MEK1 in Cultured Cells

The effect of the peptides on activation of Apelin Receptor (APJ) mediated signaling can be assessed using an assay to monitor phospho-MEK (p-MEK) levels in cultured cells overexpressing Apelin Receptor (APJ) such as CHO-K1, derived from Chinese hamster ovary. Peptides were initially prepared as 100× stocks in H2O. The endogenous APJ ligand Pyr-Apelin-13 (positive control) was initially prepared as 1 mM stock in H₂O and used as potent positive control for APJ activation and downstream signaling. CHO-K1 AGTRL1 Gi cells stably overexpressing APJ were purchased from Eurofins-DiscoverX (Fremont, Calif.), seeded onto 6-well plates in standard culture medium (Ham's F12K+10% Fetal Bovine Serum+antibiotics) at 600,000 cells/well, and allowed to adhere overnight at 37° C. in a humidified atmosphere of 5% CO₂/95% air. The next day, vehicle (H₂O), peptides (0.4 μM-50 μM final concentration), or Pyr-Apelin-13 (100 nM final concentration) were spiked into the cultures. Following a 5 min incubation, the supernatant was removed and the cells were placed on ice and washed twice with cold HBSS. The HBSS was removed and the cells were immediately lysed in 300 μl/well of 1× lysis buffer (CST 10× Lysis Buffer; Cell Signaling Technology; Danvers, Mass.); lysis buffer was supplemented with 1 mM PMSF (Millipore-Sigma; Saint Louis, Mo.) and Halt Protease/Phosphatase Inhibitor Cocktail (Thermo-Fisher; Waltham, Mass.) prior to use. The samples were then placed on ice for 10 min, the wells were scraped and lysates were centrifuged at 12,500 rpm for 10 min at 4° C., and p-MEK expression in each sample was measured using Phospho-MEK1 (Ser217/221) Sandwich ELISA kit according to manufacturer's instructions (Cell Signaling Technology; Danvers, Mass.). Absorbance was measured using a Cytation 3 plate reader at 450 nM (BioTek; Winooski, Vt.). Data are presented as percent of the control effect for Pyr-Apelin-13 positive control. Each data point represents the average of duplicate assays. The results are shown in Table 17. Treatment with the peptides resulted in a dose-dependent increase in phosphorylation of MEK1, confirming that they elicit APJ-mediated downstream signaling via MEK1.

TABLE 17
Phosphorylation of MEK1 in Cultured Cells Overexpressing
Apelin Receptor (APJ)

| | Peptide | pMEK1 |
|---|---|---|
| | Concentration | (% of Positive |
| Treatment¹ | (μM) | Control) |
| SEQ ID NO: 15 | 2.0 | 2.5 |
| SEQ ID NO: 15 | 10 | 14.3 |
| SEQ ID NO: 15 | 50 | 52.7 |
| SEQ ID NO: 37 | 2.0 | 8.7 |
| SEQ ID NO: 37 | 10 | 45.5 |
| SEQ ID NO: 37 | 50 | 79.4 |

¹Peptides were administered as acetate salts.

### Example 11

### Activation of Apelin Receptor (APJ) Downstream Signaling Via Ras in Cultured Cells

The effect of the peptides on activation of Apelin Receptor (APJ)-mediated signaling can be assessed using an assay to monitor GTP-bound/active Ras levels in cultured cells overexpressing Apelin Receptor (APJ) such as CHO-K1, derived from Chinese hamster ovary. Peptides were initially prepared as 100× stocks in H₂O. The endogenous APJ ligand Pyr-Apelin-13 (positive control) was initially prepared as 1 mM stock in H₂O and used as potent positive control for APJ activation and downstream signaling. CHO-K1 AGTRL1 Gi cells stably overexpressing APJ were purchased from Eurofins-DiscoverX (Fremont, Calif.). CHO-K1 AGTRL1 Gi cells were seeded onto 6-well plates in standard culture medium (Ham's F12K+10% Fetal Bovine Serum+antibiotics) at 600,000 cells/well and allowed to adhere overnight at 37° C. in a humidified atmosphere of 5% CO₂/95% air. The next day vehicle (H₂O), peptides (2 μM-50 μM final concentration), or Pyr-Apelin-13 (100 nM final concentration) were spiked into the cultures. Following a 1 min incubation, the cells were placed on ice, the supernatant was removed, and washed twice with cold HBSS. The HBSS was removed and the cells were immediately lysed in 150 μl/well of lysis buffer included with Active Ras Detection kit (Cell Signaling Technology; Danvers, Mass.); lysis buffer was supplemented with 1 mM PMSF (Millipore-Sigma; Saint Louis, Mo.) and Halt Protease/Phosphatase Inhibitor Cocktail (Thermo-Fisher; Waltham, Mass.) prior to use. The wells were immediately scraped and lysates transferred to microfuge tubes. The samples were then placed on ice for 10 min. Following the lysis, samples were centrifuged at 12,500 rpm for 10 min at 4° C. and GTP-bound Ras levels in each of the samples was determined following affinity capture of GTP-Ras, elution, and Western Blotting of isolated Ras using Active Ras Detection kit according to manufacturer's instructions. Visualization and quantification of Ras recovery (GTP-bound/active Ras) in samples were performed with Azure Biosystems C400 Imager and AzureSpot analysis software (Dublin, Calif.). Data are presented as percent of the control effect for Pyr-Apelin-13 positive control. Each data point represents the average of duplicate assays. The results are shown in Table 18. Treatment with the peptides resulted in an increase in activation of Ras, confirming that they elicit APJ-mediated downstream signaling via Ras.

TABLE 18
Activation of Ras in Cultured Cells Overexpressing Apelin Receptor (APJ)

| | Peptide | GTP-Ras |
|---|---|---|
| | Concentration | (% of Positive |
| Treatment | (μM) | Control) |
| SEQ ID NO: 15 | 50 | 64.9 |
| Acetate salt | | |
| SEQ ID NO: 15 | 50 | 90.8 |
| HCl salt | | |
| SEQ ID NO: 37 | 50 | 79.2 |
| Acetate salt | | |
| SEQ ID NO: 37 | 50 | 33.2 |
| HCl salt | | |

### Example 12

### Syrian Hamster Animal Model for SARS/COVID-19

The effect of the peptides on the pathological manifestations of COVID-19 can be assessed using an assay as described by Imai et al, PNAS, 117, 16587-95 (2020) or Chan et al., Clin. Infect. Dis., 71, 2428-46 (2020), each incorporated herein by reference in their entirety and particularly for the assays materials and methods. Such pathologies include lung weight or chemokine/cytokine levels.

All of the articles and methods disclosed and claimed herein can be made and executed without undue experimentation in light of the present disclosure. While the articles and methods of this disclosure have been described in terms of preferred embodiments, it will be apparent to those of skill in the art that variations may be applied to the articles and methods without departing from the spirit and scope of the disclosure. All such variations and equivalents apparent to those skilled in the art, whether now existing or later developed, are deemed to be within the spirit and scope of the disclosure as defined by the appended claims. All patents, patent applications, and publications mentioned in the specification are indicative of the levels of those of ordinary skill in the art to which the disclosure pertains. The disclosure illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure claimed. Thus, it should be understood that although the present disclosure has been specifically disclosed by preferred embodiments and optional features, modification and variation of the concepts herein disclosed may be resorted to by those skilled in the art, and that such modifications and variations are considered to be within the scope of this disclosure as defined by the appended claims.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law). All headings and sub-headings are used herein for convenience only and should not be construed as being limiting in any way. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure. The citation and incorporation of patent documents herein is done for convenience only and does not reflect any view of the validity, patentability, and/or enforceability of such patent documents.

This disclosure includes all modifications and equivalents of the subject matter recited in the aspects appended hereto as permitted by applicable law.

The present application includes a Sequence Listing. To the extent differences exist between information/description of sequences in the specification and information in the Sequence Listing, the specification is controlling.

What is claimed:

1. A method of treating a coronavirus infection in a subject, comprising administering to the subject a peptide comprising an amino acid sequence of Formula I:
(I)
(SEQ ID NO: 1)
X¹-RX²-X³-X⁴-X⁵-X⁶-Q-X⁷-L-X⁸-X⁹
wherein X¹ is absent or if present is an amino acid having a polar side chain or a non-polar side chain; X² is an amino acid having a polar side chain or a non-polar side chain; X³ is absent or if present is one to three amino acids, each amino acid independently having a polar side chain or a non-polar side chain; X⁴ is an amino acid having a polar side chain or a non-polar side chain; X⁵ is an amino acid having a non-polar side chain; X⁶ is an amino acid having a polar side chain or a non-polar side chain; X⁷ is an amino acid having a polar side chain; X⁸ is an amino acid having a polar side chain; and X⁹ is absent or if present is one to three amino acids, each amino acid independently having a polar side chain or a non-polar side chain;
or administering an analog of said peptide having a deletion, insertion or substitution of one, two, three, or four amino acids; or administering a C-terminal acid or amide, or N-acetyl derivative thereof; or administering a pegylated derivative thereof; or administering a pharmaceutically acceptable salt thereof.

2. A peptide, peptide analog, or derivative thereof, or a pharmaceutically acceptable salt thereof for use in the treatment of a subject having or suspected of having a coronavirus infection, the peptide comprising an amino acid sequence of Formula I:
(I)
(SEQ ID NO: 1)
X¹-RX²-X³-X⁴-X⁵-X⁶-Q-X⁷-L-X⁸-X⁹
wherein X¹ is absent or if present is an amino acid having a polar side chain or a non-polar side chain; X² is an amino acid having a polar side chain or a non-polar side chain; X³ is absent or if present is one to three amino acids, each amino acid independently having a polar side chain or a non-polar side chain; X⁴ is an amino acid having a polar side chain or a non-polar side chain; X⁵ is an amino acid having a non-polar side chain; X⁶ is an amino acid having a polar side chain or a non-polar side chain; X⁷ is an amino acid having a polar side chain; X⁸ is an amino acid having a polar side chain; and X⁹ is absent or if present is one to three amino acids, each amino acid independently having a polar side chain or a non-polar side chain; the analog of said peptide having a deletion, insertion or substitution of one, two, three, or four amino acids; the derivative comprising a C-terminal acid or amide, or a N-acetyl derivative, or a pegylated derivative.

3. A method of treating a subject in need of treatment for sepsis, septic shock, ischemic shock, or organ failure associated with a viral infection, the method comprising administering to the subject a peptide comprising an amino acid sequence of Formula I:
(I)
(SEQ ID NO: 1)
X¹-RX²-X³-X⁴-X⁵-X⁶-Q-X⁷-L-X⁸-X⁹
wherein X¹ is absent or if present is an amino acid having a polar side chain or a non-polar side chain; X² is an amino acid having a polar side chain or a non-polar side chain; X³ is absent or if present is one to three amino acids, each amino acid independently having a polar side chain or a non-polar side chain; X⁴ is an amino acid having a polar side chain or a non-polar side chain; X⁵ is an amino acid having a non-polar side chain; X⁶ is an amino acid having a polar side chain or a non-polar side chain; X⁷ is an amino acid having a polar side chain; X⁸ is an amino acid having a polar side chain; and X⁹ is absent or if present is one to three amino acids, each amino acid independently having a polar side chain or a non-polar side chain;
or administering an analog of said peptide having a deletion, insertion or substitution of one, two, three, or four amino acids; or administering a derivative comprising a C-terminal acid or amide, or a N-acetyl derivative thereof, or a pegylated derivative thereof; or administering a pharmaceutically acceptable salt thereof.

4. A peptide, peptide analog, or derivative thereof, or a pharmaceutically acceptable salt thereof for use in the treatment of sepsis, septic shock, ischemic shock, or organ failure associated with a viral infection, the peptide comprising an amino acid sequence of Formula I:
(I)
(SEQ ID NO: 1)
X¹-RX²-X³-X⁴-X⁵-X⁶-Q-X⁷-L-X⁸-X⁹
wherein X¹ is absent or if present is an amino acid having a polar side chain or a non-polar side chain; X² is an amino acid having a polar side chain or a non-polar side chain; X³ is absent or if present is one to three amino acids, each amino acid independently having a polar side chain or a non-polar side chain; X⁴ is an amino acid having a polar side chain or a non-polar side chain; X⁵ is an amino acid having a non-polar side chain; X⁶ is an amino acid having a polar side chain or a non-polar side chain; X⁷ is an amino acid having a polar side chain; X⁸ is an amino acid having a polar side chain; and X⁹ is absent or if present is one to three amino acids, each amino acid independently having a polar side chain or a non-polar side chain; the analog of said peptide having a deletion, insertion or substitution of one, two, three, or four amino acids; the derivative comprising a C-terminal acid or amide, or a N-acetyl derivative thereof, or a pegylated derivative thereof.

5. The method or the peptide, analog, derivative, or salt for use of any one of claims 1 to 4, wherein X¹ is M, K, or absent; X² is R or Aib; X³ is absent or is M, E, -MMG-, -LLG-, -II(dA)-, -Nle-Nle-G- or -IIG-; X⁴ is M, E, L, I or Nle; X⁵ is V, A or G; X⁶ is F, Y, A or E; X⁷ is C, S or E; X⁸ is C, S or E; and X⁹ is -GL, -G(dA), -G(dA)K, -(dA)L, G or absent.

6. The method or the peptide, analog, derivative, or salt for use of any one of claims 1 to 4, wherein, in the peptide or derivative, X¹ is (PEG12)-K, and/or wherein X⁹ is -G(dA)-K(PEG12).

7. The method or the peptide, analog, derivative, or salt for use of any one of claims 1 to 4, wherein X³ is absent or is -LLG-; X⁴ is L; X⁵ is V; or X⁸ is C or E.

8. The method or the peptide, analog, derivative, or salt for use of any one of claims 1 to 4, wherein X⁷ is S.

9. The method or the peptide, analog, derivative, or salt for use of any one of claims 1 to 4, wherein the peptide or peptide derivative comprises or consists of an amino acid sequence selected from SEQ ID NOs: 2-63.

10. The method or the peptide, analog, derivative, or salt for use of any one of claims 1 to 4, wherein the peptide or peptide derivative comprises or consists of an amino acid sequence selected from MRRMMGMVFQCLCGL (SEQ ID NO: 7); RRMMGMVFQCLCG(dA) (SEQ ID NO: 8); RRMMGMVYQCLCG(dA) (SEQ ID NO: 10); RRMMGMVAQCLCG(dA) (SEQ ID NO: 11); RRMMGMVFQELCG(dA) (SEQ ID NO: 13); RRMMGMVFQCLEG(dA) (SEQ ID NO: 14); RRMMGMVFQSLCG(dA) (SEQ ID NO: 15); RR(Nle)(Nle)G(Nle)VFQCLCG(dA) (SEQ ID NO: 18); (PEG12)KRRMMGMVFQCLCG(dA) (SEQ ID NO: 20); RRMMGMVFQCLCG(dA)K(PEG12) (SEQ ID NO: 21); RRMVYQCLCG(dA) (SEQ ID NO: 22; RRMMGMVAQCLEG(dA) (SEQ ID NO: 30); R(Aib)MMGMVFQSLCG(dA) (SEQ ID NO: 34); (PEG12)KRRMMGMVFQSLCG(dA) (SEQ ID NO: 36); (PEG12)KRRLLGLVFQSLCG(dA) (SEQ ID NO: 37); (PEG12)KRRIIGIVFQCLCG(dA) (SEQ ID NO: 42); RRIIGIVFQSLCG(dA) (SEQ ID NO: 43).

11. A method of treating a coronavirus infection in a subject in need thereof, comprising administering to the subject a peptide comprising an amino acid sequence of Formula III′:
(III’)
(SEQ ID NO: 78)
X¹⁸-R-X¹⁹-X²⁰-X²¹ V-X²²-Q-X²³ L-X²⁴-G-X²⁵
wherein X¹⁸ is absent or if present is M or K; X¹⁹ is R or Aib; X²⁰ is absent or if present is -M-M-G- or Nle-Nle-G-; X²¹ is M or Nle; X²² is F, A or Y; X²³ is S, E or C; X²⁴ is E or C; X²⁵ is L, dA or -dA-K; or administering a C-terminal acid or amide thereof, or a N-acetyl derivative thereof; or administering a pegylated derivative thereof; or administering a pharmaceutically acceptable salt thereof.

12. A peptide, derivative thereof, or a pharmaceutically acceptable salt thereof for use in the treatment of a subject having or suspected of having a coronavirus infection, the peptide comprising an amino acid sequence of Formula III′:
(III’)
(SEQ ID NO: 78)
X¹⁸-R-X¹⁹-X²⁰-X²¹ V-X²²-Q-X²³ L-X²⁴-G-X²⁵
wherein X¹⁸ is absent or if present is M or K; X¹⁹ is R or Aib; X²⁰ is absent or if present is -M-M-G- or Nle-Nle-G-; X²¹ is M or Nle; X²² is F, A or Y; X²³ is S, E or C; X²⁴ is E or C; X²⁵ is L, dA or -dA-K; the derivative comprising a C-terminal acid or amide, or N-acetyl derivative thereof; or a pegylated derivative thereof.

13. A method of treating a subject in need of treatment for sepsis, septic shock, ischemic shock, or organ failure associated with a viral infection, the method comprising administering to the subject a peptide comprising an amino acid sequence of Formula III′:
(III’)
(SEQ ID NO: 78)
X¹⁸-R-X¹⁹-X²⁰-X²¹ V-X²²-Q-X²³ L-X²⁴-G-X²⁵
wherein X¹⁸ is absent or if present is M or K; X¹⁹ is R or Aib; X²⁰ is absent or if present is -M-M-G- or Nle-Nle-G-; X²¹ is M or Nle; X²² is F, A or Y; X²³ is S, E or C; X²⁴ is E or C; X²⁵ is L, dA or -dA-K; or administering a derivative thereof, the derivative comprising a C-terminal acid or amide, or N-acetyl derivative thereof; or a pegylated derivative thereof; or
administering a pharmaceutically acceptable salt thereof.

14. A peptide, derivative thereof, or a pharmaceutically acceptable salt thereof for use in the treatment of sepsis, septic shock, ischemic shock, or organ failure associated with a viral infection, the peptide comprising an amino acid sequence of Formula III′:
(III′)
(SEQ ID NO: 78)
X¹⁸-R-X¹⁹ -X²⁰-X²¹ V-X²²-Q-X²³ L-X²⁴-G-X²⁵
wherein X¹⁸ is absent or if present is M or K; X¹⁹ is R or Aib; X²⁰ is absent or if present is -M-M-G- or Nle-Nle-G-; X²¹ is M or Nle; X²² is F, A or Y; X²³ is S, E or C; X²⁴ is E or C; X²⁵ is L, dA or -dA-K; the derivative comprising a C-terminal acid or amide, or N-acetyl derivative thereof; or a pegylated derivative thereof.

15. The method or the peptide, derivative, or salt for use of any one of claims 11-14, wherein X²⁵ is dA.

16. The method or the peptide, derivative, or salt for use of any one of claims 11-14, wherein X¹⁹ is R; X²⁰ is absent or is -M-M-G-; and X²¹ is M.

17. The method or the peptide, derivative, or salt for use of any one of claims 11-14, wherein X²² is F; and X²³ is C.

18. The method or the peptide, derivative, or salt for use of any one of claims 11-14, wherein the peptide or derivative comprises or consists of an amino acid sequence selected from MRRMMGMVFQCLCGL (SEQ ID NO: 7); RRMMGMVFQSLCG(dA) (SEQ ID NO: 15); and (PEG12)KRRMMGMVFQSLCG(dA) (SEQ ID NO: 36).

19. The method or the peptide, derivative, or salt for use of any one of claims 11-14, wherein the peptide or derivative comprises or consists of an amino acid sequence selected from (PEG12)RRMMGMVFQSLCG(dA) (SEQ ID NO: 71); and (K(PEG12))RRMMGMVFQSLCG(dA) (SEQ ID NO: 72).

20. A method of treating a coronavirus infection in a subject in need thereof, comprising administering to the subject a peptide comprising an amino acid sequence of Formula IV:
(IV)
(SEQ ID NO: 70)
X²⁶-RR-X²⁷-X²⁸ G-X²⁹-VFQ-X³⁰-LCG-(dA)
wherein X²⁶ is absent or if present is K; X²⁷ is L or I; X²⁸ is L or I; X²⁹ is L or I; and X³⁰ is S or C;
or administering a C-terminal acid or amide, or N-acetyl derivative thereof; or administering a pegylated derivative thereof; or administering a pharmaceutically acceptable salt thereof.

21. A peptide, derivative thereof, or a pharmaceutically acceptable salt thereof for use in the treatment of a subject having or suspected of having a coronavirus infection, the peptide comprising an amino acid sequence of Formula IV:
(IV)
(SEQ ID NO: 70)
X²⁶-RR-X²⁷-X²⁸ G-X²⁹-VFQ-X³⁰-LCG-(dA)
wherein X²⁶ is absent or if present is K; X²⁷ is L or I; X²⁸ is L or I; X²⁹ is L or I; and X³⁰ is S or C; the derivative comprising a C-terminal acid or amide, or a N-acetyl derivative thereof; or a pegylated derivative thereof.

22. A method of treating a subject in need of treatment for sepsis, septic shock, ischemic shock, or organ failure associated with a viral infection, the method comprising administering to the subject a peptide comprising an amino acid sequence of Formula IV:
(IV)
(SEQ ID NO: 70)
X²⁶-RR-X²⁷-X²⁸ G-X²⁹-VFQ-X³⁰-LCG-(dA)
wherein X²⁶ is absent or if present is K; X²⁷ is L or I; X²⁸ is L or I; X²⁹ is L or I; and X³⁰ is S or C; or administering a derivative thereof, the derivative comprising a C-terminal acid or amide, or N-acetyl derivatives thereof; or a pegylated derivative thereof; or administering a pharmaceutically acceptable salt thereof.

23. A peptide, or derivative thereof, or pharmaceutically acceptable salt thereof, for use in the treatment of sepsis, septic shock, ischemic shock, or organ failure associated with a viral infection, the peptide comprising an amino acid sequence of Formula IV:
(IV)
(SEQ ID NO: 70)
X²⁶-RR-X²⁷-X²⁸ G-X²⁹-VFQ-X³⁰-LCG-(dA)
wherein X²⁶ is absent or if present is K; X²⁷ is L or I; X²⁸ is L or I; X²⁹ is L or I; and X³⁰ is S or C; the derivative comprising a C-terminal acid or amide, or a N-acetyl derivative thereof; or a pegylated derivative thereof.

24. The method or the peptide, derivative, or salt for use of any one of claims 20 to 23, wherein X³⁰ is S.

25. The method or the peptide, derivative, or salt for use of any one of claims 20 to 23, wherein X²⁷ is L; X²⁸ is L; and/or X²⁹ is L.

26. The method or the peptide, derivative, or salt for use of any one of claims 20 to 23, wherein the peptide or the derivative comprises or consists of an amino acid sequence selected from (PEG12)KRRLLGLVFQSLCG(dA) (SEQ ID NO: 37); or RRIIGIVFQSLCG(dA) (SEQ ID NO: 43).

27. The method or the peptide, derivative, or salt for use of any one of claims 20 to 23, wherein the peptide or the derivative comprises or consists of an amino acid sequence selected from (K(PEG12))RRLLGLVFQSLCG(dA) (SEQ ID NO: 73); (PEG12)RRLLGLVFQSLCG(dA) (SEQ ID NO: 74); (PEG12)KRRIIGIVFQSLCG(dA) (SEQ ID NO: 75); (K(PEG12))RRIIGIVFQSLCG(dA) (SEQ ID NO: 76); and (PEG12}RRIIGIVFQSLCG(dA) (SEQ ID NO: 77).

28. The method or the peptide, analog, derivative, or salt for use of any one of claims 1-2, 5-12, 15-21, and 24-27, wherein the coronavirus infection is SARS or COVID-19 infection.

29. The method or the peptide, analog, derivative, or salt for use of any one of claims 1-2, 5-12, 15-21, and 24-27, wherein the coronavirus infection causes acute lung injury or acute respiratory distress syndrome.

30. The method or the peptide, analog, derivative, or salt for use of any one of claims 1-2, 5-12, 15-21, and 24-27, wherein the coronavirus infection potentiates bacteria-induced acute lung damage.

31. The method or the peptide, analog, derivative, or salt for use of any one of claims 1-2, 5-12, 15-21, and 24-27, wherein the peptide is administered together with an agent for treating coronavirus-related symptoms.

32. A method of modulating pro-inflammatory cytokine secretion comprising administering to the subject a peptide comprising an amino acid sequence of Formula III′:
(III′)
(SEQ ID NO: 78)
X¹⁸-R-X¹⁹ -X²⁰-X²¹ V-X²²-Q-X²³ L-X²⁴-G-X²⁵
wherein X¹⁸ is absent or if present is M or K; X¹⁹ is R or Aib; X²⁰ is absent or if present is -M-M-G- or Nle-Nle-G-; X²¹ is M or Nle; X²² is F, A or Y; X²³ is S, E or C; X²⁴ is E or C; X²⁵ is L, dA or -dA-K; or administering a C-terminal acid or amide, or a N-acetyl derivative thereof; or administering a pegylated derivative thereof; or administering a pharmaceutically acceptable salt thereof.

33. A peptide, or derivative thereof, or pharmaceutically acceptable salt thereof, for use in the treatment of a subject having or suspected of having pro-inflammatory cytokine secretion, the peptide comprising an amino acid sequence of Formula III′:
(III′)
(SEQ ID NO: 78)
X¹⁸-R-X¹⁹ -X²⁰-X²¹ V-X²²-Q-X²³ L-X²⁴-G-X²⁵
wherein X¹⁸ is absent or if present is M or K; X¹⁹ is R or Aib; X²⁰ is absent or if present is -M-M-G- or Nle-Nle-G-; X²¹ is M or Nle; X²² is F, A or Y; X²³ is S, E or C; X²⁴ is E or C; X²⁵ is L, dA or -dA-K; the derivative comprising a C-terminal acid or amide, or N-acetyl derivative thereof; or a pegylated derivative thereof.

34. The method or the peptide, derivative, or salt for use of claim 32 or 33, wherein X²⁵ is dAf.

35. The method or the peptide, derivative, or salt for use of claim 32 or 33, wherein X¹⁹ is R; X²⁰ is absent or is -M-M-G-; and X²¹ is M.

36. The method or the peptide, derivative, or salt for use of claim 32 or 33, wherein X²² is F; and X²³ is C.

37. The method or the peptide, derivative, or salt for use of claim 32 or 33, wherein the peptide or derivative comprises or consists of an amino acid sequence selected from MRRMMGMVFQCLCGL (SEQ ID NO: 7); RRMMGMVFQSLCG(dA) (SEQ ID NO: 15); and (PEG12)KRRMMGMVFQSLCG(dA) (SEQ ID NO: 36).

38. The method or the peptide, derivative, or salt for use of claim 32 or 33, wherein the peptide or derivative comprises or consists of an amino acid sequence selected from (PEG12)RRMMGMVFQSLCG(dA) (SEQ ID NO: 71); and (K(PEG12))RRMMGMVFQSLCG(dA) (SEQ ID NO: 72).

39. A method of modulating pro-inflammatory cytokine secretion comprising administering to the subject a peptide comprising either an amino acid sequence of Formula IV:
(IV)
(SEQ ID NO: 70)
X²⁶-RR-X²⁷-X²⁸ G-X²⁹-VFQ-X³⁰-LCG-(dA)
wherein X²⁶ is absent or if present is K; X²⁷ is L or I; X²⁸ is L or I; X²⁹ is L or I; and X³⁰ is S or C; or administering a C-terminal acid or amide, or a N-acetyl derivative thereof; or administering a pegylated derivative thereof; or administering a pharmaceutically acceptable salt thereof.

40. A peptide, or derivative thereof, or pharmaceutically acceptable salt thereof for use in the treatment of a subject having or suspected of having pro-inflammatory cytokine secretion, the peptide comprising an amino acid sequence of Formula IV:
(IV)
(SEQ ID NO: 70)
X²⁶-RR-X²⁷-X²⁸ G-X²⁹-VFQ-X³⁰-LCG-(dA)
wherein X²⁶ is absent or if present is K; X²⁷ is L or I; X²⁸ is L or I; X²⁹ is L or I; and X³⁰ is S or C; the derivative comprising a C-terminal acid or amide, or N-acetyl derivative thereof; or a pegylated derivative thereof.

41. The method or the peptide, derivative, or salt for use of claim 39 or 40, wherein X³⁰ is S.

42. The method or the peptide, derivative, or salt for use of claim 39 or 40, wherein the peptide or derivative comprises or consists of an amino acid sequence selected from (PEG12)KRRLLGLVFQSLCG(dA) (SEQ ID NO: 37); or RRIIGIVFQSLCG(dA) (SEQ ID NO: 43).

43. The method or the peptide, derivative, or salt for use of claim 39 or 40, wherein the peptide or derivative comprises or consists of an amino acid sequence selected from (K(PEG12))RRLLGLVFQSLCG(dA) (SEQ ID NO: 73); (PEG12)RRLLGLVFQSLCG(dA) (SEQ ID NO: 74); (PEG12)KRRIIGIVFQSLCG(dA) (SEQ ID NO: 75); (K(PEG12))RRIIGIVFQSLCG(dA) (SEQ ID NO: 76); and (PEG12}RRIIGIVFQSLCG(dA) (SEQ ID NO: 77).

44. The method or use of any one of claims 32-43, wherein the pro-inflammatory cytokine is selected from one or more of IL-1p, IL-2, IL-4, IL-5, IL-6, IL-9, IL-10, IL-12p70, IL-17a, IL17γ, IL-17A, IL-17C, IL-17E/IL-25, IL-17A/F, IL-23, IL-27p28/IL-30, IL-31, TNFα, IFNγ, IP-10, MCP-1, MIP-1α, MIP-2, MIP-3a and IL-8

45. The method or use of any one of claims 32-43, wherein the pro-inflammatory cytokine secretion is reduced.

46. A method of modulating activation of Ras, or phosphorylation of MEK1 or ERK1/2 comprising administering to the subject a peptide comprising either an amino acid sequence of Formula III′ or IV:
(III′)
(SEQ ID NO: 78)
X¹⁸-R-X¹⁹ -X²⁰-X²¹-V-X²²-Q-X²³-L-X²⁴-G-X²⁵
wherein X¹⁸ is absent or if present is M or K; X¹⁹ is R or Aib; X²⁰ is absent or if present is -M-M-G- or Nle-Nle-G-; X²¹ is M or Nle; X²² is F, A or Y; X²³ is S, E or C; X²⁴ is E or C; X²⁵ is L, dA or -dA-K;
(IV)
(SEQ ID NO: 70)
X²⁶-RR-X²⁷-X²⁸ G-X²⁹-VFQ-X³⁰-LCG-(dA)
wherein X²⁶ is absent or if present is K; X²⁷ is L or I; X²⁸ is L or I; X²⁹ is L or I; and X³⁰ is S or C;
or administering a derivative thereof comprising a C-terminal acid or amide, or a N-acetyl derivative thereof; or a pegylated derivative thereof; or administering a pharmaceutically acceptable salt thereof.

47. A peptide, or derivative thereof, or pharmaceutically acceptable salt thereof for use in modulating activation of Ras, or phosphorylation of MEK1 or ERK1/2, peptide comprising either an amino acid sequence of Formula III′ or IV:
(III′)
(SEQ ID NO: 78)
X¹⁸-R-X¹⁹ -X²⁰-X²¹ V-X²²-Q-X²³-L-X²⁴-G-X²⁵
wherein X¹⁸ is absent or if present is M or K; X¹⁹ is R or Aib; X²⁰ is absent or if present is -M-M-G- or Nle-Nle-G-; X²¹ is M or Nle; X²² is F, A or Y; X²³ is S, E or C; X²⁴ is E or C; X²⁵ is L, dA or -dA-K;
(IV)
(SEQ ID NO: 70)
X²⁶-RR-X²⁷-X²⁸ G-X²⁹-VFQ-X³⁰-LCG-(dA)
wherein X²⁶ is absent or if present is K; X²⁷ is L or I; X²⁸ is L or I; X²⁹ is L or I; and X³⁰ is S or C;
the derivative thereof comprising a C-terminal acid or amide, or a N-acetyl derivative thereof; or a pegylated derivative thereof.

48. A method of treating a subject having or suspected of having a disease or disorder selected from extravascular lung fluid accumulation, infectious disease or acute lung injury, the method comprising administering to the subject a peptide comprising an amino acid sequence of Formula I:
(I)
(SEQ ID NO: 1)
X¹-RX²-X³-X⁴-X⁵-X⁶-Q-X⁷-L-X⁸-X⁹
wherein X¹ is absent or if present is an amino acid having a polar side chain or a non-polar side chain; X² is an amino acid having a polar side chain or a non-polar side chain; X³ is absent or if present is one to three amino acids, each amino acid independently having a polar side chain or a non-polar side chain; X⁴ is an amino acid having a polar side chain or a non-polar side chain; X⁵ is an amino acid having a non-polar side chain; X⁶ is an amino acid having a polar side chain or a non-polar side chain; X⁷ is an amino acid having a polar side chain; X⁸ is an amino acid having a polar side chain; and X⁹ is absent or if present is one to three amino acids, each amino acid independently having a polar side chain or a non-polar side chain;
or administering an analog of said peptide having a deletion, insertion or substitution of one, two, three, or four amino acids; or administering a derivative that comprises a C-terminal acid or amide, or N-acetyl derivative thereof; or a pegylated derivative therefore; or administering a pharmaceutically acceptable salt thereof.

49. A peptide, or analog thereof, or derivative thereof, or pharmaceutically acceptable salt thereof for use in the treatment of a subject having or suspected of having a disease or disorder selected from extravascular lung fluid accumulation. infectious disease or acute lung injury, the peptide comprising an amino acid sequence of Formula I:
(I)
(SEQ ID NO: 1)
X¹-RX²-X³-X⁴-X⁵-X⁶-Q-X⁷-L-X⁸-X⁹
wherein X¹ is absent or if present is an amino acid having a polar side chain or a non-polar side chain; X² is an amino acid having a polar side chain or a non-polar side chain; X³ is absent or if present is one to three amino acids, each amino acid independently having a polar side chain or a non-polar side chain; X⁴ is an amino acid having a polar side chain or a non-polar side chain; X⁵ is an amino acid having a non-polar side chain; X⁶ is an amino acid having a polar side chain or a non-polar side chain; X⁷ is an amino acid having a polar side chain; X⁸ is an amino acid having a polar side chain; and X⁹ is absent or if present is one to three amino acids, each amino acid independently having a polar side chain or a non-polar side chain; or an analog of said peptide having a deletion, insertion or substitution of one, two, three, or four amino acids; wherein the derivative comprises a C-terminal acid or amide, or N-acetyl derivative thereof; or a pegylated derivative thereof.

50. The method or the peptide, analog, derivative, or salt for use of claim 48 or 49, wherein X¹ is M, K, or absent; X² is R or Aib; X³ is absent or is M, E, -MMG-, -LLG-, -II(dA)-, -Nle-Nle-G- or -IIG-; X⁴ is M, E, L, I or Nle; X⁵ is V, A or G; X⁶ is F, Y, A or E; X⁷ is C, S or E; X⁸ is C, S or E; and X⁹ is -GL, -G(dA), -G(dA)K, -(dA)L, G or absent.

51. The method or the peptide, analog, derivative, or salt for use of claim 48 or 49, wherein, in the peptide, analog, or derivative, X¹ is (PEG12)-K, and/or wherein X⁹ is -G(dA)-K(PEG12).

52. The method or the peptide, analog, derivative, or salt for use of claim 48 or 49, wherein X³ is absent or is -LLG-; X⁴ is L; X⁵ is V; or X⁸ is C or E.

53. The method or the peptide, analog, derivative, or salt for use of claim 48 or 49, wherein the peptide or derivative comprises or consists of an amino acid sequence selected from SEQ ID NOs: 2-63.

54. The method or the peptide, analog, derivative, or salt for use of claim 48 or 49, wherein the peptide or derivative comprises or consists of MRRMMGMVFQCLCGL (SEQ ID NO: 7); RRMMGMVFQCLCG(dA) (SEQ ID NO: 8); RRMMGMVYQCLCG(dA) (SEQ ID NO: 10); RRMMGMVAQCLCG(dA) (SEQ ID NO: 11); RRMMGMVFQELCG(dA) (SEQ ID NO: 13); RRMMGMVFQCLEG(dA) (SEQ ID NO: 14); RRMMGMVFQSLCG(dA) (SEQ ID NO: 15); RR(Nle)(Nle)G(Nle)VFQCLCG(dA) (SEQ ID NO: 18); (PEG12)KRRMMGMVFQCLCG(dA) (SEQ ID NO: 20); RRMMGMVFQCLCG(dA)K(PEG12) (SEQ ID NO: 21); RRMVYQCLCG(dA) (SEQ ID NO: 22); RRMMGMVAQCLEG(dA) (SEQ ID NO: 30); R(Aib)MMGMVFQSLCG(dA) (SEQ ID NO: 34); (PEG12)KRRMMGMVFQSLCG(dA) (SEQ ID NO: 36); (PEG12)KRRLLGLVFQSLCG(dA) (SEQ ID NO: 37); (PEG12)KRRIIGIVFQCLCG(dA) (SEQ ID NO: 42); or RRIIGIVFQSLCG(dA) (SEQ ID NO: 43).

55. The method or use of any one of claims 1-54, wherein the pharmaceutically acceptable salt is an acetate or hydrochoride salt.

56. The method or the peptide, analog, derivative, or salt for use of any one of claim 10, 19, 26, 37 or 42, wherein the peptide or derivative or salt comprises or consists of (PEG12)KRRLLGLVFQSLCG(dA) (SEQ ID NO: 37) acetate, RRMMGMVFQSLCG(dA) (SEQ ID NO: 15) acetate, (PEG12)KRRLLGLVFQSLCG(dA) (SEQ ID NO: 37) hydrochloride, or RRMMGMVFQSLCG(dA) (SEQ ID NO: 15) hydrochloride.


SEQUENCE LISTING (79 sequences)

SEQ ID NO: 1 - protein, 16 aa, Artificial Sequence
  Synthetic peptide
  MISC_FEATURE (1)..(1): Xaa is absent or if present is an amino acid having a polar side chain or a non-polar side chain
  MISC_FEATURE (3)..(3): Xaa is an amino acid having a polar side chain or a non-polar side chain
  MISC_FEATURE (4)..(4): Xaa is absent or if present is an amino acids having a polar side chain or a non-polar side chain
  MISC_FEATURE (5)..(5): Xaa is absent or if present is an amino acids having a polar side chain or a non-polar side chain
  MISC_FEATURE (6)..(6): Xaa is absent or if present is an amino acids having a polar side chain or a non-polar side chain
  MISC_FEATURE (7)..(7): Xaa is an amino acid having a polar side chain or a non-polar side chain
  MISC_FEATURE (8)..(8): Xaa is an amino acid having a non-polar side chain
  MISC_FEATURE (9)..(9): Xaa  is an amino acid having a polar side chain or a non-polar side chain
  MISC_FEATURE (11)..(11): Xaa is an amino acid having a polar side chain
  MISC_FEATURE (13)..(13): Xaa is an amino acid having a polar side chain
  MISC_FEATURE (14)..(14): Xaa is absent or if present is an amino acid having a polar side chain or a non-polar side chain
  MISC_FEATURE (15)..(15): Xaa is absent or if present is an amino acid having a polar side chain or a non-polar side chain
  MISC_FEATURE (16)..(16): Xaa is absent or if present is an amino acid having a polar side chain or a non-polar side chain
XRXXXXXXXQXLXXXX

SEQ ID NO: 2 - protein, 15 aa, Artificial Sequence
  Synthetic peptide
MRRIIGIVFQCLCGL

SEQ ID NO: 3 - protein, 14 aa, Artificial Sequence
  Synthetic peptide
RRIIGIVFQCLCGL

SEQ ID NO: 4 - protein, 13 aa, Artificial Sequence
  Synthetic peptide
RRIIGIVFQCLCG

SEQ ID NO: 5 - protein, 12 aa, Artificial Sequence
  Synthetic peptide
RRIIGIVFQCLC

SEQ ID NO: 6 - protein, 14 aa, Artificial Sequence
  Synthetic peptide
  MISC_FEATURE (13)..(13): D-Ala
RRIIGIVFQCLCAL

SEQ ID NO: 7 - protein, 15 aa, Artificial Sequence
  Synthetic peptide
MRRMMGMVFQCLCGL

SEQ ID NO: 8 - protein, 14 aa, Artificial Sequence
  Synthetic peptide
  MISC_FEATURE (14)..(14): D-Ala
RRMMGMVFQCLCGA

SEQ ID NO: 9 - protein, 14 aa, Artificial Sequence
  Synthetic peptide
  MISC_FEATURE (5)..(5): D-Ala
  MISC_FEATURE (13)..(13): D-Ala
RRIIAIVFQCLCAL

SEQ ID NO: 10 - protein, 14 aa, Artificial Sequence
  Synthetic peptide
  MISC_FEATURE (14)..(14): D-Ala
RRMMGMVYQCLCGA

SEQ ID NO: 11 - protein, 14 aa, Artificial Sequence
  Synthetic peptide
RRMMGMVAQCLCGA

SEQ ID NO: 12 - protein, 14 aa, Artificial Sequence
  Synthetic peptide
  MISC_FEATURE (14)..(14): D-Ala
RRMMGMVEQCLCGA

SEQ ID NO: 13 - protein, 14 aa, Artificial Sequence
  Synthetic peptide
  MISC_FEATURE (14)..(14): D-Ala
RRMMGMVFQELCGA

SEQ ID NO: 14 - protein, 14 aa, Artificial Sequence
  Synthetic peptide
  MISC_FEATURE (14)..(14): D-Ala
RRMMGMVFQCLEGA

SEQ ID NO: 15 - protein, 14 aa, Artificial Sequence
  Synthetic peptide
  MISC_FEATURE (14)..(14): D-Ala
RRMMGMVFQSLCGA

SEQ ID NO: 16 - protein, 14 aa, Artificial Sequence
  Synthetic peptide
  MISC_FEATURE (14)..(14): D-Ala
RRMMGMVFQCLSGA

SEQ ID NO: 17 - protein, 14 aa, Artificial Sequence
  Synthetic peptide
  MISC_FEATURE (14)..(14): D-Ala
RRMMGMVFQSLSGA

SEQ ID NO: 18 - protein, 14 aa, Artificial Sequence
  Synthetic peptide
  MISC_FEATURE (3)..(3): Xaa is Norleucine
  MISC_FEATURE (4)..(4): Xaa is Norleucine
  MISC_FEATURE (6)..(6): Xaa is Norleucine
  MISC_FEATURE (14)..(14): D-Ala
RRXXGXVFQCLCGA

SEQ ID NO: 19 - protein, 11 aa, Artificial Sequence
  Synthetic peptide
  MISC_FEATURE (11)..(11): D-Ala
RRMVFQCLCGA

SEQ ID NO: 20 - protein, 15 aa, Artificial Sequence
  Synthetic peptide
  MISC_FEATURE (1)..(1): PEG12
  MISC_FEATURE (15)..(15): d-Ala
KRRMMGMVFQCLCGA

SEQ ID NO: 21 - protein, 15 aa, Artificial Sequence
  Synthetic peptide
  MISC_FEATURE (14)..(14): D-Ala
  MISC_FEATURE (15)..(15): PEG-12
RRMMGMVFQCLCGAK

SEQ ID NO: 22 - protein, 11 aa, Artificial Sequence
  Synthetic peptide
  MISC_FEATURE (11)..(11): D-Ala
RRMVYQCLCGA

SEQ ID NO: 23 - protein, 11 aa, Artificial Sequence
  Synthetic peptide
  MISC_FEATURE (11)..(11): D-Ala
RRMVFQCLEGA

SEQ ID NO: 24 - protein, 11 aa, Artificial Sequence
  Synthetic peptide
  MISC_FEATURE (11)..(11): D-Ala
RRMVYQCLEGA

SEQ ID NO: 25 - protein, 12 aa, Artificial Sequence
  Synthetic peptide
  MISC_FEATURE (12)..(12): D-Ala
RREMVYQCLCGA

SEQ ID NO: 26 - protein, 12 aa, Artificial Sequence
  Synthetic peptide
  MISC_FEATURE (12)..(12): D-Ala
RREMVYQCLEGA

SEQ ID NO: 27 - protein, 11 aa, Artificial Sequence
  Synthetic peptide
  MISC_FEATURE (11)..(11): D-Ala
RRMAYQCLEGA

SEQ ID NO: 28 - protein, 11 aa, Artificial Sequence
  Synthetic peptide
  MISC_FEATURE (11)..(11): D-Ala
RRMGYQCLEGA

SEQ ID NO: 29 - protein, 14 aa, Artificial Sequence
  Synthetic peptide
  MISC_FEATURE (14)..(14): D-Ala
RRMMGMVYQCLEGA

SEQ ID NO: 30 - protein, 14 aa, Artificial Sequence
  Synthetic peptide
  MISC_FEATURE (14)..(14): RRMMGMVAQCLEG(A
RRMMGMVAQCLEGA

SEQ ID NO: 31 - protein, 12 aa, Artificial Sequence
  Synthetic peptide
  MISC_FEATURE (13)..(13): D-Ala
RRMEVYQCLCGA

SEQ ID NO: 32 - protein, 12 aa, Artificial Sequence
  Synthetic peptide
  MISC_FEATURE (12)..(12): D-Ala
RRMEVYQCLEGA

SEQ ID NO: 33 - protein, 14 aa, Artificial Sequence
  Synthetic Polypeptide
  MISC_FEATURE (14)..(14): D-Ala
RRLLGLVFQSLCGA

SEQ ID NO: 34 - protein, 14 aa, Artificial Sequence
  Synthetic Polypeptide
  MISC_FEATURE (2)..(2): Xaa is Aib (alpha-aminoisobutyric acid)
  MISC_FEATURE (14)..(14): D-Ala
RXMMGMVFQSLCGA

SEQ ID NO: 35 - protein, 14 aa, Artificial Sequence
  Synthetic Polypeptide
  MISC_FEATURE (2)..(2): Xaa is Aib (alpha-aminoisobutyric acid)
  MISC_FEATURE (14)..(14): D-Ala
RXLLGLVFQSLCGA

SEQ ID NO: 36 - protein, 15 aa, Artificial Sequence
  Synthetic Polypeptide
  MISC_FEATURE (1)..(1): PEG12
  MISC_FEATURE (15)..(15): D-Ala
KRRMMGMVFQSLCGA

SEQ ID NO: 37 - protein, 15 aa, Artificial Sequence
  Synthetic Polypeptide
  MISC_FEATURE (1)..(1): PEG12
  MISC_FEATURE (15)..(15): D-Ala
KRRLLGLVFQSLCGA

SEQ ID NO: 38 - protein, 14 aa, Artificial Sequence
  Synthetic Polypeptide
  MISC_FEATURE (14)..(14): D-Ala
RRMMGMVEQSLCGA

SEQ ID NO: 39 - protein, 14 aa, Artificial Sequence
  Synthetic Polypeptide
  MISC_FEATURE (14)..(14): D-Ala
RRMMGMVFQSLEGA

SEQ ID NO: 40 - protein, 14 aa, Artificial Sequence
  Synthetic Polypeptide
  MISC_FEATURE (14)..(14): D-Ala
RRLLGLVEQSLCGA

SEQ ID NO: 41 - protein, 14 aa, Artificial Sequence
  Synthetic Polypeptide
  MISC_FEATURE (14)..(14): D-Ala
RRLLGLVFQSLEGA

SEQ ID NO: 42 - protein, 15 aa, Artificial Sequence
  Synthetic Polypeptide
  MISC_FEATURE (1)..(1): PEG12
  MISC_FEATURE (15)..(15): D-Ala
KRRIIGIVFQCLCGA

SEQ ID NO: 43 - protein, 14 aa, Artificial Sequence
  Synthetic Polypeptide
  MISC_FEATURE (14)..(14): D-Ala
RRIIGIVFQSLCGA

SEQ ID NO: 44 - protein, 5 aa, Artificial Sequence
  Synthetic Polypeptide
MMGMV

SEQ ID NO: 45 - protein, 6 aa, Artificial Sequence
  Synthetic Polypeptide
MMGMVF

SEQ ID NO: 46 - protein, 7 aa, Artificial Sequence
  Synthetic Polypeptide
MMGMVFQ

SEQ ID NO: 47 - protein, 8 aa, Artificial Sequence
  Synthetic Polypeptide
MMGMVFQS

SEQ ID NO: 48 - protein, 9 aa, Artificial Sequence
  Synthetic Polypeptide
MMGMVFQSL

SEQ ID NO: 49 - protein, 12 aa, Artificial Sequence
  Synthetic Polypeptide
  MISC_FEATURE (12)..(12): D-Ala
MMGMVFQSLCGA

SEQ ID NO: 50 - protein, 7 aa, Artificial Sequence
  Synthetic Polypeptide
RMMGMVF

SEQ ID NO: 51 - protein, 8 aa, Artificial Sequence
  Synthetic Polypeptide
RMMGMVFQ

SEQ ID NO: 52 - protein, 9 aa, Artificial Sequence
  Synthetic Polypeptide
RMMGMVFQS

SEQ ID NO: 53 - protein, 10 aa, Artificial Sequence
  Synthetic Polypeptide
RMMGMVFQSL

SEQ ID NO: 54 - protein, 13 aa, Artificial Sequence
  Synthetic Polypeptide
  MISC_FEATURE (13)..(13): D-Ala
RMMGMVFQSLCGA

SEQ ID NO: 55 - protein, 6 aa, Artificial Sequence
  Synthetic Polypeptide
RRMMGM

SEQ ID NO: 56 - protein, 7 aa, Artificial Sequence
  Synthetic Polypeptide
RRMMGMV

SEQ ID NO: 57 - protein, 8 aa, Artificial Sequence
  Synthetic Polypeptide
RRMMGMVF

SEQ ID NO: 58 - protein, 9 aa, Artificial Sequence
  Synthetic Polypeptide
RRMMGMVFQ

SEQ ID NO: 59 - protein, 10 aa, Artificial Sequence
  Synthetic Polypeptide
RRMMGMVFQS

SEQ ID NO: 60 - protein, 11 aa, Artificial Sequence
  Synthetic Polypeptide
RRMMGMVFQSL

SEQ ID NO: 61 - protein, 14 aa, Artificial Sequence
  Synthetic Polypeptide
  MISC_FEATURE (1)..(1): Acetyl
  MISC_FEATURE (14)..(14): D-Ala
RRMMGMVFQSLCGA

SEQ ID NO: 62 - protein, 14 aa, Artificial Sequence
  Synthetic Polypeptide
  MISC_FEATURE (14)..(14): D-Ala
  MISC_FEATURE (14)..(14): Amide
RRMMGMVFQSLCGA

SEQ ID NO: 63 - protein, 14 aa, Artificial Sequence
  Synthetic Polypeptide
  MISC_FEATURE (1)..(1): Acetyl
  MISC_FEATURE (63)..(63): D-Ala
  MISC_FEATURE (63)..(63): Amide
RRMMGMVFQSLCGA

SEQ ID NO: 64 - protein, 14 aa, Artificial Sequence
  Synthetic Polypeptide
  MISC_FEATURE (1)..(1): Xaa is absent or if present is Arg
  MISC_FEATURE (2)..(2): Xaa is absent or if present is Arg
  MISC_FEATURE (7)..(7): Xaa is absent or if present is Val
  MISC_FEATURE (8)..(8): Xaa is absent or if present is Phe. Xaa8 is absent if Xaa7 is absent
  MISC_FEATURE (9)..(9): Xaa is absent or if present is Gln. Xaa9 is absent if Xaa8 is absent
  MISC_FEATURE (10)..(10): Xaa is absent or if present is Ser. Xaa10 is absent if Xaa9 is absent
  MISC_FEATURE (11)..(11): Xaa is absent or if present is Leu. Xaa11 is absent if Xaa10 is absent
  MISC_FEATURE (12)..(12): Xaa is absent or if present is Cys. Xaa12 is absent if Xaa11 is absent
  MISC_FEATURE (13)..(13): Xaa is absent or if present is Gly. Xaa13 is absent if Xaa11 is absent. Xaa13 is present if Xaa12 is present
  MISC_FEATURE (14)..(14): Xaa is absent or if present is d-Ala. Xaa14 is absent if Xaa11 is absent. Xaa 14 is present if Xaa13 is present
XXMMGMXXXXXXXX

SEQ ID NO: 65 - protein, 11 aa, Artificial Sequence
  Synthetic Polypeptide
YARAAARQARA

SEQ ID NO: 66 - protein, 7 aa, Artificial Sequence
  Synthetic Polypeptide
YGRKKRR

SEQ ID NO: 67 - protein, 11 aa, Artificial Sequence
  Synthetic Polypeptide
YGRKKRRQRRR

SEQ ID NO: 68 - protein, 5 aa, Artificial Sequence
  Synthetic Polypeptide
RRQRR

SEQ ID NO: 69 - protein, 15 aa, Artificial Sequence
  Synthetic Polypeptide
  MISC_FEATURE (1)..(1): Xaa is absent or M or K
  MISC_FEATURE (2)..(2): Xaa is R or Aib
  MISC_FEATURE (3)..(3): Xaa is absent or is -M or Nle
  MISC_FEATURE (4)..(4): Xaa is absent if Xaa3 is absent.  Xaa is M if Xaa3 is M.  Xaa is Nle if Xaa3 is Nle
  MISC_FEATURE (5)..(5): Xaa is absent if Xaa3 is absent.  Xaa is G if Xaa3 is M or Nle.
  MISC_FEATURE (6)..(6): Xaa is M or Nle
  MISC_FEATURE (8)..(8): Xaa is F, A or Y
  MISC_FEATURE (10)..(10): Xaa is S, E or C
  MISC_FEATURE (12)..(12): Xaa is E or C
  MISC_FEATURE (14)..(14): Xaa is L or dA
  MISC_FEATURE (15)..(15): Xaa is absent or K, Xaa is absent if Xaa 14 is L.
XXXXXXVXQXIXGXX

SEQ ID NO: 70 - protein, 15 aa, Artificial Sequence
  Synthetic Polypeptide
  MISC_FEATURE (1)..(1): Xaa is absent or K
  MISC_FEATURE (4)..(4): Xaa is L or I
  MISC_FEATURE (5)..(5): Xaa is L or I
  MISC_FEATURE (7)..(7): Xaa is L or I
  MISC_FEATURE (11)..(11): Xaa is S or C
  MISC_FEATURE (15)..(15): d-Ala
XRRXXGXVFQXLCGA

SEQ ID NO: 71 - protein, 14 aa, Artificial Sequence
  Synthetic Polypeptide
  MISC_FEATURE (1)..(1): PEG12
  MISC_FEATURE (14)..(14): d-Ala
RRMMGMVFQSLCGA

SEQ ID NO: 72 - protein, 15 aa, Artificial Sequence
  Synthetic Polypeptide
  MISC_FEATURE (1)..(1): PEG12
  MISC_FEATURE (15)..(15): d-Ala
KRRMMGMVFQSLCGA

SEQ ID NO: 73 - protein, 15 aa, Artificial Sequence
  Synthetic Polypeptide
  MISC_FEATURE (1)..(1): PEG12
  MISC_FEATURE (15)..(15): d-Ala
KRRLLGLVFQSLCGA

SEQ ID NO: 74 - protein, 14 aa, Artificial Sequence
  Synthetic Polypeptide
  MISC_FEATURE (1)..(1): PEG12
  MISC_FEATURE (14)..(14): d-Ala
RRLLGLVFQSLCGA

SEQ ID NO: 75 - protein, 15 aa, Artificial Sequence
  Synthetic Polypeptide
  MISC_FEATURE (1)..(1): PEG12
  MISC_FEATURE (15)..(15): d-Ala
KRRIIGIVFQSLCGA

SEQ ID NO: 76 - protein, 15 aa, Artificial Sequence
  Synthetic Polypeptide
  MISC_FEATURE (1)..(1): PEG12
  MISC_FEATURE (15)..(15): d-Ala
KRRIIGIVFQSLCGA

SEQ ID NO: 77 - protein, 14 aa, Artificial Sequence
  Synthetic Polypeptide
  MISC_FEATURE (1)..(1): PEG12
  MISC_FEATURE (14)..(14): d-Ala
RRIIGIVFQSLCGA

SEQ ID NO: 78 - protein, 16 aa, Artificial Sequence
  Synthetic Polypeptide
  MISC_FEATURE (1)..(1): Xaa is absent or M or K
  MISC_FEATURE (3)..(3): Xaa is R or Aib
  MISC_FEATURE (4)..(4): Xaa is absent or M or Nle
  MISC_FEATURE (5)..(5): Xaa is absent if Xaa4 is absent; Xaa is M if Xaa4 is M.  Xaa is Nle if Xaa4 is Nle
  MISC_FEATURE (6)..(6): Xaa is absent or G; Xaa is absent if Xaa4 is absent; Xaa is G if Xaa4 is M or Nle.
  MISC_FEATURE (7)..(7): Xaa is M or Nle
  MISC_FEATURE (9)..(9): Xaa is F, A, or Y
  MISC_FEATURE (11)..(11): Xaa is S, E, or C
  MISC_FEATURE (13)..(13): Xaa is E or C
  MISC_FEATURE (15)..(15): Xaa is L or dA
  misc_feature (16)..(16): Xaa is absent or K; Xaa is absent if Xaa15 is L
XRXXXXXVXQXLXGXX

SEQ ID NO: 79 - protein, 14 aa, Artificial Sequence
  Synthetic Polypeptide
  MISC_FEATURE (1)..(1): Xaa is absent or R
  MISC_FEATURE (2)..(2): Xaa is absent, R, or Aib. Xaa is absent if Xaa1 is absent
  MISC_FEATURE (7)..(7): Xaa is absent or V
  MISC_FEATURE (8)..(8): Xaa is absent or F; Xaa is absent if Xaa7 is absent
  MISC_FEATURE (9)..(9): Xaa is absent or Q; Xaa is absent if Xaa8 is absent
  MISC_FEATURE (10)..(10): Xaa is absent or S; Xaa is absent if Xaa9 is absent
  MISC_FEATURE (11)..(11): Xaa is absent or L; Xaa is absent if Xaa10 is absent
  MISC_FEATURE (12)..(12): Xaa is absent or C; Xaa is absent if Xaa11 is absent
  MISC_FEATURE (13)..(13): Xaa is absent or G; Xaa is absent if Xaa11 is absent; Xaa is present if Xaa12 is present
  MISC_FEATURE (14)..(14): Xaa is absent or dA; Xaa is absent if Xaa11 is absent; Xaa is present if Xaa13 is present
XXMMGMXXXXXXXX